SEQUENCE LISTING (131 sequences)

SEQ ID NO: 1 - protein, 5 aa, synthetic construct
DYYMN

SEQ ID NO: 2 - protein, 17 aa, synthetic construct
DINPNNGGTSYNQKFKG

SEQ ID NO: 3 - protein, 14 aa, synthetic construct
GGAELTGTYWYFDV

SEQ ID NO: 4 - protein, 11 aa, synthetic construct
RASENIYSNLA

SEQ ID NO: 5 - protein, 7 aa, synthetic construct
GATNLAD

SEQ ID NO: 6 - protein, 9 aa, synthetic construct
QHFWDSPFT

SEQ ID NO: 7 - protein, 123 aa, synthetic construct
EVQLQQSGPELVKPGASVKISCKASGYTFTDYYMNWVKQSHGKSLEWIGDINPNNGGTSYNQKFKGKATLTVDKSSSTAYMELRSLTSEDSAVYYCARGGAELTGTYWYFDVWGTGTTVTVSS

SEQ ID NO: 8 - protein, 107 aa, synthetic construct
DIQMTQSPASLSISVGETVTITCRASENIYSNLAWYQQKQGKSPQVLVYGATNLADGVPSRFSGSGSGTQYSLKIKSLQSEDFGSYYCQHFWDSPFTFGSGTKLEIK

SEQ ID NO: 9 - protein, 5 aa, synthetic construct
GYSVN

SEQ ID NO: 10 - protein, 17 aa, synthetic construct
RINPYNGDIFNNQKFKG

SEQ ID NO: 11 - protein, 11 aa, synthetic construct
GTTVGGAWFAY

SEQ ID NO: 12 - protein, 15 aa, synthetic construct
RASESVDSYGYSFLH

SEQ ID NO: 13 - protein, 7 aa, synthetic construct
LASNLES

SEQ ID NO: 14 - protein, 10 aa, synthetic construct
QQSNEDLMYT

SEQ ID NO: 15 - protein, 120 aa, synthetic construct
DVQLQQSGPELVKPGNSVKISCKAAGYSFTGYSVNWVKERHGKSLEWIGRINPYNGDIFNNQKFKGKATLTVDKSSSTAHMELRSLTSEDSAVYYCARGTTVGGAWFAYWGQGTLVTVSA

SEQ ID NO: 16 - protein, 112 aa, synthetic construct
VIVLTQSPASLAVSLGQRAAISCRASESVDSYGYSFLHWYQQKPGQPPKLLIYLASNLESGIPARFSGSGSGTDFTLTINPVEADDVATYYCQQSNEDLMYTFGGGTKLEIK

SEQ ID NO: 17 - protein, 17 aa, synthetic construct
RIYPGVYRTHYNEKFKD

SEQ ID NO: 18 - protein, 15 aa, synthetic construct
SGYYGGTYEEDAMDY

SEQ ID NO: 19 - protein, 7 aa, synthetic construct
AATSLAD

SEQ ID NO: 20 - protein, 9 aa, synthetic construct
QNFWDTPYT

SEQ ID NO: 21 - protein, 124 aa, synthetic construct
QVQLKQSGAELVRPGASVKLSCRASGYTFTDYYMNWVKQRPGQGLEWIARIYPGVYRTHYNEKFKDKATLTAEKSSSTAYMELSSLTSEDSAVYFCARSGYYGGTYEEDAMDYWGQGTSVTVSS

SEQ ID NO: 22 - protein, 107 aa, synthetic construct
DIQMTQSPASLSVSVGETVTITCRASENIYSNLAWYQQKQGKSPQLLVYAATSLADGVPSRFRGSGSGTQYSLKISSLQSEDFGNYYCQNFWDTPYTFGGGTKLEIK

SEQ ID NO: 23 - protein, 5 aa, synthetic construct
DSYMN

SEQ ID NO: 24 - protein, 17 aa, synthetic construct
YINPDNGVTRYNQKFKG

SEQ ID NO: 25 - protein, 13 aa, synthetic construct
EGTITTDLSWFAY

SEQ ID NO: 26 - protein, 7 aa, synthetic construct
ASTNLAD

SEQ ID NO: 27 - protein, 9 aa, synthetic construct
QHFWDTPYT

SEQ ID NO: 28 - protein, 122 aa, synthetic construct
EVQLQQSGPELVIPGASVKISCKASGYTFTDSYMNWVKQSHGKSLEWIAYINPDNGVTRYNQKFKGKATLTVHKSSSTAYMELRSLTSEDSAVYYCAREGTITTDLSWFAYWGQGTLVTVSA

SEQ ID NO: 29 - protein, 107 aa, synthetic construct
DIQMTQSPASLSVSVGETVTITCRASENIYSNLAWYQQKQGKSPQLLVYASTNLADGAPATFSGSGSGTQYSLKINSLQSVDFGSYYCQHFWDTPYTFGGGTKLEIK

SEQ ID NO: 30 - protein, 5 aa, synthetic construct
GYFMN

SEQ ID NO: 31 - protein, 17 aa, synthetic construct
RINPYNGDIFYNQKFKG

SEQ ID NO: 32 - protein, 11 aa, synthetic construct
GITVAAGSFDV

SEQ ID NO: 33 - protein, 15 aa, synthetic construct
RASESVDNYGNTFMH

SEQ ID NO: 34 - protein, 7 aa, synthetic construct
RASNLES

SEQ ID NO: 35 - protein, 9 aa, synthetic construct
QQSSDHPLT

SEQ ID NO: 36 - protein, 120 aa, synthetic construct
EVHLQQSGPELVKPGASVKISCKASGYSFIGYFMNWMKQSHGKSLEWIGRINPYNGDIFYNQKFKGKATLTVDKSSTTAHMDLLSLTSEDFAVYYCARGITVAAGSFDVWGTGTTVTVSS

SEQ ID NO: 37 - protein, 111 aa, synthetic construct
DIVLTQSPASLAVSLGQRATISCRASESVDNYGNTFMHWYQQKPGQPPKLLIYRASNLESGIPARFSGSGSKTDFTLTINPVEADDVATYYCQQSSDHPLTFGAGTKLELS

SEQ ID NO: 38 - protein, 5 aa, synthetic construct
SYWMN

SEQ ID NO: 39 - protein, 17 aa, synthetic construct
QIYPGHGDTNYNGKFKG

SEQ ID NO: 40 - protein, 11 aa, synthetic construct
EGSELGRLFAY

SEQ ID NO: 41 - protein, 10 aa, synthetic construct
SASSSVSFMY

SEQ ID NO: 42 - protein, 7 aa, synthetic construct
LTSNLAS

SEQ ID NO: 43 - protein, 9 aa, synthetic construct
QQWSSNPPT

SEQ ID NO: 44 - protein, 120 aa, synthetic construct
QVQLQQSGAELVKPGASVKISCKASGYAFSSYWMNWVKQRPGKGLEWIGQIYPGHGDTNYNGKFKGKATLTADKSSSTAYMQLSSLTSEDSAVYFCAKEGSELGRLFAYWGQGTLVTVSA

SEQ ID NO: 45 - protein, 106 aa, synthetic construct
QIVLTQSPALMSASPGEKVTMTCSASSSVSFMYWYQQKPRSSPKPWIYLTSNLASGVPARFSGSGSGTSYSLTISSMEAEDAATYYCQQWSSNPPTFGGGTKLEIK

SEQ ID NO: 46 - protein, 5 aa, synthetic construct
DYTIH

SEQ ID NO: 47 - protein, 17 aa, synthetic construct
WFYPGTVSIKYNEKFKD

SEQ ID NO: 48 - protein, 16 aa, synthetic construct
HEHPHYYGDSYDAMGY

SEQ ID NO: 49 - protein, 9 aa, synthetic construct
QHFWGTPYT

SEQ ID NO: 50 - protein, 125 aa, synthetic construct
QVQLQQSGTELVKPGASVKLSCKASGYIFTDYTIHWVKQRSGQGLEWIGWFYPGTVSIKYNEKFKDKATLTADRSSSIVYMELSRLTSEDSGVYFCARHEHPHYYGDSYDAMGYWGQGTSVTVSS

SEQ ID NO: 51 - protein, 107 aa, synthetic construct
DIQMTQSPASLSVSVGETVTITCRASENIYSNLAWYQQKQGKSPQLLVYGATNLADGVPSRFGGSGSGTQYSLKINSLQPEDFGSYYCQHFWGTPYTFGGGTKLEIT

SEQ ID NO: 52 - protein, 5 aa, synthetic construct
DNYLQ

SEQ ID NO: 53 - protein, 14 aa, synthetic construct
PGSGNTYYSDNFTG

SEQ ID NO: 54 - protein, 8 aa, synthetic construct
STVVYFDV

SEQ ID NO: 55 - protein, 5 aa, synthetic construct
SNYAN

SEQ ID NO: 56 - protein, 7 aa, synthetic construct
GTNNRAP

SEQ ID NO: 57 - protein, 7 aa, synthetic construct
WYSNHWV

SEQ ID NO: 58 - protein, 117 aa, synthetic construct
QVQLQQSGPELVKPGASVKISCKASGYIFTDNYLQWVKQRPGQGLEWIGWIFPGSGNTYYSDNFTGKATLTVDKSSITAYMLLSSLTSEDSAVYFCSRSTVVYFDVWGTGTTVTVSS

SEQ ID NO: 59 - protein, 109 aa, synthetic construct
QAVVTQESALTTSPGETVTLTCRSSTGTVTTSNYANWVQEKPDHLFTGLIGGTNNRAPGVPARFSGSLIGDQAALTITGAQTEDEAIYFCALWYSNHWVFGGGTKLTVL

SEQ ID NO: 60 - protein, 5 aa, synthetic construct
DYGMH

SEQ ID NO: 61 - protein, 17 aa, synthetic construct
YISSDSSTIYYADTVKG

SEQ ID NO: 62 - protein, 10 aa, synthetic construct
RAAQGYVMDY

SEQ ID NO: 63 - protein, 11 aa, synthetic construct
KASQSVSDDVA

SEQ ID NO: 64 - protein, 6 aa, synthetic construct
ASNRYT

SEQ ID NO: 65 - protein, 8 aa, synthetic construct
QQDYGSPT

SEQ ID NO: 66 - protein, 119 aa, synthetic construct
EVQLVESGGGLVKPGGSLKLSCAASGFTFSDYGMHWVRQAPEKRLEWVAYISSDSSTIYYADTVKGRFTISRDNAKNTLFLEMTSLRSEDTAMYYCARRAAQGYVMDYWGQGTSVTVSS

SEQ ID NO: 67 - protein, 106 aa, synthetic construct
SIVMTQTPKFLLVSAGDRVTITCKASQSVSDDVAWYQQKPGQSPKLLIYYASNRYTGVPDRFTGSGYGTDFTFTISTVQAEDLAVYFCQQDYGSPTFGGGTKLEIK

SEQ ID NO: 68 - protein, 5 aa, synthetic construct
TYGMS

SEQ ID NO: 69 - protein, 17 aa, synthetic construct
WINTYSGEPTYADEFKG

SEQ ID NO: 70 - protein, 12 aa, synthetic construct
RGYDGYYYTMDY

SEQ ID NO: 71 - protein, 7 aa, synthetic construct
AATNLAD

SEQ ID NO: 72 - protein, 9 aa, synthetic construct
QHFFGAPWT

SEQ ID NO: 73 - protein, 121 aa, synthetic construct
QIQLVQSGPELKKPGETVKISCKASGYTFTTYGMSWVKQAPGKGLKWMAWINTYSGEPTYADEFKGRFAFSLETSVSTAYLQINNLKNEDTATYFCARRGYDGYYYTMDYWGQGTSVTVSS

SEQ ID NO: 74 - protein, 107 aa, synthetic construct
DIQMTQSPASLSASVGETVTITCRASENIYSNLAWYQQKQGKSPQLLVSAATNLADGVPSRFSGSGSGTQFSLKINSLQPEDFGSYYCQHFFGAPWTFGGGTKLEIK

SEQ ID NO: 75 - skipped

SEQ ID NO: 76 - protein, 17 aa, synthetic construct
  VARIANT 1: note = X is R, D, or Y
  VARIANT 3: note = X is N or Y
  VARIANT 5: note = X is G, N, D, or Y
  VARIANT 6: note = X is V or N
  VARIANT 7: note = X is G or Y
  VARIANT 8: note = X is R, G, V, or D
  VARIANT 9: note = X is I or T
  VARIANT 10: note = X is H, S, R, of F
  VARIANT 11: note = X is Y or N
  VARIANT 13: note = X E or Q
  VARIANT 17: note = X is D or G
XIXPXXXXXXXNXKFKX

SEQ ID NO: 77 - skipped

SEQ ID NO: 78 - protein, 14 aa, synthetic construct
  VARIANT 5: note = X is N or S
  VARIANT 6: note = X I or V
  VARIANT 7: note = X is Y or D
  VARIANT 8: note = X is S or N
  VARIANT 9: note = X is N or Y
  VARIANT 10: note = X is L or G
  VARIANT 11: note = X is A, Y, or N
  VARIANT 12: note = X is S, T, or absent
  VARIANT 13: note = X is F or absent
  VARIANT 14: note = X is is L, M, or absent
RASEXXXXXXXXXX

SEQ ID NO: 79-80 - skipped (2 entries)

SEQ ID NO: 81 - protein, 330 aa, synthetic construct [sequence omitted]

SEQ ID NO: 82 - protein, 489 aa, synthetic construct
MTPIVTVLICLRLSLGPRTHVQAGTLPKPTLWAEPGSVITQGSPVTLWCQGILETQEYRLYREKKTAPWITRIPQEIVKKGQFPIPSITWEHTGRYRCFYGSHTAGWSEPSDPLELVVTGAYIKPTLSALPSPVVTSGGNVTLHCVSQVAFGSFILCKEGEDEHPQCLNSQPRTHGWSRAIFSVGPVSPSRRWSYRCYAYDSNSPHVWSLPSDLLELLVLGVSKKPSLSVQPGPIVAPGESLTLQCVSDVSYDRFVLYKEGERDFLQLPGPQPQAGLSQANFTLGPVSRSYGGQYRCSGAYNLSSEWSAPSDPLDILIAGQFRGRPFISVHPGPTVASGENVTLLCQSWGPFHTFLLTKAGAADAPLRLRSIHEYPKYQAEFPMSPVTSAHSGTYRCYGSLSSNPYLLSHPSDSLELMVSGAAETLSPPQNKSDSKAGAANTLSPSQNKTASHPQDYTVENLIRMGIAGLVLVVLGILLFEAQHSQRSL

SEQ ID NO: 83 - protein, 483 aa, synthetic construct
MTPILTVLICLGLSLGPRTHVQAGHLPKPTLWAEPGSVIIQGSPVTLRCQGSLQAEEYHLYRENKSASWVRRIQEPGKNGQFPIPSITWEHAGRYHCQYYSHNHSSEYSDPLELVVTGAYSKPTLSALPSPVVTSGGNVTLQCVSQVAFDGFILCKEGEDEHPQRLNSHSHARGWSWAIFSVGPVSPSRRWSYRCYAYDSNSPYVWSLPSDLLELLVPGVSKKPSLSVQPGPMVAPGESLTLQCVSDVGYDRFVLYKEGERDFLQRPGWQPQAGLSQANFTLGPVSPSHGGQYRCYSAHNLSSEWSAPSDPLDILITGQFYDRPSLSVQPVPTVAPGKNVTLLCQSRGQFHTFLLTKEGAGHPPLHLRSEHQAQQNQAEFRMGPVTSAHVGTYRCYSSLSSNPYLLSLPSDPLELVVSEAAETLSPSQNKTDSTTTSLGQHPQDYTVENLIRMGVAGLVLVVLGILLFEAQHSQRSLQDAAGR

SEQ ID NO: 84 - protein, 439 aa, synthetic construct
MTSILTVLICLGLSLDPRTHVQAGPLPKPTLWAEPGSVITQGSPVTLRCQGSLETQEYHLYREKKTALWITRIPQELVKKGQFPILSITWEHAGRYCCIYGSHTVGLSESSDPLELVVTGAYSKPTLSALPSPVVTSGGNVTIQCDSQVAFDGFILCKEGEDEHPQCLNSHSHARGSSRAIFSVGPVSPSRRWSYRCYGYDSRAPYVWSLPSDLLGLLVPGVSKKPSLSVQPGPVVAPGEKLTFQCGSDAGYDRFVLYKEWGRDFLQRPGRQPQAGLSQANFTLGPVSRSYGGQYTCSGAYNLSSEWSAPSDPLDILITGQIRARPFLSVRPGPTVASGENVTLLCQSQGGMHTFLLTKEGAADSPLRLKSKRQSHKYQAEFPMSPVTSAHAGTYRCYGSLSSNPYLLTHPSDPLELVVSGAAETLSPPQNKSDSKAGE

SEQ ID NO: 85 - protein, 499 aa, synthetic construct
MTLILTSLLFFGLSLGPRTRVQAENLPKPILWAEPGPVITWHNPVTIWCQGTLEAQGYRLDKEGNSMSRHILKTLESENKVKLSIPSMMWEHAGRYHCYYQSPAGWSEPSDPLELVVTAYSRPTLSALPSPVVTSGVNVTLRCASRLGLGRFTLIEEGDHRLSWTLNSHQHNHGKFQALFPMGPLTFSNRGTFRCYGYENNTPYVWSEPSDPLQLLVSGVSRKPSLLTLQGPVVTPGENLTLQCGSDVGYIRYTLYKEGADGLPQRPGRQPQAGLSQANFTLSPVSRSYGGQYRCYGAHNVSSEWSAPSDPLDILIAGQISDRPSLSVQPGPTVTSGEKVTLLCQSWDPMFTFLLTKEGAAHPPLRLRSMYGAHKYQAEFPMSPVTSAHAGTYRCYGSRSSNPYLLSHPSEPLELVVSGATETLNPAQKKSDSKTAPHLQDYTVENLIRMGVAGLVLLFLGILLFEAQHSQRSPPRCSQEANSRKDNAPFRVVEPWEQI

SEQ ID NO: 86 - protein, 299 aa, synthetic construct
MAPWSHPSAQLQPVGGDAVSPALMVLLCLGLSLGPRTHVQAGNLSKATLWAEPGSVISRGNSVTIRCQGTLEAQEYRLVKEGSPEPWDTQNPLEPKNKARFSIPSMTEHHAGRYRCYYYSPAGWSEPSDPLELVVTGFYNKPTLSALPSPVVTSGENVTLQCGSRLRFDRFILTEEGDHKLSWTLDSQLTPSGQFQALFPVGPVTPSHRWMLRCYGSRRHILQVWSEPSDLLEIPVSGAADNLSPSQNKSDSGTASHLQDYAVENLIRMGMAGLILVVLGILIFQDWHSQRSPQAAAGR

SEQ ID NO: 87 - protein, 481 aa, synthetic construct
MTPALTALLCLGLSLGPRTRVQAGPFPKPTLWAEPGSVISWGSPVTIWCQGSLEAQEYQLDKEGSPEPLDRNNPLEPKNKARFSIPSMTQHHAGRYRCHYYSSAGWSEPSDPLELVMTGFYNKPTLSALPSPVVASGGNMTLRCGSQKGYHHFVLMKEGEHQLPRTLDSQQLHSGGFQALFPVGPVTPSHRWRFTCYYYYTNTPRVWSHPSDPLEILPSGVSRKPSLLTLQGPVLAPGQSLTLQCGSDVGYDRFVLYKEGERDFLQRPGQQPQAGLSQANFTLGPVSPSHGGQYRCYGAHNLSSEWSAPSDPLNILMAGQIYDTVSLSAQPGPTVASGENVTLLCQSRGYFDTFLLTKEGAAHPPLRLRSMYGAHKYQAEFPMSPVTSAHAGTYRCYGSYSSNPHLLSFPSEPLELMVSGHSGGSSLPPTGPPSTPASHAKDYTVENLIRMGMAGLVLVFLGILLFEAQHSQRNPQDAAGR

SEQ ID NO: 88 - protein, 650 aa, synthetic construct
MTPILTVLICLGLSLGPRTHVQAGHLPKPTLWAEPGSVITQGSPVTLRCQGGQETQEYRLYREKKTALWITRIPQELVKKGQFPIPSITWEHAGRYRCYYGSDTAGRSESSDPLELVVTGAYIKPTLSAQPSPVVNSGGNVILQCDSQVAFDGFSLCKEGEDEHPQCLNSQPHARGSSRAIFSVGPVSPSRRWWYRCYAYDSNSPYEWSLPSDLLELLVLGVSKKPSLSVQPGPIVAPEETLTLQCGSDAGYNRFVLYKDGERDFLQLAGAQPQAGLSQANFTLGPVSRSYGGQYRCYGAHNLSSEWSAPSDPLDILIAGQFYDRVSLSVQPGPTVASGENVTLLCQSQGWMQTFLLTKEGAADDPWRLRSTYQSQKYQAEFPMGPVTSAHAGTYRCYGSQSSKPYLLTHPSDPLELVVSGPSGGPSSPTTGPTSTSGPEDQPLTPTGSDPQSGLGRHLGVVIGILVAVILLLLLLLLLFLILRHRRQGKHWTSTQRKADFQHPAGAVGPEPTDRGLQWRSSPAADAQEENLYAAVKHTQPEDGVEMDTRSPHDEDPQAVTYAEVKHSRPRREMASPPSPLSGEFLDTKDRQAEEDRQMDTEAAASEAPQDVTYAQLHSLTLRREATEPPPSQEGPSPAVPSIYATLAIH

SEQ ID NO: 89 - protein, 598 aa, synthetic construct
MTPIVTVLICLGLSLGPRTHVQTGTIPKPTLWAEPDSVITQGSPVTLSCQGSLEAQEYRLYREKKSASWITRIRPELVKNGQFHIPSITWEHTGRYGCQYYSRARWSELSDPLVLVMTGAYPKPTLSAQPSPVVTSGGRVTLQCESQVAFGGFILCKEGEDEHPQCLNSQPHARGSSRAIFSVGPVSPNRRWSHRCYGYDLNSPYVWSSPSDLLELLVPGVSKKPSLSVQPGPVVAPGESLTLQCVSDVGYDRFVLYKEGERDLRQLPGRQPQAGLSQANFTLGPVSRSYGGQYRCYGAYNLSSEWSAPSDPLDILITGQIHGTPFISVQPGPTVASGENVTLLCQSWRQFHTFLLTKAGAADAPLRLRSIHEYPKYQAEFPMSPVTSAHAGTYRCYGSLNSDPYLLSHPSEPLELVVSGPSMGSSPPPTGPISTPAGPEDQPLTPTGSDPQSGLGRHLGVVIGILVAVVLLLLLLLLLFLILRHRRQGKHWTSTQRKADFQHPAGAVGPEPTDRGLQWRSSPAADAQEENLYAAVKDTQPEDGVEMDTRAAASEAPQDVTYAQLHSLTLRRKATEPPPSQEGEPPAEPSIYATLAIH

SEQ ID NO: 90 - protein, 631 aa, synthetic construct
MTPALTALLCLGLSLGPRTRVQAGPFPKPTLWAEPGSVISWGSPVTIWCQGSLEAQEYRLDKEGSPEPLDRNNPLEPKNKARFSIPSMTEHHAGRYRCHYYSSAGWSEPSDPLELVMTGFYNKPTLSALPSPVVASGGNMTLRCGSQKGYHHFVLMKEGEHQLPRTLDSQQLHSGGFQALFPVGPVNPSHRWRFTCYYYYMNTPQVWSHPSDPLEILPSGVSRKPSLLTLQGPVLAPGQSLTLQCGSDVGYDRFVLYKEGERDFLQRPGQQPQAGLSQANFTLGPVSPSHGGQYRCYGAHNLSSEWSAPSDPLNILMAGQIYDTVSLSAQPGPTVASGENVTLLCQSWWQFDTFLLTKEGAAHPPLRLRSMYGAHKYQAEFPMSPVTSAHAGTYRCYGSYSSNPHLLSFPSEPLELMVSGHSGGSSLPPTGPPSTPGLGRYLEVLIGVSVAFVLLLFLLLFLLLRRQRHSKHRTSDQRKTDFQRPAGAAETEPKDRGLLRRSSPAADVQEENLYAAVKDTQSEDRVELDSQSPHDEDPQAVTYAPVKHSSPRREMASPPSSLSGEFLDTKDRQVEEDRQMDTEAAASEASQDVTYAQLHSLTLRRKATEPPPSQEGEPPAEPSIYATLAIH

SEQ ID NO: 91 - protein, 448 aa, synthetic construct
MIPTFTALLCLGLSLGPRTDMQAGPLPKPTLWAEPGSVISWGNSVTIWCQGTLEAREYRLDKEESPAPWDRQNPLEPKNKARFSIPSMTEDYAGRYRCYYRSPVGWSQPSDPLELVMTGAYSKPTLSALPSPLVTSGKSVTLLCQSRSPMDTFLLIKERAAHPLLHLRSEHGAQQHQAEFPMSPVTSVHGGTYRCFSSHGFSHYLLSHPSDPLELIVSGSLEGPRPSPTRSVSTAAGPEDQPLMPTGSVPHSGLRRHWEVLIGVLVVSILLLSLLLFLLLQHWRQGKHRTLAQRQADFQRPPGAAEPEPKDGGLQRRSSPAADVQGENFCAAVKNTQPEDGVEMDTRQSPHDEDPQAVTYAKVKHSRPRREMASPPSPLSGEFLDTKDRQAEEDRQMDTEAAASEAPQDVTYARLHSFTLRQKATEPPPSQEGASPAEPSVYATLAIH

SEQ ID NO: 92 - protein, 481 aa, synthetic construct
MTPILMVLICLGLSLGPRTHVQAGTLPKPMLWAEPGSMISEGSPVTLRCQGSLQVQEYRLYKGKKPASWVRRIQQELVKKGYFAIGFITWEHTGQYRCQYYSHSWWSEPSDPLELVVTGAYSKPTLSALPSPVVASGGNVSLQCDSQVTFDGFVLCKEGEDEHPQRLNSQSHARGWSWAVFSVVPVSPSRRWSYRCYGYDSHSPYVWSLPSDLLELLVPGVSKKPSLSVQPGPVVAPGESLTLQCGSDVGYDRFALYKEGERDFLQRPSRQPQAGLSQANFLLGPVSRSHGGQYRCSGAHNLSSEWSAPSDPLDILISGQIRGTPFLSVQPGPTVVSGENVTLLCQSSWQFHAFLLTQAGAADAHLHLRSMYKYPKYQAELSMSPVTSAHAGTYRCYGSLSSNPYLLSVPSDPLELVVSGAVDTLGLSQNNSETKTASHSQDYTVENLIRMGMAGLILVVLGILLFKPQYSQRSLQDAARR

SEQ ID NO: 93 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 94 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 95 - protein, 123 aa, synthetic construct
QVQLVQSGAEVKKPGASVKVSCKASGYTFTDYYMNWVRQAPGQRLEWIGDINPNNGGTSYNQKFKGRATITVDTSASTAYMELSSLRSEDTAVYYCARGGAELTGTYWYFDVWGQGTTVTVSS

SEQ ID NO: 96 - protein, 123 aa, synthetic construct
QVQLVQSGAEVKKPGASVKVSCKASGYTFTDYYMNWVRQAPGQRLEWIGDINPNNGGTSYNQKFKGRATITVDKSASTAYMELSSLRSEDTAVYYCARGGAELTGTYWYFDVWGQGTTVTVSS

SEQ ID NO: 97 - protein, 123 aa, synthetic construct
QVQLVQSGAEVKKPGASVKVSCKASGYTFTDYYMNWVRQAPGQSLEWIGDINPNNGGTSYNQKFKGRATITVDTSASTAYMELSSLRSEDTAVYYCARGGAELTGTYWYFDVWGQGTTVTVSS

SEQ ID NO: 98 - protein, 107 aa, synthetic construct
DIQMTQSPSSLSASVGDRVTITCRASENIYSNLAWYQQKPGKAPKVLVYGATNLADGVPSRFSGSGSGTEYTLTISSLQPEDFATYYCQHFWDSPFTFGGGTKVEIK

SEQ ID NO: 99 - protein, 107 aa, synthetic construct
DIQMTQSPSSLSASVGDRVTITCRASENIYSNLAWYQQKPGKAPKVLVYGATNLADGVPSRFSGSGSGTEFTLTISSLQPEDFATYYCQHFWDSPFTFGGGTKVEIK

SEQ ID NO: 100 - protein, 120 aa, synthetic construct
QVQLVQSGAEVKKPGSSVKVSCKASGYSFTGYSVNWVRQAPGQGLEWIGRINPYNGDIFNNQKFKGRATITVDKSTSTAYMELSSLRSEDTAVYYCARGTTVGGAWFAYWGQGTLVTVSS

SEQ ID NO: 101 - protein, 120 aa, synthetic construct
QVQLVQSGAEVKKPGSSVKVSCKASGYSFTGYSVNWVKQAPGQGLEWIGRINPYNGDIFNNQKFKGRATITVDKSTSTAYMELSSLRSEDTAVYYCARGTTVGGAWFAYWGQGTLVTVSS

SEQ ID NO: 102 - protein, 120 aa, synthetic construct
QVQLVQSGAEVKKPGSSVKVSCKASGYSFTGYSVNWVRQRPGQGLEWIGRINPYNGDIFNNQKFKGRATITVDKSTSTAYMELSSLRSEDTAVYYCARGTTVGGAWFAYWGQGTLVTVSS

SEQ ID NO: 103 - protein, 112 aa, synthetic construct
DIVMTQSPDSLAVSLGERATINCRASESVDSYGYSFLHWYQQKPGQPPKLLIYLASNLESGIPDRFSGSGSGTDFTLTISSLQAEDVAVYYCQQSNEDLMYTFGGGTKVEIK

SEQ ID NO: 104 - protein, 112 aa, synthetic construct
VIVMTQSPDSLAVSLGERATINCRASESVDSYGYSFLHWYQQKPGQPPKLLIYLASNLESGIPDRFSGSGSGTDFTLTISSLQAEDVAVYYCQQSNEDLMYTFGGGTKVEIK

SEQ ID NO: 105 - protein, 124 aa, synthetic construct
QVQLVQSGAEVKKPGSSVKVSCKASGYTFTDYYMNWVRQAPGQGLEWIARIYPGVYRTHYNEKFKDRATLTADKSTSTAYMELSSLRSEDTAVYYCARSGYYGGTYEEDAMDYWGQGTTVTVSS

SEQ ID NO: 106 - protein, 124 aa, synthetic construct
QVQLVQSGAEVKKPGSSVKVSCKASGYTFTDYYMNWVRQAPGQGLEWIARIYPGVYRTHYNEKFKDRVTLTADKSTSTAYMELSSLRSEDTAVYYCARSGYYGGTYEEDAMDYWGQGTTVTVSS

SEQ ID NO: 107 - protein, 107 aa, synthetic construct
DIQMTQSPSSLSASVGDRVTITCRASENIYSNLAWYQQKPGKAPKLLVYAATSLADGVPSRFSGSGSGTDYTLTISSLQPEDFATYYCQNFWDTPYTFGQGTKVEIK

SEQ ID NO: 108 - protein, 107 aa, synthetic construct
DIQMTQSPSSLSASVGDRVTITCRASENIYSNLAWYQQKPGKAPKLLVYAATSLADGVPSRFRGSGSGTDYTLTISSLQPEDFATYYCQNFWDTPYTFGQGTKVEIK

SEQ ID NO: 109 - protein, 120 aa, synthetic construct
QVQLVQSGAEVKKPGSSVKVSCKASGYSFIGYFMNWMRQAPGQGLEWIGRINPYNGDIFYNQKFKGRATITVDKSTSTAYMELSSLRSEDTAVYYCARGITVAAGSFDVWGQGTTVTVSS

SEQ ID NO: 110 - protein, 120 aa, synthetic construct
QVQLVQSGAEVKKPGSSVKVSCKASGYSFIGYFMNWMRQAPGQGLEWIGRINPYNGDIFYNQKFKGRVTITVDKSTSTAYMELSSLRSEDTAVYYCARGITVAAGSFDVWGQGTTVTVSS

SEQ ID NO: 111 - protein, 111 aa, synthetic construct
DIVMTQSPDSLAVSLGERATINCRASESVDNYGNTFMHWYQQKPGQPPKLLIYRASNLESGIPDRFSGSGSKTDFTLTISSLQAEDVAVYYCQQSSDHPLTFGGGTKVEIK

SEQ ID NO: 112 - protein, 111 aa, synthetic construct
DIVMTQSPDSLAVSLGERATINCRASESVDNYGNTFMHWYQQKPGQPPKLLIYRASNLESGIPDRFSGSGSGTDFTLTISSLQAEDVAVYYCQQSSDHPLTFGGGTKVEIK

SEQ ID NO: 113 - protein, 8 aa, synthetic construct
IYPGVYRT

SEQ ID NO: 114 - protein, 6 aa, synthetic construct
ENIYSN

SEQ ID NO: 115 - skipped

SEQ ID NO: 116 - protein, 8 aa, synthetic construct
INPNNGGT

SEQ ID NO: 117 - protein, 5 aa, synthetic construct
ENIYS

SEQ ID NO: 118 - skipped

SEQ ID NO: 119 - protein, 8 aa, synthetic construct
INPYNGDI

SEQ ID NO: 120 - protein, 10 aa, synthetic construct
ESVDSYGYSF

SEQ ID NO: 121 - skipped

SEQ ID NO: 122 - protein, 10 aa, synthetic construct
ESVDNYGNTF

SEQ ID NO: 123 - skipped

SEQ ID NO: 124 - protein, 453 aa, synthetic construct
QVQLVQSGAEVKKPGASVKVSCKASGYTFTDYYMNWVRQAPGQRLEWIGDINPNNGGTSYNQKFKGRATITVDTSASTAYMELSSLRSEDTAVYYCARGGAELTGTYWYFDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 125 - protein, 214 aa, synthetic construct
DIQMTQSPSSLSASVGDRVTITCRASENIYSNLAWYQQKPGKAPKVLVYGATNLADGVPSRFSGSGSGTEYTLTISSLQPEDFATYYCQHFWDSPFTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 126 - protein, 450 aa, synthetic construct
QVQLVQSGAEVKKPGSSVKVSCKASGYSFTGYSVNWVRQAPGQGLEWIGRINPYNGDIFNNQKFKGRATITVDKSTSTAYMELSSLRSEDTAVYYCARGTTVGGAWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 127 - protein, 219 aa, synthetic construct
DIVMTQSPDSLAVSLGERATINCRASESVDSYGYSFLHWYQQKPGQPPKLLIYLASNLESGIPDRFSGSGSGTDFTLTISSLQAEDVAVYYCQQSNEDLMYTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 128 - protein, 454 aa, synthetic construct
QVQLVQSGAEVKKPGSSVKVSCKASGYTFTDYYMNWVRQAPGQGLEWIARIYPGVYRTHYNEKFKDRVTLTADKSTSTAYMELSSLRSEDTAVYYCARSGYYGGTYEEDAMDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 129 - protein, 214 aa, synthetic construct
DIQMTQSPSSLSASVGDRVTITCRASENIYSNLAWYQQKPGKAPKLLVYAATSLADGVPSRFSGSGSGTDYTLTISSLQPEDFATYYCQNFWDTPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 130 - protein, 450 aa, synthetic construct
QVQLVQSGAEVKKPGSSVKVSCKASGYSFIGYFMNWMRQAPGQGLEWIGRINPYNGDIFYNQKFKGRVTITVDKSTSTAYMELSSLRSEDTAVYYCARGITVAAGSFDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 131 - protein, 218 aa, synthetic construct
DIVMTQSPDSLAVSLGERATINCRASESVDNYGNTFMHWYQQKPGQPPKLLIYRASNLESGIPDRFSGSGSGTDFTLTISSLQAEDVAVYYCQQSSDHPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

(19) United States
(12) Patent Application Publication
(10) Pub. No.: US 2026/0167714 A1
(43) Pub. Date: Jun. 18, 2026

(54) ANTI-ILT4 COMPOSITIONS AND METHODS

(71) Applicant: COHERUS BIOSCIENCES, INC., Redwood City, CA (US)

(72) Inventors: Taruna ARORA, Palo Alto, CA (US); Scott L. KLAKAMP, Camarillo, CA (US); Steven E. KAUDER, San Mateo, CA (US); Su-Yi TSENG, San Francisco, CA (US)

(73) Assignee: COHERUS BIOSCIENCES, INC., Redwood City, CA (US)

(21) Appl. No.: 18/852,097

(22) PCT Filed: Mar. 23, 2023
(86) PCT No.: PCT/US2023/064878

§ 371 Date: Sep. 27, 2024

Related U.S. Application Data
(60) Provisional application No. 63/324,544, filed on Mar. 28, 2022.
(60) Provisional application No. 63/374,250, filed on Sep. 1, 2022.

(51) Int. Cl. C07K 16/28; A61K 39/00; A61K 47/68; A61P 37/06

(52) U.S. Cl. CPC C07K 16/2803; A61K 47/6849; A61P 37/06; C07K 16/2818; A61K 2039/54; C07K 2317/14; C07K 2317/24; C07K 2317/33; C07K 2317/52; C07K 2317/565; C07K 2317/73; C07K 2317/76; C07K 2317/92

(57) ABSTRACT

Provided herein are antibodies or antigen-binding fragments thereof that bind to ILT4. Antibodies or antigen-binding fragments thereof are described that antagonize the interaction between ILT4 and HLA-G and/or HLA-A. Also provided herein are methods of treating an autoimmune, neoplastic, or inflammatory disorder by administering antibodies or antigen-binding fragments thereof that bind to ILT4, alone or in combination with an additional therapeutic agent.

### CROSS-REFERENCE TO RELATED APPLICATIONS

This application claims priority benefit of U.S. Provisional Application No. 63/324,544, filed Mar. 28, 2022, and U.S. Provisional Application No. 63/374,250, filed Sep. 1, 2022, the disclosures of which are incorporated herein by reference in their entireties.

### SEQUENCE LISTING

The Sequence Listing filed with this application by EFS, which is entitled “4494-143PCT.xml,” was created on Mar. 23, 2023 and is 130,000 bytes in size, is hereby incorporated by reference in its entirety.

### TECHNICAL FIELD

The present disclosure relates to the field of biotechnology, and more specifically, to anti-ILT4 antibodies and antigen-binding fragments thereof and methods and compositions of anti-ILT4 antibodies and antigen-binding fragments.

### BACKGROUND

Immunoglobulin Like Transcript 4 (ILT4), also called leukocyte immunoglobulin like receptor B2 (LILRB2), is an immunosuppressive molecule predominantly expressed in myeloid cells, including monocytes, macrophages, dendritic cells and granulocytes, tumor cells, and stroma cells. ILT4 is enriched in tumor cells and stromal cells in the tumor microenvironment of certain malignancies. ILT4 expression and signaling in myeloid cells creates a tumor suppressive microenvironment favoring tumor progression. ILT4 has been shown to bind to various ligands including human leukocyte antigen G (HLA)-G, major histocompatibility complex Class I (MHC-I) proteins, e.g., (HLA-A), angiopoietin-like proteins (AngptIs), Nogo66, myelin associated glycoprotein (MAG), oligodendrocyte myelin glycoprotein (OMgp), beta-amyloid, semaphorin 4A (Sema4A), CD1c/d, and complement split products (CSPs). Among the endogenous ligands for ILT4, human leukocyte antigen G (HLA)-G, a nonclassical MHC class I molecule, is expressed by a wide range of tumors. HLA-G has been shown to mediate immunotolerance in cancer through the interaction with ILT4. HLA-G binding to ILT4 can also directly inhibit the function of monocytes, dendritic cells, and neutrophils. As a result, tumor cells can escape innate and adaptive immune response by expressing (HLA)-G. Mechanisms of immunotolerance resulting from the HLA-G/ILT4 interaction include impairment of immune cell proliferation, differentiation, cytotoxicity, cytokine secretion and chemotaxis. The interaction between HLA-G and monocytes due to ILT4 inhibits maturation of human monocyte-derived antigen-presenting cells (APCs) resulting in a reduced expression of MHC class II antigens and co-stimulatory molecules through Stat3 activation. Blocking ILT4 can shift suppressed macrophages (M2) to activated state (M1-like).

Immune checkpoint inhibitors have become a front-line therapy for various malignancies. These include Programmed cell-death protein 1 (PD-1)/programmed cell-death ligand 1 (PD-L1) inhibitors used as both a front-line and second-line therapeutic. The programmed cell death 1 protein, PD-1, is a member of CD28 family and an immunosuppressive receptor expressed on the surfaces of the activated T cells and B cells. The interaction between PD1 and PD-L1 can be blocked to significantly improve the tumor-killing activity of the CD8+ cytotoxic T cells. In particular, PD-1/PD-L1 inhibitors are used in the treatment of patients with non-small-cell lung cancer (NSCLC). For example, compared with traditional therapy, PD-1/PD-L1 inhibitor monotherapy can significantly prolong survival without the level of side effects observed for previous therapeutics used in the treatment of advanced NSCLC. However, some patients harbor tumors that are refractory to PD-1/PD-L1 inhibitors or become refractory to PD-1/PD-L1 inhibitor following treatment and are able to escape the T-cell mediated tumor response.

Thus, there is an unmet need for inhibitors of ILT4 that can block the HLA-G/ILT4 mediated immunosuppressive effects on myeloid cell populations in the tumor microenvironment.

### SUMMARY

The leukocyte immunoglobulin-like receptor (LILR) family contain activating and inhibitory members that can up- or down-regulate immune cell activity. Inhibitory LILR family members include, but are not limited to, LILRB1 (CD85j/ILT2), LILRB2 (CD85d/ILT4), LILRB3 (CD85a/ILT5), LILRB4 (CD85K, ILT3), and LILRB5 (CD85C); and activating LILR family members include LILRA1 (CD851), LILRA2 (CD85 h/ILT1), LILRA4 (CD85g/ILT7), LILRA5 (CD85f), and LILRA6 (CD85b). LILRA3 (CD85e/ILT6) is exclusively expressed in a soluble form.

The present disclosure relates to antibodies or antigen-binding fragments thereof that bind to ILT4, i.e., LILRB2. In some embodiments the antibody or antigen-binding fragment thereof that binds ILT4 comprises a heavy chain variable domain comprising a CDR-H1 comprising an amino acid sequence selected from the group consisting of: DYYMN (SEQ ID NO: 1), GYSVN (SEQ ID NO: 9), DSYMN (SEQ ID NO: 23), GYFMN (SEQ ID NO: 30), SYWMN (SEQ ID NO: 38), DYTIH (SEQ ID NO: 46), DNYLQ (SEQ ID NO: 52), DYGMH (SEQ ID NO: 60), and TYGMS (SEQ ID NO: 68), a CDR-H2 comprising an amino acid sequence selected from the group consisting of: DINPNNGGTSYNQKFKG (SEQ ID NO: 2), RINPYNGDIFNNQKFKG (SEQ ID NO: 10), RIYPGVYRTHYNEKFKD (SEQ ID NO: 17), YINPDNGVTRYNQKFKG (SEQ ID NO: 24), RINPYNGDIFYNQKFKG (SEQ ID NO: 31), QIYPGHGDTNYNGKFKG (SEQ ID NO: 39), WFYPGTVSIKYNEKFKD (SEQ ID NO: 47), PGSGNTYYSDNFTG (SEQ ID NO: 53), YISSDSSTIYYADTVKG (SEQ ID NO: 61), and WINTYSGEPTYADEFKG (SEQ ID NO: 69), and a CDR-H3 comprising an amino acid sequence selected from the group consisting of: GGAELTGTYWYFDV (SEQ ID NO: 3), GTTVGGAWFAY (SEQ ID NO: 11), SGYYGGTYEEDAMDY (SEQ ID NO: 18), EGTITTDLSWFAY (SEQ ID NO: 25), GITVAAGSFDV (SEQ ID NO: 32), EGSELGRLFAY (SEQ ID NO: 40), HEHPHYYGDSYDAMGY (SEQ ID NO: 48), STVVYFDV (SEQ ID NO: 54), RAAQGYVMDY (SEQ ID NO: 62), and RGYDGYYYTMDY (SEQ ID NO: 70). In some embodiments the light chain variable domain comprises a CDR-L1 comprising an amino acid sequence selected from the group consisting of: RASENIYSNLA (SEQ ID NO: 4), RASESVDSYGYSFLH (SEQ ID NO: 12), RASESVDNYGNTFMH (SEQ ID NO: 33), SASSSVSFMY (SEQ ID NO: 41), SNYAN (SEQ ID NO: 55), and KASQSVSDDVA (SEQ ID NO: 63), a CDR-L2 comprising an amino acid sequence selected from the group consisting of: GATNLAD (SEQ ID NO: 5), LASNLES (SEQ ID NO: 13), AATSLAD (SEQ ID NO: 19), ASTNLAD (SEQ ID NO: 26), RASNLES (SEQ ID NO: 34), LTSNLAS (SEQ ID NO: 42), (SEQ ID NO: 53), GTNNRAP (SEQ ID NO: 56), ASNRYT (SEQ ID NO: 64), and AATNLAD (SEQ ID NO: 71), and a CDR-L3 comprising an amino acid sequence selected from the group consisting of: QHFWDSPFT (SEQ ID NO: 6), QQSNEDLMYT (SEQ ID NO: 14), QNFWDTPYT (SEQ ID NO: 20), QHFWDTPYT (SEQ ID NO: 27), QQSSDHPLT (SEQ ID NO: 35), QQWSSNPPT (SEQ ID NO: 43), QHFWGTPYT (SEQ ID NO: 49), WYSNHWV (SEQ ID NO: 57), QQDYGSPT (SEQ ID NO: 65), and QHFFGAPWT (SEQ ID NO: 72).

In some aspects, the antibody or antigen-binding fragment thereof that binds ILT4 comprises a CDR-H1 sequence of: DYYMN (SEQ ID NO: 1), GYSVN (SEQ ID NO: 9), DSYMN (SEQ ID NO: 23), or GYFMN (SEQ ID NO: 30), a CDR-H2 sequence of: DINPNNGGTSYNQKFKG (SEQ ID NO: 2), RINPYNGDIFNNQKFKG (SEQ ID NO: 10), RIYPGVYRTHYNEKFKD (SEQ ID NO: 17), YINPDNGVTRYNQKFKG (SEQ ID NO: 24), or RINPYNGDIFYNQKFKG (SEQ ID NO: 31), or a CDR-H3 sequence of: GGAELTGTYWYFDV (SEQ ID NO: 3), GTTVGGAWFAY (SEQ ID NO: 11), SGYYGGTYEEDAMDY (SEQ ID NO: 18), EGTITTDLSWFAY (SEQ ID NO: 25), or GITVAAGSFDV (SEQ ID NO: 32); and a CDR-L1 sequence of: RASENIYSNLA (SEQ ID NO: 4), RASESVDSYGYSFLH (SEQ ID NO: 12), or RASESVDNYGNTFMH (SEQ ID NO: 33), a CDR-L2 sequence of: GATNLAD (SEQ ID NO: 5), LASNLES (SEQ ID NO: 13), AATSLAD (SEQ ID NO: 19), ASTNLAD (SEQ ID NO: 26), or RASNLES (SEQ ID NO: 34), and a CDR-L3 sequence of: QHFWDSPFT (SEQ ID NO: 6), QQSNEDLMYT (SEQ ID NO: 14), QNFWDTPYT (SEQ ID NO: 20), QHFWDTPYT (SEQ ID NO: 27), or QQSSDHPLT (SEQ ID NO: 35).

In certain aspects, the antibody or antigen-binding fragment thereof comprises: (a) heavy chain variable domain CDRs comprising an amino acid sequence of DYYMN (SEQ ID NO: 1), DINPNNGGTSYNQKFKG (SEQ ID NO: 2), and GGAELTGTYWYFDV (SEQ ID NO: 3); and light chain variable domain CDRs comprising an amino acid sequence of RASENIYSNLA (SEQ ID NO: 4), GATNLAD (SEQ ID NO: 5), and QHFWDSPFT (SEQ ID NO: 6); (b) heavy chain variable domain CDRs comprising an amino acid sequence of GYSVN (SEQ ID NO: 9), RINPYNGDIFNNQKFKG (SEQ ID NO: 10), and GTTVGGAWFAY (SEQ ID NO: 11); and light chain variable domain CDRs comprising an amino acid sequence of RASESVDSYGYSFLH (SEQ ID NO: 12), LASNLES (SEQ ID NO: 13), and QQSNEDLMYT (SEQ ID NO: 14); (c) heavy chain variable domain CDRs comprising an amino acid sequence of DYYMN (SEQ ID NO: 1), RIYPGVYRTHYNEKFKD (SEQ ID NO: 17), and SGYYGGTYEEDAMDY (SEQ ID NO: 18); and light chain variable domain CDRs comprising an amino acid sequence of RASENIYSNLA (SEQ ID NO: 4), AATSLAD (SEQ ID NO: 19), and QNFWDTPYT (SEQ ID NO: 20); (d) heavy chain variable domain CDRs comprising an amino acid sequence of DYYMN (SEQ ID NO: 1), RIYPGVYRTHYNEKFKD (SEQ ID NO: 17), and SGYYGGTYEEDAMDY (SEQ ID NO: 18); and light chain variable domain CDRs comprising an amino acid sequence of RASENIYSNLA (SEQ ID NO: 4), AATSLAD (SEQ ID NO: 19), and QNFWDTPYT (SEQ ID NO: 20); or (e) heavy chain variable domain CDRs comprising an amino acid sequence of DSYMN (SEQ ID NO: 23), YINPDNGVTRYNQKFKG (SEQ ID NO: 24), and EGTITTDLSWFAY (SEQ ID NO: 25); and light chain variable domain CDRs comprising an amino acid sequence of RASENIYSNLA (SEQ ID NO: 4), ASTNLAD (SEQ ID NO: 26), and QHFWDTPYT (SEQ ID NO: 27).

In some embodiments, the antibody or antigen-binding fragment thereof comprises a light chain variable domain comprising an amino acid sequence that is at least 80%, at least 90%, at least 91%, at least 92%, at least 93%, at least 94%, at least 95%, at least 96%, at least 97%, at least 98%, or at least 99% identical to the amino acid sequence: DIQMTQSPASLSISVGETVTITCRASENIYSNLAWYQQKQGKSPQVLVYGATNLADGVPSR FSGSGSGTQYSLKIKSLQSEDFGSYYCQHFWDSPFTFGSGTKLEIK (SEQ ID NO: 8), VIVLTQSPASLAVSLGQRAAISCRASESVDSYGYSFLHWYQQKPGQPPKLLIYLASNLESGIP ARFSGSGSGTDFTLTINPVEADDVATYYCQQSNEDLMYTFGGGTKLEIK (SEQ ID NO: 16), DIQMTQSPASLSVSVGETVTITCRASENIYSNLAWYQQKQGKSPQLLVYAATSLADGVPSR FRGSGSGTQYSLKISSLQSEDFGNYYCQNFWDTPYTFGGGTKLEIK (SEQ ID NO: 22), DIQMTQSPASLSVSVGETVTITCRASENIYSNLAWYQQKQGKSPQLLVYASTNLADGAPAT FSGSGSGTQYSLKINSLQSVDFGSYYCQHFWDTPYTFGGGTKLEIK (SEQ ID NO: 29), DIVLTQSPASLAVSLGQRATISCRASESVDNYGNTFMHWYQQKPGQPPKLLIYRASNLESGI PARFSGSGSKTDFTLTINPVEADDVATYYCQQSSDHPLTFGAGTKLELS (SEQ ID NO: 37), QIVLTQSPALMSASPGEKVTMTCSASSSVSFMYWYQQKPRSSPKPWIYLTSNLASGVPARF SGSGSGTSYSLTISSMEAEDAATYYCQQWSSNPPTFGGGTKLEIK (SEQ ID NO: 45), DIQMTQSPASLSVSVGETVTITCRASENIYSNLAWYQQKQGKSPQLLVYGATNLADGVPSR FGGSGSGTQYSLKINSLQPEDFGSYYCQHFWGTPYTFGGGTKLEIT (SEQ ID NO: 51), QAVVTQESALTTSPGETVTLTCRSSTGTVTTSNYANWVQEKPDHLFTGLIGGTNNRAPGVP ARFSGSLIGDQAALTITGAQTEDEAIYFCALWYSNHWVFGGGTKLTVL (SEQ ID NO: 59), SIVMTQTPKFLLVSAGDRVTITCKASQSVSDDVAWYQQKPGQSPKLLIYYASNRYTGVPDR FTGSGYGTDFTFTISTVQAEDLAVYFCQQDYGSPTFGGGTKLEIK (SEQ ID NO: 67), or DIQMTQSPASLSASVGETVTITCRASENIYSNLAWYQQKQGKSPQLLVSAATNLADGVPSR FSGSGSGTQFSLKINSLQPEDFGSYYCQHFFGAPWTFGGGTKLEIK (SEQ ID NO: 74); and a heavy chain variable domain comprising an amino acid sequence that is at least 80%, at least 90%, at least 91%, at least 92%, at least 93%, at least 94%, at least 95%, at least 96%, at least 97%, at least 98%, or at least 99% identical to the amino acid sequence:

```
  (SEQ ID NO: 7)
EVQLQQSGPELVKPGASVKISCKASGYTFTDYYMNWVKQSHGKSLEWIGDINPNNGGTSY
NQKFKGKATLTVDKSSSTAYMELRSLTSEDSAVYYCARGGAELIGTYWYFDVWGTGTTV
TVSS,
(SEQ ID NO: 15)
DVQLQQSGPELVKPGNSVKISCKAAGYSFTGYSVNWVKERHGKSLEWIGRINPYNGDIFNN
QKFKGKATLTVDKSSSTAHMELRSLTSEDSAVYYCARGTTVGGAWFAYWGQGTLVTVSA,
  (SEQ ID NO: 21)
QVQLKQSGAELVRPGASVKLSCRASGYTFTDYYMNWVKQRPGQGLEWIARIYPGVYRTH
YNEKFKDKATLTAEKSSSTAYMELSSLTSEDSAVYFCARSGYYGGTYEEDAMDYWGQGT
SVTVSS,
  (SEQ ID NO: 28)
EVQLQQSGPELVIPGASVKISCKASGYTFTDSYMNWVKQSHGKSLEWIAYINPDNGVTRYN
QKFKGKATLTVHKSSSTAYMELRSLTSEDSAVYYCAREGTITTDLSWFAYWGQGTLVTVS
A,
(SEQ ID NO: 36)
EVHLQQSGPELVKPGASVKISCKASGYSFIGYFMNWMKQSHGKSLEWIGRINPYNGDIFYN
QKFKGKATLTVDKSSTTAHMDLLSLTSEDFAVYYCARGITVAAGSFDVWGTGTTVTVSS,
  (SEQ ID NO: 44)
QVQLQQSGAELVKPGASVKISCKASGYAFSSYWMNWVKQRPGKGLEWIGQIYPGHGDTN
YNGKFKGKATLTADKSSSTAYMQLSSLTSEDSAVYFCAKEGSELGRLFAYWGQGTLVTVS
A,
  (SEQ ID NO: 50)
QVQLQQSGTELVKPGASVKLSCKASGYIFTDYTIHWVKQRSGQGLEWIGWFYPGTVSIKY
NEKFKDKATLTADRSSSIVYMELSRLTSEDSGVYFCARHEHPHYYGDSYDAMGYWGQGT
SVTVSS,
(SEQ D NO: 58)
QVQLQQSGPELVKPGASVKISCKASGYIFTDNYLQWVKQRPGQGLEWIGWIFPGSGNTYYS
DNFTGKATLTVDKSSITAYMLLSSLTSEDSAVYFCSRSTVVYFDVWGTGTTVTVSSI,
(SEQ ID NO: 66)
EVOLVESGGGLVKPGGSLKLSCAASGFTFSDYGMHWVRQAPEKRLEWVAYISSDSSTIYY
ADTVKGRFTISRDNAKNTLFLEMTSLRSEDTAMYYCARRAAQGYVMDYWGQGTSVTVSS,
or
  (SEQ ID NO: 73)
QIQLVQSGPELKKPGETVKISCKASGYTFTTYGMSWVKQAPGKGLKWMAWINTYSGEPTY
ADEFKGRFAFSLETSVSTAYLQINNLKNEDTATYFCARRGYDGYYYTMDYWGQGTSVTVS
S.
```

In certain embodiments, the antibody or antigen-binding fragment thereof comprises a light chain variable domain comprising an amino acid sequence that is at least 80%, at least 90%, at least 91%, at least 92%, at least 93%, at least 94%, at least 95%, at least 96%, at least 97%, at least 98%, or at least 99% identical to the amino acid sequence set forth in SEQ ID NOs: 8, 16, 22, 29, or 37; and a heavy chain variable domain comprising an amino acid sequence that is at least 91%, at least 92%, at least 93%, at least 94%, at least 95%, at least 96%, at least 97%, at least 98%, or at least 99% identical to the amino acid sequence set forth in SEQ ID NOs: 7, 15, 21, 28, or 36.

In some aspects, the antibody or antigen-binding fragment thereof comprises a light chain variable domain comprising an amino acid sequence set forth in SEQ ID NOs: 8, 16, 22, 29, or 37; and a heavy chain variable domain comprising an amino acid sequence set forth in SEQ ID NOs: 7, 15, 21, 28, or 36.

In certain embodiments, the antibody or antigen-binding fragment thereof comprises: (a) a heavy chain variable domain comprising an amino acid sequence of EVQLQQSGPELVKPGASVKISCKASGYTFTDYYMNWVKQSHGKSLEWIGDINPNNGGTSY NQKFKGKATLTVDKSSSTAYMELRSLTSEDSAVYYCARGGAELTGTYWYFDVWGTGTTV TVSS (SEQ ID NO: 7); and a light chain variable domain comprising an amino acid sequence of DIQMTQSPASLSISVGETVTITCRASENIYSNLAWYQQKQGKSPQVLVYGATNLADGVPSR FSGSGSGTQYSLKIKSLQSEDFGSYYCQHFWDSPFTFGSGTKLEIK (SEQ ID NO: 8); (b) a heavy chain variable domain comprising an amino acid sequence of DVQLQQSGPELVKPGNSVKISCKAAGYSFTGYSVNWVKERHGKSLEWIGRINPYNGDIFNN QKFKGKATLTVDKSSSTAHMELRSLTSEDSAVYYCARGTTVGGAWFAYWGQGTLVTVSA (SEQ ID NO: 15); and a light chain variable domain comprising an amino acid sequence of VIVLTQSPASLAVSLGQRAAISCRASESVDSYGYSFLHWYQQKPGQPPKLLIYLASNLESGIP ARFSGSGSGTDFTLTINPVEADDVATYYCQQSNEDLMYTFGGGTKLEIK (SEQ ID NO: 16); (c) a heavy chain variable domain comprising an amino acid sequence of QVQLKQSGAELVRPGASVKLSCRASGYTFTDYYMNWVKQRPGQGLEWIARIYPGVYRTH YNEKFKDKATLTAEKSSSTAYMELSSLTSEDSAVYFCARSGYYGGTYEEDAMDYWGQGT SVTVSS (SEQ ID NO: 21); and a light chain variable domain comprising an amino acid sequence of DIQMTQSPASLSVSVGETVTITCRASENIYSNLAWYQQKQGKSPQLLVYAATSLADGVPSR FRGSGSGTQYSLKISSLQSEDFGNYYCQNFWDTPYTFGGGTKLEIK (SEQ ID NO: 22); (d) a heavy chain variable domain comprising an amino acid sequence of EVQLQQSGPELVIPGASVKISCKASGYTFTDSYMNWVKQSHGKSLEWIAYINPDNGVTRYN QKFKGKATLTVHKSSSTAYMELRSLTSEDSAVYYCAREGTITTDLSWFAYWGQGTLVTVS A (SEQ ID NO: 28); and a light chain variable domain comprising an amino acid sequence of DIQMTQSPASLSVSVGETVTITCRASENIYSNLAWYQQKQGKSPQLLVYASTNLADGAPAT FSGSGSGTQYSLKINSLQSVDFGSYYCQHFWDTPYTFGGGTKLEIK (SEQ ID NO: 29); or (e) a heavy chain variable domain comprising an amino acid sequence of EVHLQQSGPELVKPGASVKISCKASGYSFIGYFMNWMKQSHGKSLEWIGRINPYNGDIFYN QKFKGKATLTVDKSSTTAHMDLLSLTSEDFAVYYCARGITVAAGSFDVWGTGTTVTVSS (SEQ ID NO: 36); and a light chain variable domain comprising an amino acid sequence of DIVLTQSPASLAVSLGQRATISCRASESVDNYGNTFMHWYQQKPGQPPKLLIYRASNLESGI PARFSGSGSKTDFTLTINPVEADDVATYYCQQSSDHPLTFGAGTKLELS (SEQ ID NO: 37).

In an embodiment, the antibody or antigen-binding fragment thereof comprises: a CDR-H1 comprising an amino acid sequence of X₁X₂X₃X₄N, wherein X₁ is D or G, X₂ is Y or S, X₃ is Y, S, or F, and X₄ is M or V, a CDR-H2 comprising an amino acid sequence of X₁IX₂PX₃X₄X₅X₆X₇X₈X₉NX₁₀KFKX₁₁ (SEQ ID No: 76), wherein X₁ is R, D, or Y; X₂ is N or Y; X₃ is G, N, D, or Y; X₄ is V or N; X₅ is G or Y; X₆ is R, G, V, or D; X₇ is I or T; X₈ is H, S, R, of F; X₉ is Y or N; X₁₀ is E or Q; X₁₁ is D or G, and a CDR-H3 comprising an amino acid sequence of X₁X₂X₃X₄X₅X₆X₇X₈X₉X₁₀X₁₁X₁₂X₁₃X₁₄X₁₅, wherein X₁ is G, S, or E; X₂ is G, T, or I; X₃ is A, T, or Y; X₄ E, V, Y, or I; X₅ is L, G, T, or A; X₆ is T, G, or A; X₇ is G, A, T, or D; X₈ is T, W, Y, L, or S; X₉ is Y, F, E, or S; X₁₀ is W, A, E, or D; X₁₁ is Y, D, F, or V; X₁₂ is F, A, or absent; X₁₃ is D, M, Y, or absent; X₁₄ is V, D, or absent; and X₁₅ is Y or absent.

In an embodiment, the antibody or antigen-binding fragment thereof comprises: a light chain variable domain comprising: a CDR-L1 comprising an amino acid sequence of RASEX₁X₂X₃X₄X₅X₆X₇X₈X₉ X₁₀ (SEQ ID NO: 78), wherein X₁ is N or S; X₂ I or V; X₃ is Y or D; X₄ is S or N; X₅ is N or Y; X₆ is L or G; X₇ or A, Y, or N; X₈ is S, T, or absent; X₉ is F or absent; X₁₀ is L, M, or absent, a CDR-L2 comprising an amino acid sequence of X₁X₂X₃X₄LX₅X₆, wherein X₁ is L, R, G, or A; X₂ is A or S; X₃ is S or T; X₄ is N or S; X₅ is E or A; and X₆ is S or D, and a CDR-L3 comprising an amino acid sequence of X₁QX₂X₃X₄DX₅X₆X₇T, wherein X₁ is absent or Q; X₂ is S, H, N, or Q; X₃ is N, F, or S; X₄ E, W, or S; X₅ is L, S, T, or H; X₆ is M or P; and X₇ is Y, F, or L.

In some embodiments, the antibody or antigen-binding fragment thereof that binds ILT4, comprising heavy chain variable domain CDRs comprising DYYMN (SEQ ID NO: 1), IYPGVYRT (SEQ ID NO: 113), and SGYYGGTYEEDAMDY (SEQ ID NO: 18); and light chain variable domain CDRs comprising ENIYSN (SEQ ID NO: 114), AAT, and QNFWDTPYT (SEQ ID NO: 20). In some embodiments, the antibody or antigen-binding fragment thereof that binds ILT4, comprising heavy chain variable domain CDRs comprising DYYMN (SEQ ID NO: 1), INPNNGGT (SEQ ID NO: 116), and GGAELTGTYWYFDV (SEQ ID NO: 3); and light chain variable domain CDRs comprising ENIYS (SEQ ID NO: 117), GAT, and QHFWDSPFT (SEQ ID NO: 6). In some embodiments, the antibody or antigen-binding fragment thereof that binds ILT4, comprising heavy chain variable domain CDRs comprising GYSVN (SEQ ID NO: 9), INPYNGDI (SEQ ID NO: 119), and GTTVGGAWFAY (SEQ ID NO: 11); and light chain variable domain CDRs comprising ESVDSYGYSF (SEQ ID NO: 120), LAS, and QQSNEDLMYT (SEQ ID NO: 14). In some embodiments, the antibody or antigen-binding fragment thereof that binds ILT4, comprising heavy chain variable domain CDRs comprising GYFMN (SEQ ID NO: 30), INPYNGDI (SEQ ID NO: 119), and GITVAAGSFDV (SEQ ID NO: 32); and light chain variable domain CDRs comprising ESVDNYGNTF (SEQ ID NO: 122), RAS, and QQSSDHPLT (SEQ ID NO: 35).

The present disclosure also relates to methods of treating a subject having an autoimmune, neoplastic, or inflammatory disorder comprising administering the antibodies or antigen-binding fragments that bind to ILT4. In some aspects, the disclosure relates to methods of treating a subject having a cancer. In some embodiments, the cancer is a solid cancer or a liquid cancer. In an aspect, the cancer is a carcinoma or a sarcoma. In particular, median ILT4 expression is particularly high in non-small cell lung cancers (NSCLC) compared to other cancers and NSCLC patients with high ILT4 expression have poor prognosis compared to NSCLC patients with low ILT4 expression.

In some embodiments, the method comprises a) blocking the interaction between ILT4 and HLA-G; b) downregulating ILT4 activity or an HLA-G level; c) removing immune suppression by ILT4 or by HLA-G; d) enhancing monocyte activation; or e) any combination of a) to d). In some embodiments, the method comprises treating or reducing the severity of cancer by reversing or reducing immunosuppression by administering the antibody or antigen-binding fragment thereof that binds to ILT4. In some embodiments, the by reversing or reducing immunosuppression comprises reprogramming myeloid cells by administering the antibody or antigen-binding fragment thereof that binds to ILT4. In some aspects, the present disclosure includes improving T cell activation in a subject by administering an antibody or antigen-binding fragment of the present disclosure. In some aspects, the present disclosure includes improving antigen presentation for T cell priming in a subject by administering an antibody or antigen-binding fragment of the present disclosure. In some aspects, the present disclosure includes promoting macrophage reprogramming in a subject by administering an antibody or antigen-binding fragment of the present disclosure. In some aspects, the present disclosure includes activating dendritic cells in a subject by administering an antibody or antigen-binding fragment of the present disclosure. In some aspects, the present disclosure includes increasing T cell co-stimulation in a subject by administering an antibody or antigen-binding fragment of the present disclosure.

The present disclosure also relates to a method of inducing apoptosis of cancer cells or cancer stem cells comprising contacting the cancer cells or cancer stem cells with an antibody or antigen-binding fragment thereof that binds to ILT4.

Other features and characteristics of the subject matter of this disclosure, as well as the methods of operation, functions of related elements of structure and the combination of parts, and economies of manufacture, will become more apparent upon consideration of the following description and the appended figures, sequence listing, and claims, all of which form a part of this specification.

### BRIEF DESCRIPTION OF DRAWINGS

FIGS. 1A and 1B show levels of TNF-α in supernatants of monocyte-derived macrophages from (A) Donor A and (B) Donor B, respectively. Cells were treated with the indicated concentrations (μg/ml) of the indicated antibodies.

FIGS. 2A and 2B show levels of IL-6 in supernatants of monocyte-derived macrophages from (A) Donor A and (B) Donor B, respectively. Cells were treated with the indicated concentrations (μg/ml) of the indicated antibodies.

FIGS. 3A and 3B show CD206 Mean Fluorescence Intensity for monocyte-derived macrophages from (A) Donor A and (B) Donor B, respectively. Cells were treated with the indicated concentrations (μg/ml) of the indicated antibodies.

FIGS. 4A and 4B show CD209 Mean Fluorescence Intensity for monocyte-derived macrophages from (A) Donor A and (B) Donor B, respectively. Cells were treated with the indicated concentrations (μg/ml) of the indicated antibodies.

FIG. 5 shows cell-associated fluorescence for CHO-S Parental and CHO-S/ILT4 cell lines after incubation with PE-conjugated HLA-G.

FIG. 6 shows cell-associated fluorescence for after preincubation with the indicated mouse anti-human ILT4 antibodies prior to PE-HLA-G incubation. Results measure HLA-G blockade by candidate mouse anti-human ILT4 antibodies.

FIG. 7 shows IC₅₀ curves from an exemplary ILT4 antibody blocking assay using flow cytometry.

FIG. 8 shows blocking activities of humanized anti-ILT4 antibodies on HLA-G binding to ILT4 expression CHO cells. The graph is representative of n=3. Each data point is a mean of triplicates with SD.

FIG. 9 shows blocking activities of humanized anti-ILT4 antibodies on HLA-A binding to ILT4 expression CHO cells. The graph is representative of n=3. Each data point is a mean of triplicates with SD.

FIG. 10 shows the effect of anti-ILT4 mAbs on TNFa production from macrophages. Levels of TNF-α in supernatants of monocyte-derived macrophages from donor 4 and donor 5, respectively. Cells were treated with 4 ug/ml of indicated antibodies and titrated 1:4 with 8 concentration points. Anti-ILT4 antibody and LPS were added 24 hrs before assessment.

FIG. 11 shows the effect of anti-ILT4 mAbs on IL-6 production from macrophages. Levels of TNF-α in supernatants of monocyte-derived macrophages from (A) donor 4 and (B) donor 5, respectively. Cells were treated with 4 ug/ml of indicated antibodies and titrated 1:4 with 8 concentration points. Anti-ILT4 antibody and LPS were added 24 hrs before assessment.

FIG. 12 shows the effect of anti-ILT4 mAbs on cell surface marker expression on macrophages before and after treatment. Flow cytometry data showing Mean Fluorescence Intensity (MFI) as fold increase over control staining for monocyte-derived macrophages. This is data compares expression levels before (medium) and after treatment of 4 ug/ml with anti-ILT4 antibodies for CD206, ILT4, and CD163.

FIG. 13 shows MLR assay to assess T cell activation. M2 macrophages and CD4 T cells were plated at a 1:5 ratio in the presence of anti-ILT4 antibody or anti-ILT4 antibody+anti-PD-1 antibody (Tori). (A) donor 1 and (B) donor2. IgG1 (hIgG1 LALA isotype control).

FIG. 14 shows competition of Anti-ILT4 mAbs binding to ILT4-CHO cells in competition with AF647-Hz45.01-LALA.

FIG. 15 plots the data from FIG. 14 with Hz45.01-LALA vs. 1E1 and HuIgG1-LALA (as a control) on the same graph (left) and Hz45.01-LALA vs. J19 h1 and HuIgG1-LALA (as a control) on the same graph (right).

FIG. 16 shows competition of Anti-ILT4 mAbs binding to ILT4-CHO cells in competition with AF647-Hz156.03-LALA.

FIG. 17 plots the data from FIG. 16 with Hz45.01-LALA vs. 1E1 and HuIgG1-LALA (as a control) on the same graph (left) and Hz45.01-LALA vs. J19 h1 and HuIgG1-LALA (as a control) on the same graph (right).

FIG. 18 shows binding of AF647-Hz45.01-LALA and AF647-Hz156.03-LALA to ILT4-CHO cells at various concentrations (0-10 μg/ml) were incubated with ILT4-CHO cells and analyzed by flow cytometry.

### DETAILED DESCRIPTION

While aspects of the subject matter of the present disclosure may be embodied in a variety of forms, the following description is merely intended to disclose some of these forms as specific examples of the subject matter encompassed by the present disclosure. Accordingly, the subject matter of this disclosure is not intended to be limited to the forms or embodiments so described.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

The present disclosure also relates to a humanized immunoglobulins having binding specificity for ILT4, comprising an antigen binding region of nonhuman origin and at least a portion of an immunoglobulin of human origin. In one aspect, the humanized immunoglobulin includes an antigen binding region of nonhuman origin which binds ILT4 and a constant region derived from a human constant region. In another embodiment, the humanized immunoglobulin which binds ILT4 comprises a complementarity determining region of nonhuman origin and a variable framework region of human origin, and optionally, a constant region of human origin. For example, the humanized immunoglobulin can comprise a heavy chain and a light chain, wherein the light chain comprises a complementarity determining region derived from an antibody of nonhuman origin which binds ILT4 and a framework region derived from a light chain of human origin, and the heavy chain comprises a complementarity determining region derived from an antibody of nonhuman origin which binds ILT4 and a framework region derived from a heavy chain of human origin.

The present disclosure also relates to a humanized immunoglobulin light chain or a humanized immunoglobulin heavy chain. In one aspect, the disclosure relates to a humanized light chain comprising one or more light chain CDRs of nonhuman origin and a human light chain framework region. In another aspect, the present disclosure relates to a humanized immunoglobulin heavy chain comprising one or more heavy chain CDRs of nonhuman origin and a human heavy chain framework region. The CDRs can be derived from a nonhuman immunoglobulin.

Naturally occurring immunoglobulins have a common core structure in which two identical light chains (about 24 kDa) and two identical heavy chains (about 55 or 70 kDa) form a tetramer. The amino-terminal portion of each chain is known as the variable (V) region and can be distinguished from the more conserved constant (C) regions of the remainder of each chain.

Human immunoglobulins can be divided into classes and subclasses, depending on the isotype of the heavy chain. The classes include IgG, IgM, IgA, IgD and IgE, in which the heavy chains are of the gamma (γ), mu (μ), alpha (α), delta (δ) or epsilon (ε) type, respectively. Subclasses include IgG1, IgG2, IgG3, IgG4, IgA1 and IgA2, in which the heavy chains are of the γ1, γ2, γ3, γ4, α1 and α2 type, respectively. Human immunoglobulin molecules of a selected class or subclass may contain either a kappa (κ) or lambda (λ) light chain.

Antibodies can be raised against an appropriate immunogen in a suitable mammal (e.g., a mouse, rat, rabbit or sheep). Antibody-producing cells (e.g., a lymphocyte) can be isolated from, for example, the lymph nodes or spleen of an immunized animal. The cells can then be fused to a suitable immortalized cell (e.g., a myeloma cell line), thereby forming a hybridoma. Fused cells can be isolated employing selective culturing techniques using conventional methods known to a person of ordinary skill in the art. Cells which produce antibodies with the desired specificity can be selected by a suitable assay (e.g., ELISA). Immunoglobulins of nonhuman origin having binding specificity for ILT4 can also be obtained from antibody libraries (e.g., a phage library comprising nonhuman Fab molecules).

In one embodiment, the antigen binding region of the humanized immunoglobulin comprises a CDR of nonhuman origin. In this embodiment, the humanized immunoglobulin having binding specificity for ILT4 comprises at least one CDR of nonhuman origin. For example, CDRs can be derived from the light and heavy chain variable regions of immunoglobulins of nonhuman origin, such that a humanized immunoglobulin includes substantially heavy chain CDR1, CDR2 and/or CDR3, and/or light chain CDR1, CDR2 and/or CDR3, from one or more immunoglobulins of nonhuman origin, and the resulting humanized immunoglobulin has binding specificity for ILT4. In some aspects, all three CDRs of a selected chain are substantially the same as the CDRs of the corresponding chain of a donor, and in some aspects, all three CDRs of the light and heavy chains are substantially the same as the CDRs of the corresponding donor chain.

The portion of the humanized immunoglobulin or immunoglobulin chain which is of human origin (the human portion) can be derived from any suitable human immunoglobulin or immunoglobulin chain. For example, a human constant region or portion thereof, if present, can be derived from the κ or λ light chains, and/or the g (e.g., γ1, γ2, γ3, γ4), μ, α (e.g., α1, α2), δ or ε heavy chains of human antibodies, including allelic variants. A particular constant region (e.g., IgG1), variant or portions thereof can be selected in order to tailor effector function. For example, a mutated constant region (variant) can be used to minimize binding to Fc receptors and/or ability to fix complement.

The term “a” and “an” refers to one or more (i.e., at least one) of the grammatical object of the article. By way of example, “a cell” encompasses one or more cells.

As used herein, the terms “about” and “approximately,” when used to modify an amount specified in a numeric value or range, indicate that the numeric value as well as reasonable deviations from the value known to the skilled person in the art. For example, ±20%, ±10%, or ±5% may be within the intended meaning of the recited value where appropriate. Numerical quantities given are approximate, meaning that the term “around,” “about” or “approximately” can be inferred if not expressly stated.

Concentrations, amounts, and other numerical data may be expressed or presented herein in a range format. It is to be understood that such a range format is used merely for convenience and brevity and thus should be interpreted flexibly to include not only the numerical values explicitly recited as the limits of the range, but also to include all the individual numerical values or sub-ranges encompassed within that range as if each numerical value and sub-range is explicitly recited. As an illustration, a numerical range of “about 0.01 to 2.0” should be interpreted to include not only the explicitly recited values of about 0.01 to about 2.0, but also include individual values and sub-ranges within the indicated range. Thus, included in this numerical range are individual values such as 0.5, 0.7, and 1.5, and sub-ranges such as from 0.5 to 1.7, 0.7 to 1.5, and from 1.0 to 1.5, etc.

Furthermore, such an interpretation should apply regardless of the breadth of the range or the characteristics being described. Additionally, it is noted that all percentages are in weight, unless specified otherwise.

In understanding the scope of the present disclosure, the terms “including” or “comprising” and their derivatives, as used herein, are intended to be open ended terms that specify the presence of the stated features, elements, components, groups, integers, and/or steps, but do not exclude the presence of other unstated features, elements, components, groups, integers and/or steps. The foregoing also applies to words having similar meanings such as the terms “including”, “having” and their derivatives. The term “consisting” and its derivatives, as used herein, are intended to be closed terms that specify the presence of the stated features, elements, components, groups, integers, and/or steps, but exclude the presence of other unstated features, elements, components, groups, integers and/or steps. The term “consisting essentially of,” as used herein, is intended to specify the presence of the stated features, elements, components, groups, integers, and/or steps as well as those that do not materially affect the basic and novel characteristic(s) of features, elements, components, groups, integers, and/or steps. It is understood that reference to any one of these transition terms (i.e. “comprising,” “consisting,” or “consisting essentially”) provides direct support for replacement to any of the other transition term not specifically used. For example, amending a term from “comprising” to “consisting essentially of” or “consisting of” would find direct support due to this definition for any elements disclosed throughout this disclosure. Based on this definition, any element disclosed herein or incorporated by reference may be included in or excluded from the claimed invention.

As used herein, a plurality of compounds, elements, or steps may be presented in a common list for convenience. However, these lists should be construed as though each member of the list is individually identified as a separate and unique member. Thus, no individual member of such list should be construed as a de facto equivalent of any other member of the same list solely based on their presentation in a common group without indications to the contrary.

Furthermore, certain molecules, constructs, compositions, elements, moieties, excipients, disorders, conditions, properties, steps, or the like may be discussed in the context of one specific embodiment or aspect or in a separate paragraph or section of this disclosure. It is understood that this is merely for convenience and brevity, and any such disclosure is equally applicable to and intended to be combined with any other embodiments or aspects found anywhere in the present disclosure, figures, and claims, which all form the application and claimed invention at the filing date. For example, a list of constructs, molecules, method steps, kits, or compositions described with respect to a construct, composition, or method is intended to and does find direct support for embodiments related to constructs, compositions, formulations, and methods described in any other part of this disclosure, even if those method steps, active agents, kits, or compositions are not re-listed in the context or section of that embodiment or aspect.

Unless otherwise specified, a “nucleic acid sequence” encoding a “protein” or “polypeptide” includes all nucleotide sequences that are degenerate versions of each other and thus encode the same amino acid sequence.

The term “exogenous” refers to any material introduced from or originating from outside a cell, a tissue, or an organism that is not produced by or does not originate from the same cell, tissue, or organism in which it is being introduced.

The term “transduced,” “transfected,” or “transformed” refers to a process by which an exogenous nucleic acid is introduced or transferred into a cell. A “transduced,” “transfected,” or “transformed” cell (e.g., mammalian cell) is one that has been transduced, transfected, or transformed with exogenous nucleic acid (e.g., a vector) that includes an exogenous nucleic acid encoding any of the molecules described herein.

The term “nucleic acid” refers to a deoxyribonucleic acid (DNA) or ribonucleic acid (RNA), or a combination thereof, in either a single- or double-stranded form. Unless specifically limited, the term encompasses nucleic acids containing known analogues of natural nucleotides that have similar binding properties as the reference nucleotides. Unless otherwise indicated, a particular nucleic acid sequence also implicitly encompasses complementary sequences as well as the sequence explicitly indicated. In some embodiments of any of the nucleic acids described herein, the nucleic acid is DNA. In some embodiments of any of the nucleic acids described herein, the nucleic acid is RNA.

Modifications can be introduced into a nucleotide sequence by standard techniques known in the art, such as site-directed mutagenesis and polymerase chain reaction (PCR)-mediated mutagenesis. Conservative amino acid substitutions are ones in which the amino acid residue is replaced with an amino acid residue having a similar side chain. Families of amino acid residues having similar side chains have been defined in the art. These families include: amino acids with acidic side chains (e.g., aspartate and glutamate), amino acids with basic side chains (e.g., lysine, arginine, and histidine), non-polar amino acids (e.g., alanine, valine, leucine, isoleucine, proline, phenylalanine, methionine, and tryptophan), uncharged polar amino acids (e.g., glycine, asparagine, glutamine, cysteine, serine, threonine and tyrosine), hydrophilic amino acids (e.g., arginine, asparagine, aspartate, glutamine, glutamate, histidine, lysine, serine, and threonine), hydrophobic amino acids (e.g., alanine, cysteine, isoleucine, leucine, methionine, phenylalanine, proline, tryptophan, tyrosine, and valine). Other families of amino acids include: aliphatic-hydroxy amino acids (e.g., serine and threonine), amide family (e.g., asparagine and glutamine), alphatic family (e.g., alanine, valine, leucine and isoleucine), and aromatic family (e.g., phenylalanine, tryptophan, and tyrosine).

“Affinity” refers to the strength of the sum total of noncovalent interactions between a single binding site of a molecule (e.g., an antibody) and its binding partner (e.g., an antigen or an Fc receptor). Unless indicated otherwise, as used herein, “binding affinity” refers to intrinsic binding affinity (microscopic equilibrium dissociation constant) which reflects a 1:1 interaction between members of a binding pair (e.g., antibody/Fc receptor or antibody and antigen). The affinity of a molecule X for its partner Y can generally be represented by the dissociation constant (K_{D}). Affinity can be measured by common methods known in the art, including those described herein. Specific illustrative and exemplary embodiments for measuring binding affinity are described in the present disclosure and are known by persons skilled in the art.

An “antibody fragment” or “antigen-binding fragment” refers to a molecule other than an intact antibody that comprises a portion of an intact antibody that binds the antigen to which the intact antibody binds. Examples of antibody fragments include but are not limited to Fv, Fab, Fab′, Fab′-SH, F(ab′) 2; diabodies; linear antibodies; single-chain antibody molecules (e.g. scFv); and multispecific antibodies formed from antibody fragments.

The terms “cytotoxin” and “cytotoxic agent” as used herein refer to a substance that inhibits or prevents a cellular function and/or causes cell death or destruction. Cytotoxic agents include, but are not limited to, radioactive isotopes (e.g., At²¹¹, I¹³¹, I¹²⁵, Y⁹⁰, Re¹⁸⁶, Re¹⁸⁸, Sm¹⁵³, Bi²¹², P³², Pb²¹² and radioactive isotopes of Lu); chemotherapeutic agents or drugs (e.g., methotrexate, adriamicin, vinca alkaloids (vincristine, vinblastine, etoposide), doxorubicin, melphalan, mitomycin C, chlorambucil, daunorubicin or other intercalating agents); growth inhibitory agents; enzymes and fragments thereof such as nucleolytic enzymes; antibiotics; toxins such as small molecule toxins or enzymatically active toxins of bacterial, fungal, plant or animal origin, including fragments and/or variants thereof; and the various antitumor or anticancer agents disclosed below.

A “chemotherapeutic agent” is a chemical compound useful in the treatment of cancer. Non-limiting examples of chemotherapeutic agents include alkalyzing or alkylating agents such as thiotepa and cyclosphosphamide (CYTOXAN™); alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethylenethiophosphaoramide and trimethylolomelamine; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, ranimustine; antibiotics such as aclacinomysins, actinomycin, authramycin, azaserine, bleomycins, cactinomycin, calicheamicin, carabicin, caminomycin, carzinophilin, chromomycins, dactinomycin, daunorubicin, detorubicin, 6-diazo-5-oxo-L-norleucine, doxorubicin (Adriamycin), epirubicin, esorubicin, idarubicin, marcellomycin, mitomycins, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine, 5-FU; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; amsacrine; bestrabucil; bisantrene; edatraxate; defofamine; demecolcine; diaziquone; elformithine; elliptinium acetate; etoglucid; gallium nitrate; hydroxyurea; lentinan; lonidamine; mitoguazone; mitoxantrone; mopidamol; nitracrine; pentostatin; phenamet; pirarubicin; podophyllinic acid; 2-ethylhydrazide; procarbazine; PSK®; razoxane; sizofiran; spirogermanium; tenuazonic acid; triaziquone; 2,2′,2″-trichlorotriethylamine; urethan; vindesine; dacarbazine; mannomustine; mitobronitol; mitolactol; pipobroman; gacytosine; arabinoside (“Ara-C”); thiotepa; taxoids, e.g. paclitaxel (TAXOL®, Bristol-Myers Squibb Oncology, Princeton, N.J.) and doxetaxel (TAXOTERE®, Rhône-Poulenc Rorer, Antony, France); chlorambucil; gemcitabine; 6-thioguanine; mercaptopurine; methotrexate; platinum analogs such as cisplatin and carboplatin; platinum; etoposide (VP-16); ifosfamide; mitomycin C; mitoxantrone; vincristine; vinblastine; vinorelbine; navelbine; novantrone; teniposide; daunomycin; aminopterin; xeloda; ibandronate; CPT-11; topoisomerase inhibitor RFS 2000; difluoromethylornithine (DMFO); retinoic acid; esperamicins; capecitabine; and pharmaceutically acceptable salts, acids or derivatives of any of the above. Also included in this definition are anti-hormonal agents that act to regulate or inhibit hormone action on tumors such as anti-estrogens including for example tamoxifen, raloxifene, aromatase inhibiting 4 (5)-imidazoles, 4-hydroxytamoxifen, trioxifene, keoxifene, LY117018, onapristone, and toremifene (Fareston); anti-androgens such as flutamide, nilutamide, bicalutamide, leuprolide, and goserelin; other chemotherapeutic agents such as prednisolone. Pharmaceutically acceptable salts, acids or derivatives of any of the above are included.

A detectable moiety is a compound or composition which may be conjugated directly or indirectly to the antibody. The label may itself be detectable by itself (e.g., radioisotope labels or fluorescent labels) or, in the case of an enzymatic label, may catalyze chemical alteration of a substrate compound or composition which is detectable.

The terms “full length antibody,” “intact antibody,” and “whole antibody” are used herein interchangeably to refer to an antibody having a structure substantially similar to a native antibody structure or having heavy chains that contain an Fc region as defined herein.

The term “treating” or “treatment” as used herein and as is well understood in the art, means an approach for obtaining beneficial or desired results, including clinical results. Beneficial or desired clinical results can include, but are not limited to, alleviation or amelioration of one or more symptoms or conditions, diminishment of extent of disease, stabilizing (i.e. not worsening) the state of disease, delaying or slowing of disease progression, amelioration or palliation of the disease state, diminishment of the reoccurrence of disease, and remission (whether partial or total), whether detectable or undetectable. “Treating” and “treatment” can also mean prolonging survival as compared to expected survival if not receiving treatment. In addition to being useful as methods of treatment, the methods described herein may be useful for the prevention or prophylaxis of disease.

The term “pharmaceutical formulation” refers to a preparation which is in such form as to permit the biological activity of an active ingredient contained therein to be effective, and which contains no additional components which are unacceptably toxic to a subject to which the formulation would be administered.

As used herein, the term “pharmaceutically acceptable” refers to solvents, co-solvents, surfactants, carriers, diluents, excipients, buffers, salts, solvates, hydrates, and/or other components that are compatible with the other ingredients of the formulation and are not deleterious to the recipient thereof. A “pharmaceutically acceptable carrier” refers to an ingredient in a pharmaceutical formulation, other than an active ingredient, which is nontoxic to a subject. A pharmaceutically acceptable carrier may include, but is not limited to, one or more of a solvent, co-solvent, surfactant, diluent, buffer, excipient, stabilizer, or preservative.

As used herein, “buffering agent” refers to a buffer that resists changes in pH by the action of its acid-base conjugate components. The buffering agent may be present in a liquid or solid formulation of the invention. The buffering agent adjusts the pH of the formulation to about 5.0 to about 8.5, to about 5.5 to about 7.5, to about 6.0 to about 6.5, or to a pH of about 6.3. In one aspect, examples of buffering agents that will control the pH in the 5.0 to 7.5 range include acetate, succinate, gluconate, histidine, tartrate, TRIS, citrate, phosphate, maleate, cacodylate, 2-[N-morpholino]ethanesulfonic acid (MES), bis(2-hydroxyethyl)iminotris[hydroxymethyl]methane (Bis-Tris), N-[2-acetamido]-2-iminodiacetic acid (ADA), glycylglycine and other organic acid buffers. In another aspect, the buffering agent herein is histidine or citrate.

A “saccharide” herein is a compound that has a general formula (CH₂O)ₙ and derivatives thereof, including monosaccharides, disaccharides, trisaccharides, polysaccharides, sugar alcohols, reducing sugars, nonreducing sugars, and the like. In one aspect, examples of saccharides herein include glucose, sucrose, trehalose, lactose, fructose, maltose, dextran, erythritol, glycerol, arabitol, sylitol, sorbitol, mannitol, mellibiose, melezitose, raffinose, mannotriose, stachyose, maltose, lactulose, maltulose, glucitol, maltitol, lactitol, iso-maltulose, and the like. A saccharide can be a lyoprotectant. In another aspect, the saccharide herein is a nonreducing disaccharide, such as sucrose.

A “surfactant” herein refers to an agent that lowers surface tension of a liquid. The surfactant can be a nonionic surfactant. In one aspect, examples of surfactants herein include polysorbate (polyoxyethylene sorbitan monolaurate, for example, polysorbate 20 and, polysorbate 80); TRITON (t-Octylphenoxypolyethoxyethanol, nonionic detergent, Union Carbide subsidiary of Dow Chemical Co., Midland Mich.); sodium dodecyl sulfate (SDS); sodium laurel sulfate; sodium octyl glycoside; lauryl-, myristyl-, linoleyl-, or stearyl-sulfobetaine; lauryl-, myristyl-, linoleyl- or stearyl-sarcosine; linoleyl-, myristyl-, or cetyl-betaine; lauroamidopropyl-, cocamidopropyl-, linoleamidopropyl-, myristamidopropyl-, palmidopropyl-, or isostearamidopropyl-betaine (e.g. lauroamidopropyl); myristamidopropyl-, palmidopropyl-, or isostearamidopropyl-dimethylamine; sodium methyl cocoyl-, or disodium methyl oleyl-taurate; sorbitan monopalmitate; and the MONAQUAT series (Mona Industries, Inc., Paterson, N.J.); polyethyl glycol (PEG), polypropylene glycol (PPG), and copolymers of poloxyethylene and poloxypropylene glycol (e.g. Pluronics/Poloxamer, PF68 etc); etc. In another aspect, the surfactant is polysorbate 80.

“Lyophilized” or “Lyophilization” means Freeze-drying that is commonly employed for preserving proteins by removing water from the protein preparation of interest. Freeze-drying, or lyophilization, is a process by which the material to be dried is first frozen and then the ice or frozen solvent is removed by sublimation in a vacuum environment. One or more excipients may be included in pre-lyophilized formulations to enhance stability during the freeze-drying process and/or to improve stability of the lyophilized product upon storage.

A “reconstituted” formulation is one which has been prepared by dissolving a lyophilized protein formulation in a carrier or diluent such that the protein is dispersed in the reconstituted formulation. The reconstituted formulation in suitable for administration (e.g. parenteral administration) to a patient to be treated.

A “lyoprotectant” is a molecule which, when combined with a protein of interest, significantly prevents or reduces chemical and/or physical instability of the protein upon lyophilization and subsequent storage. Exemplary lyoprotectants include sugars/saccharides such as sucrose or trehalose; an amino acid such as monosodium glutamate or histidine; a methylamine such as betaine; a lyotropic salt such as magnesium sulfate; a polyol such as trihydric or higher sugar alcohols, e.g. glycerin, erythritol, glycerol, arabitol, xylitol, sorbitol, and mannitol; propylene glycol; polyethylene glycol; Pluronics; and combinations thereof. The lyoprotectant may be a non-reducing sugar, for example, trehalose or sucrose. A lyoprotectant may be added to the pre-lyophilized formulation in a “lyoprotecting amount” which means that, following lyophilization of the protein in the presence of the lyoprotecting amount of the lyoprotectant, the protein essentially retains its physical and chemical stability and integrity upon lyophilization and storage.

The “carrier or diluent” of interest herein is one which is pharmaceutically acceptable (safe and non-toxic for administration to a human) and is useful for the preparation of a reconstituted formulation. Exemplary diluents include sterile water, bacteriostatic water for injection (BWFI), a pH buffered solution (e.g. phosphate-buffered saline), sterile saline solution, Ringer's solution or dextrose solution.

A “preservative” is a compound which can be added to the diluent to essentially reduce bacterial action in the reconstituted formulation, thus facilitating the production of a multi-use reconstituted formulation, for example. Examples of potential preservatives include octadecyldimethylbenzyl ammonium chloride, hexamethonium chloride, benzalkonium chloride (a mixture of alkylbenzyldimethylammonium chlorides in which the alkyl groups are longchain compounds), and benzethonium chloride. Other types of preservatives include aromatic alcohols such as phenol, butyl and benzyl alcohol, alkyl parabens such as methyl or propyl paraben, catechol, resorcinol, cyclohexanol, 3-pentanol, and m-cresol.

A “bulking agent” is a compound which adds mass to the lyophilized mixture and contributes to the physical structure of the lyophilized cake (e.g. facilitates the production of an essentially uniform lyophilized cake which maintains an open pore structure). Exemplary bulking agents include mannitol, glycine, polyethylene glycol and xorbitol.

As used herein, the term “tonicity modifier” is intended to mean a compound or compounds that can be used to adjust the tonicity of a liquid formulation. Suitable tonicity modifiers include glycerin, lactose, mannitol, dextrose, sodium chloride, magnesium sulfate, magnesium chloride, sodium sulfate, sorbitol, trehalose, sucrose, raffinose, maltose and others known to those or ordinary skill in the art. In one embodiment, the tonicity of the liquid formulation approximates that of the tonicity of blood or plasma.

Unless otherwise specified herein, numbering of amino acid residues in the Fc region or constant region is according to the EU numbering system, also called the EU index, as described in Kabat et al., U.S. Dept. of Health and Human Services, “Sequence of Proteins of Immunological Interest” (1983).

Suitable host cells for cloning or expressing the DNA in the vectors herein are the prokaryote, yeast, or higher eukaryote cells. Suitable prokaryotes for this purpose include eubacteria, such as Gram-negative or Gram-positive organisms, for example, Enterobacteriaceae such as Escherichia, e.g., E. coli, Enterobacter, Erwinia, Klebsiella, Proteus, Salmonella, e.g., Salmonella typhimurium, Serratia, e.g., Serratia marcescans, and Shigella, as well as Bacilli such as B. subtilis and B. licheniformis, Pseudomonas such as P. aeruginosa, and Streptomyces. These examples are illustrative rather than limiting.

Full length antibody, antibody fragments, and antibody fusion proteins can be produced, in some aspects, in bacteria, in particular when glycosylation and Fc effector function are not needed, such as when the therapeutic antibody is conjugated to a cytotoxic agent (e.g., a toxin) and the immunoconjugate by itself shows effectiveness in tumor cell destruction. Full length antibodies have greater half life in circulation. After expression, the antibody is isolated from the E. coli cell paste in a soluble fraction and can be purified through, e.g., a protein A or G column depending on the isotype. Final purification can be carried out similar to the process for purifying antibody expressed e.g., in CHO cells.

In addition to prokaryotes, eukaryotic microbes such as filamentous fungi or yeast are suitable cloning or expression hosts. A number of other genera, species, and strains are commonly available and useful herein, such as Saccharomyces cerevisiae, Schizosaccharomyces pombe; Kluyveromyces hosts such as, e.g., K. lactis, K. fragilis (ATCC 12,424), K. bulgaricus (ATCC 16,045), K. wickeramii (ATCC 24,178), K. waltii (ATCC 56,500), K. drosophilarum (ATCC 36,906), K. thermotolerans, and K. marxianus; yarrowia (EP 402,226); Pichia pastoris (EP 183,070); Candida; Trichoderma reesia (EP 244,234); Neurospora crassa; Schwanniomyces such as Schwanniomyces occidentalis; and filamentous fungi such as, e.g., Neurospora, Penicillium, Tolypocladium, and Aspergillus hosts such as A. nidulans and A. niger.

Examples of useful mammalian host cell lines are monkey kidney CV1 line transformed by SV40 (COS-7, ATCC CRL 1651); human embryonic kidney line (293/293T or 293 cells subcloned for growth in suspension culture; baby hamster kidney cells (BHK, ATCC CCL 10); Chinese hamster ovary cells (CHO); mouse sertoli cells; monkey kidney cells (CV1 ATCC CCL 70); African green monkey kidney cells (VERO-76, ATCC CRL-1587); human cervical carcinoma cells (HELA, ATCC CCL 2); canine kidney cells (MDCK, ATCC CCL 34); buffalo rat liver cells (BRL 3A, ATCC CRL 1442); human lung cells (W138, ATCC CCL 75); human liver cells (Hep G2, HB 8065); mouse mammary tumor (MMT 060562, ATCC CCL51); TRI cells; MRC 5 cells; FS4 cells; and a human hepatoma line (Hep G2).

Host cells are transformed with the above-described expression or cloning vectors and cultured in conventional nutrient media modified as appropriate for inducing promoters, selecting transformants, or amplifying the genes encoding the desired sequences.

In some aspects, the antibody or antigen-binding fragment thereof antagonizes the interaction between ILT4 and HLA-G or HLA-A. In certain embodiments, the antibody or antigen-binding fragment thereof selectively binds to ILT4 relative to other leukocyte immunoglobulin-like receptor family members, for example, ILT1, ILT2, ILT3, ILT5, ILT6, and/or ILT7. In some embodiments, the antibody or antigen-binding fragment thereof specifically binds to human ILT4 with 2-100,000× greater, or at least 2×, 3×, 5×, 10×, 20×, 30×, 40×, 50×, 60×, 100×, 500×, 1000×, 5000×, 10,000×, 13,000×, 14,000×, 15,000×, 20,000×, 50,000×, or 100,000× greater, affinity than the antibody or antigen-binding fragment thereof binds to each of human ILT3 and human ILT2. In some embodiments, the cross-reactivity of the antibody or antigen-binding fragment thereof with other ILT (LILR) family members is absent as measured using surface plasmon resonance.

In some embodiments, the antibody or antigen-binding fragment thereof selectively binds HLA-G over one or more of MHC-I, AngptI, Nogo66, MAG, OMgp, beta-amyloid, Sema4A, CD1c/d, and CSPs. In some embodiments, the antibody or antigen-binding fragment thereof blocks ILT4 binding to HLA-A and/or HLA-G.

In some embodiments, the antibody or antigen-binding fragment thereof binds to ILT4 with an equilibrium disassociation constant (K_{D}) of less than or equal to 1 μM, less than or equal to 1 nM. In some embodiments, the antibody or antigen-binding fragment thereof binds to ILT4 with an equilibrium disassociation constant (K_{D}) of 0.10 nM to 1 nM. In some embodiments, the antibody or antigen-binding fragment thereof binds to ILT4 with an equilibrium disassociation constant (K_{D}) of less than 0.25 nM. In some embodiments, the antibody or antigen-binding fragment thereof binds to ILT4 with an equilibrium disassociation constant (K_{D}) of less than or equal to 1 nM, less than or equal to 0.9 nM, less than or equal to 0.8 nM, less than or equal to 0.7 nM, less than or equal to 0.6 nM, less than or equal to 0.5 nM, less than or equal to 0.4 nM, less than or equal to 0.3 nM, less than or equal to 0.2 nM, less than or equal to 0.1 nM, less than or equal to 0.09 nM, or less than or equal to 0.8 nM, less than or equal to 0.7 nM, less than or equal to 0.6 nM, less than or equal to 0.5 nM, less than or equal to 0.4 nM, less than or equal to 0.3 nM, less than or equal to 0.2 nM, less than or equal to 0.1 nM, less than or equal to 0.09 nM, or less than or equal to 0.07 nM, less than or equal to 0.07 nM, or less than or equal to 0.06 nM, less than or equal to 0.05 nM, or less than or equal to 0.04 nM, less than or equal to 0.03 nM, or less than or equal to 0.02 nM, less than or equal to 0.01 nM, or less than or equal to 0.008 nM. In some embodiments, the antibody or antigen-binding fragment thereof binds to ILT4 with an equilibrium disassociation constant (K_{D}) of 0.5 to 10 pM, 1 to 9 pM, 2 to 8 pM, 3 to 7 pM, or about 1 pM, about 2 pM, about 3 pM, about 4 pM, about 5 pM, about 6 pM, about 7 pM, about 8 pM, about 9 pM, or about 10 pM.

In some embodiments, the antibody or antigen-binding fragment thereof inhibits HLA-G with a potency of about 0.05 to about 0.50 μg/ml, or about about 0.10 to about 0.25 μg/mL. In some embodiments, the antibody or antigen-binding fragment thereof inhibits HLA-G with a potency of less than 0.100 μg/ml.

In some embodiments, the antibody or antigen-binding fragment thereof inhibits HLA-A with a potency of about 0.05 to about 0.50, or about 0.10 to about 0.20 μg/mL. In some embodiments, the antibody or antigen-binding fragment thereof inhibits HLA-A with a potency of less than 0.10 μg/ml.

In some embodiments, the antibody or antigen-binding fragment thereof activates TNF-alpha production with a potency of about 7 ng/ml to about 25 ng/ml or about 12 ng/ml to about 20 ng/mL.

In some embodiments, the antibody or antigen-binding fragment thereof activates IL-6 production with a potency of about 5 ng/ml to about 25 ng/ml, about 6 ng/mL to about 22 ng/ml, or about 8 ng/ml to about 21 ng/mL.

In some embodiments, the antibody comprises a heavy chain constant region selected from the group consisting of IgG1, IgG2, IgG3 or IgG4; and the antibody comprises a light chain constant region selected from the group consisting of kappa and lambda.

In another aspect, the polypeptide may exhibit a reduced affinity to at least one receptor, e.g., FcγI, FcγIIA, or C1q, compared to the polypeptide comprising a wild-type human IgG Fc region.

In still another aspect, the polypeptide comprises a human IgG1, IgG2, IgG3, IgG4, IgA, IgE, or IgM Fc region.

In still another aspect, the polypeptide comprises a human IgG1, IgG2, or IgG4 Fc region.

In some embodiments, the antibody or antigen-binding fragment thereof comprises one or more Fc domains, for example a pair of human Fc domains. In some embodiments, the human Fc domains are human IgG1 Fc domains, human IgG2 Fc domains, human IgG3 Fc domains, or human IgG4 Fc domains. In some embodiments, the human Fc domains are human IgG4 Fc domains. In some embodiments, the human Fc domains each comprise a sequence that is at least 80% identical to SEQ ID NO: 81. In some embodiments, the human Fc domains each comprise a sequence that is at least 90%, 95%, 96%, 97%, 98%, or 99% identical to SEQ ID NO: 81. In some embodiments, the human Fc domains comprise SEQ ID NO: 81. In some embodiments, the human Fc domains include mutations to eliminate glycosylation and/or to reduce Fc-gamma receptor binding. In some embodiments, the human Fc domains comprise the mutation N297Q, N297A, or N297G; in some embodiments the human Fc domains comprise a mutation at position 234 and/or 235, for example L235E, or L234A and L235A (in IgG1), or F234A and L235A (in IgG4); in some embodiments the human Fc domains are IgG2 Fc domains that comprise the mutations V234A, G237A, P238S, H268Q/A, V309L, A330S, or P331S, or a combination thereof (all according to Kabat, EU numbering). In some embodiments, the human Fc domains each comprise human IgG1 constant region mutations L234A/L235A (“LALA”) or human IgG1 constant region mutations L234A/L235A/P329G (“LALAPG”). Non-limiting examples of such mutations are provided in SEQ ID NOs: 93 and 94.

Additional examples of engineered human Fc domains are known to those skilled in the art. Examples of Ig heavy chain constant region amino acids in which mutations in at least one amino acid leads to reduced Fc function include, but are not limited to, mutations in amino acid 228, 233, 234, 235, 236, 237, 239, 252, 254, 256, 265, 270, 297, 318, 320, 322, 327, 329, 330, and 331 of the heavy constant region (according to Kabat, EU numbering). Examples of combinations of mutated amino acids are also known in the art, such as, but not limited to a combination of mutations in amino acids 234, 235, and 331, such as 234, 235, and 329, such as L234F, L235E, and P331S or a combination of amino acids 318, 320, and 322, such as E318A, K320A, and K322A.

Further examples of engineered Fc domains include F243L/R292P/Y300L/V305I/P396 IgG1; S239D/1332E IgG1; S239D/1332E/A330L IgG1; S298A/E333A/K334A; in one heavy chain, L234Y/L235Q/G236W/S239M/H268D/D270E/S298A IgG1, and in the opposing heavy chain, D270E/K326D, A330M/K334E IgG; G236A/S239D/1332E IgG1; K326W/E333S IgG1; S267E/H268F/S324T IgG1; E345R/E430G/S440Y IgG1; N297A or N297Q or N297G IgG1; L235E IgG1; L234A/L235A IgG1; F234A/L235A IgG4; H268Q/V309L/A330S/P331S IgG2; V234A/G237A/P238S/H268A/V309L/A330S/P331S IgG2; M252Y/S254T/T256E IgG1; M428L/N434S IgG1; S267E/L328F IgG1; N325S/L328F IgG1, and the like. In some embodiments, the engineered Fc domain comprises one or more substitutions selected from the group consisting of N297A IgG1, N297Q IgG1, and S228P IgG4.

In one aspect, polypeptides of the present disclosure comprising an Fc variant exhibit decreased affinities to an Fc receptor, e.g., FcγRI, FcγRIIA, FcγRIIIA, relative to an unmodified antibody. In one aspect, polypeptides comprising an Fc variant exhibit affinity for the Fc receptor that is at least 95%, at least 90%, at least 80%, at least 70%, at least 60%, at least 50%, at least 40%, at least 30%, at least 20%, at least 10%, least 5%, or at least 1% less than a than that of a wild type polypeptide.

In one aspect, polypeptides comprising an Fc variant of the present disclosure exhibit, greater than 700-fold reduction in Fcγ binding, or greater than 3,500-fold reduction in Fcγ binding.

In some embodiments, the antibody or antigen-binding fragment thereof comprises a variant Fc region of IgG1, IgG2, IgG3, IgG4, IgA, IgE, or IgM. In certain embodiments the antibody is an aglycosylated antibody with reduced effector functions. In certain embodiments, the variant Fc region of IgG1 comprises (a) an amino acid substitution at position Leu234 with alanine; (b) an amino acid substitution at position Leu235 with alanine; (c) an amino acid substitution at position Pro329 with glycine or arginine; (d) Asn297 with alanine; (e) Asn297 with glutamine; (f) Asn297 with glycine; or (g) any combination of (a) to (f). In certain embodiments, the variant Fc region of IgG2 comprises (g) an amino acid substitution at position Ser228 with proline; (h) an amino acid substitution at position Pro329 with glycine or arginine; or (i) both (g) and (h). In certain embodiments, the variant Fc region of IgG4 comprises (j) an amino acid substitution at position Ser228 with proline; (k) an amino acid substitution at position Leu235 with alanine or glutamate; (1) an amino acid substitution at position Pro329 with glycine or arginine; or (m) any combination of (j) to (1).

The present disclosure also relates to immunoconjugates. In some embodiments, the antibody or antigen-binding fragment thereof is coupled with a chemotherapeutic agent, a cytotoxin, a detectable moiety, a diagnostic agent, or a combination thereof. In other embodiments, the antibody or antigen-binding fragment thereof is administered separately with a chemotherapeutic agent, a cytotoxin, a detectable moiety, a diagnostic agent, or a combination thereof.

The present disclosure also relates to an expression system and a host cell comprising the expression system. The expression system comprises at least one expression vector, including, for example, two or more, or three or more expression vectors. A person of ordinary skill in the art will readily understand that a single or multiple expression vector may be incorporated into a suitable host cell using conventional methods, including, but not limited to, transformation, transfection, or viral infection. The expression system may include one or more nucleic acid sequences encoding a protein comprising an antibody or antigen-binding fragment thereof that binds to ILT4. One or more polypeptide chains comprising the fully formed antibody or antigen-binding fragment thereof that binds to ILT4 may be each expressed by a single vector or separately expressed from two or more vectors within a host cell. For example, the heavy and light chain together comprising the antibody or antigen-binding fragment thereof may be expressed from the same vector or may be expressed from separate vectors.

The present disclosure also relates to a method of manufacturing the antibody or antigen-binding fragment thereof comprising the steps of culturing the host cell comprising the expression system under conditions that allow formation of the antibody or antigen-binding fragment thereof of that binds to ILT4, and recovering the antibody or antigen-binding fragment thereof that binds to ILT4. In some embodiments, the method further comprises purifying the recovered antibody or antigen-binding fragment thereof that binds to ILT4.

The present disclosure also relates to a composition or formulation comprising the antibody or antigen-binding fragment thereof that binds to ILT4 and a carrier including, in some aspects, a pharmaceutically acceptable carrier or a diagnostic carrier. In some embodiments, a pharmaceutical composition may include an additional therapeutic agent. In some embodiments, the additional therapeutic agent is an antagonist of PD-1/PD-L1, TIGIT, or CTLA4. In certain embodiments, the antagonist of PD-L/PD-1 is selected from the group consisting of nivolumab, pembrolizumab, atezolizumab, durvalumab, lambrolizumab, avelumab, and toripalimab. In some aspects, the antagonist of PD-L/PD-1 is toripalimab. The additional therapeutic agent can be, e.g., a chemotherapeutic or a biotherapeutic agent (including but not limited to antibodies or antigen binding fragments thereof that specifically bind to an antigen selected from the group consisting of: PD-L1, PD-L2, CTLA4, BTLA, TIM3, HVEM, GITR, CD27, SIRPα, NKG2A, NKG2C, NKG2E, TSLP, IL10, VISTA, VEGF, EGFR, Her2/neu, VEGF receptors, other growth factor receptors, CD20, CD28, CD40, CD-40L, CD70, CD73, CCR8, OX-40, 4-1BB, and ICOS). The additional therapeutic agent can be selected from the group consisting of STING agonists, poly ADP ribose polymerase (PARP) inhibitors, mitogen-activated protein kinase (MEK) inhibitors, cyclin-dependent kinase (CDK) inhibitors, indoleamine 2,3-dioxygenase (IDO) inhibitors, tryptophan 2,3-dioxygenase (TDO) selective inhibitors, anti-viral compounds, antigens, adjuvants, anti-cancer agents, CTLA-4 pathway antagonists, lipids, liposomes, peptides, cytotoxic agents, chemotherapeutic agents, immunomodulatory cell lines, checkpoint inhibitors, vascular endothelial growth factor (VEGF) receptor inhibitors, topoisomerase II inhibitors, smoothen inhibitors, alkylating agents, anti-tumor antibiotics, anti-metabolites, retinoids, and immunomodulatory agents including but not limited to anti-cancer vaccines.

In some embodiments, the pharmaceutical composition or formulation relates to a liquid formulation of an antibody or antigen-binding fragment thereof that binds to ILT4. In yet a further aspect, this disclosure relates to a lyophilized formulation comprising a mixture of an antibody or antigen-binding fragment thereof that binds to ILT4 and one or more excipients. In certain embodiments, the formulation may further include, but is not limited to, one or more of a buffering agent, a tonicity modifier, a surfactant, a lyoprotectant, a preservative, or bulking agent.

The antibody or antigen-binding fragment thereof that binds to ILT4 in the formulation of the disclosure may have a given concentration, including, for example, a concentration in the range of 1 mg/ml to 200 mg/ml, or at least about 1 mg/mL, at least about 10 mg/mL, at least about 50 mg/mL, at least about 100 mg/mL, at least about 150 mg/mL, at least about 200 mg/mL, or less than 200 mg/ml, or less than 100 mg/ml, or less than 50 mg/ml.

The present disclosure relates to a method of delivering to a subject the antibody or antigen-binding fragment thereof that binds to ILT4, comprising administering the antibody or antigen-binding fragment thereof to the subject via a route of administration selected from parenteral, subcutaneous, intramuscular, intravenous, intrarticular, intrabronchial, intraabdominal, intracapsular, intracartilaginous, intracavitary, intracelial, intracerebellar, intracerebroventricular, intracolic, intracervical, intragastric, intrahepatic, intramyocardial, intraosteal, intrapelvic, intrapericardiac, intraperitoneal, intrapleural, intraprostatic, intrapulmonary, intrarectal, intrarenal, intraretinal, intraspinal, intrathecal, intra-Ommaya, intravitreous, intraocular, intrasynovial, intrathoracic, intrauterine, intravesical, bolus, vaginal, rectal, buccal, sublingual, intranasal, or transdermal.

The present disclosure includes methods for administering an antibody or antigen-binding fragment thereof by introducing the antibody or fragment into the body of a subject. For example, the method comprises piercing the body of the subject with a needle of a syringe, patch, pen, on body injector, or other injection device and injecting the antibody or fragment into the body of the subject, e.g., into the vein, artery, tumor, muscular tissue or subcutis of the subject. For example, an injection device may be a syringe (e.g., pre-filled with the pharmaceutical composition, such as an auto-injector) which, for example, includes a cylinder or barrel for holding fluid to be injected (e.g., comprising the antibody or fragment or a pharmaceutical composition thereof), a needle for piecing skin and/or blood vessels for injection of the fluid; and a plunger for pushing the fluid out of the cylinder and through the needle bore. In an embodiment of the invention, an injection device that comprises an antibody or antigen-binding fragment thereof of the present invention or a pharmaceutical composition thereof is an intravenous (IV) injection device. Such a device includes the antibody or fragment or a pharmaceutical composition thereof in a cannula or trocar/needle which may be attached to a tube which may be attached to a bag or reservoir for holding fluid (e.g., saline; or lactated ringer solution comprising NaCl, sodium lactate, KCl, CaCl₂) and optionally including glucose) introduced into the body of the subject through the cannula or trocar/needle. The present invention provides a vessel (e.g., a plastic or glass vial, e.g., with a cap or a chromatography column, hollow bore needle or a syringe cylinder) comprising any of the antibodies or antigen-binding fragments set forth herein or a pharmaceutical composition thereof comprising a pharmaceutically acceptable carrier.

In some embodiments, the cancer is squamous cell carcinoma, small-cell lung cancer, non-small cell lung cancer, squamous non-small cell lung cancer (NSCLC), nonsquamous NSCLC, glioma, mesothelioma, gastrointestinal cancer, renal cancer (e.g., clear cell carcinoma), ovarian cancer, liver cancer, colorectal cancer, endometrial cancer, kidney cancer (e.g., renal cell carcinoma (RCC)), prostate cancer (e.g., hormone refractory prostate adenocarcinoma), thyroid cancer, neuroblastoma, pancreatic cancer, glioblastoma (glioblastoma multiforme), cervical cancer, stomach cancer, bladder cancer, hepatoma, breast cancer, colon carcinoma, and head and neck cancer (or carcinoma), gastric cancer, germ cell tumor, pediatric sarcoma, sinonasal natural killer, melanoma (e.g., metastatic malignant melanoma, such as cutaneous or intraocular malignant melanoma), bone cancer, skin cancer, uterine cancer, cancer of the anal region, testicular cancer, carcinoma of the fallopian tubes, carcinoma of the endometrium, carcinoma of the cervix, carcinoma of the vagina, carcinoma of the vulva, cancer of the esophagus, cancer of the small intestine, cancer of the endocrine system, cancer of the parathyroid gland, cancer of the adrenal gland, sarcoma of soft tissue, cancer of the urethra, cancer of the penis, solid tumors of childhood, cancer of the ureter, carcinoma of the renal pelvis, neoplasm of the central nervous system (CNS), primary CNS lymphoma, tumor angiogenesis, spinal axis tumor, brain cancer, brain stem glioma, pituitary adenoma, Kaposi's sarcoma, epidermoid cancer, squamous cell cancer, T-cell lymphoma, environmentally-induced cancers including those induced by asbestos, virus-related cancers or cancers of viral origin (e.g., human papilloma virus (HPV-related or -originating tumors)), and hematologic malignancies derived from either of the two major blood cell lineages, i.e., the myeloid cell line (which produces granulocytes, erythrocytes, thrombocytes, macrophages and mast cells) or lymphoid cell line (which produces B, T, NK and plasma cells), such as all types of leukemias, lymphomas, and myelomas, e.g., acute, chronic, lymphocytic and/or myelogenous leukemias, such as acute leukemia (ALL), acute myelogenous leukemia (AML), chronic lymphocytic leukemia (CLL), and chronic myelogenous leukemia (CML), undifferentiated AML (MO), myeloblastic leukemia (M1), myeloblastic leukemia (M2; with cell maturation), promyelocytic leukemia (M3 or M3 variant [M3V]), myelomonocytic leukemia (M4 or M4 variant with eosinophilia [M4E]), monocytic leukemia (M5), erythroleukemia (M6), megakaryoblastic leukemia (M7), isolated granulocytic sarcoma, and chloroma; lymphomas, such as Hodgkin's lymphoma (HL), non-Hodgkin's lymphoma (NHL), B cell hematologic malignancy, e.g., B-cell lymphomas, T-cell lymphomas, lymphoplasmacytoid lymphoma, monocytoid B-cell lymphoma, mucosa-associated lymphoid tissue (MALT) lymphoma, anaplastic (e.g., Ki 1+) large-cell lymphoma, adult T-cell lymphoma/leukemia, mantle cell lymphoma, angio immunoblastic T-cell lymphoma, angiocentric lymphoma, intestinal T-cell lymphoma, primary mediastinal B-cell lymphoma, precursor T-lymphoblastic lymphoma, T-lymphoblastic; and lymphoma/leukaemia (T-Lbly/T-ALL), peripheral T-cell lymphoma, lymphoblastic lymphoma, post-transplantation lymphoproliferative disorder, true histiocytic lymphoma, primary central nervous system lymphoma, primary effusion lymphoma, B cell lymphoma, lymphoblastic lymphoma (LBL), hematopoietic tumors of lymphoid lineage, acute lymphoblastic leukemia, diffuse large B-cell lymphoma, Burkitt's lymphoma, follicular lymphoma, diffuse histiocytic lymphoma (DHL), immunoblastic large cell lymphoma, precursor B-lymphoblastic lymphoma, cutaneous T-cell lymphoma (CTLC) (also called mycosis fungoides or Sezary syndrome), and lymphoplasmacytoid lymphoma (LPL) with Waldenstrom's macroglobulinemia; myelomas, such as IgG myeloma, light chain myeloma, nonsecretory myeloma, smoldering myeloma (also called indolent myeloma), solitary plasmocytoma, and multiple myelomas, chronic lymphocytic leukemia (CLL), hairy cell lymphoma; hematopoietic tumors of myeloid lineage, tumors of mesenchymal origin, including fibrosarcoma and rhabdomyoscarcoma; seminoma, teratocarcinoma, tumors of the central and peripheral nervous, including astrocytoma, schwannomas; tumors of mesenchymal origin, including fibrosarcoma, rhabdomyoscaroma, and osteosarcoma; and other tumors, including melanoma, xeroderma pigmentosum, keratoacanthoma, seminoma, thyroid follicular cancer and teratocarcinoma, hematopoietic tumors of lymphoid lineage, for example T-cell and B-cell tumors, including but not limited to T-cell disorders such as T-prolymphocytic leukemia (T-PLL), including of the small cell and cerebriform cell type; large granular lymphocyte leukemia (LGL) of the T-cell type; a/d T-NHL hepatosplenic lymphoma; peripheral/post-thymic T cell lymphoma (pleomorphic and immunoblastic subtypes); angiocentric (nasal) T-cell lymphoma; cancer of the head or neck, renal cancer, rectal cancer, cancer of the thyroid gland; acute myeloid lymphoma, or a combination or metastasis thereof.

In certain embodiments, the cancer is solid tumor. In other embodiments, the cancer is hematologic cancer. In certain embodiments, the cancer is metastatic. In some embodiments, the cancer is relapsed. In other embodiments, the cancer is refractory. In yet other embodiments, the cancer is relapsed and refractory. In some embodiments, the cancer is anaplastic astrocytoma, astrocytoma, bladder cancer, bone cancer, brain cancer, breast cancer (e.g., characterized by a mutation in BRCA1 and/or BRCA2), carcinoid cancer, cervical cancer, chondrosarcoma, choroid plexus papilloma, colorectal cancer, endometrial cancer, ependymoma, esophagus cancer, Ewing's sarcoma, gall bladder cancer, gastric cancer, glioblastoma, head and neck cancer, hepatoblastoma, hepatocellular carcinoma, idiopathic myelfibrosis, kidney cancer, leukemia, liver cancer, lung cancer (e.g., non-small cell lung cancer), lymphoma, medulloblastoma, melanoma, meningioma, Merkel cell cancer, mesothelioma, multiple myeloma, neuroblastoma, oligodendroglioma, osteosarcoma, ovarian cancer, pancreatic cancer, polycythemia vera, primitive neuroectodermal tumor, prostate cancer, renal cell cancer, renal transitional cell cancer, retinoblastoma, rhabdoid tumor of the kidney, rhabdomyosarcoma, salivary gland cancer, sarcoma, small intestine cancer, soft tissue sarcoma, squamous cell carcinoma (e.g., cutaneous squamous cell carcinoma), synovial sarcoma, thrombocythemia, thyroid cancer, uterine cancer, vestibular schwannoma, or Wilm's tumor. In an embodiment of the invention, the cancer is metastatic cancer, e.g., of the varieties described above.

In an embodiment of the invention, the cancer is a myeloid-rich tumor (e.g., mesothelioma, kidney cancer, lymphoma, sarcoma, melanoma, head & neck cancer, breast cancer, bladder cancer, gastric cancer, ovarian cancer or thyroid cancer. Since ILT4 is expressed primarily by myeloid cells and granulocytes, and myeloid cell infiltration into tumors is generally associated with poor prognosis due to the immunosuppressive effects of these cells that can antagonize anti-tumor responses by T-cells, treatment with an anti-ILT4 antibody or antigen-binding fragment of the present disclosure will benefit subjects with a high myeloid or immunosuppressive myeloid cell infiltration.

In certain embodiments, the cancer is lung cancer, liver cancer, a NSCLC, ovarian cancer, cervical cancer, skin cancer, bladder cancer, colon cancer, breast cancer, pancreatic cancer, glioblastoma, glioma, renal carcinoma, stomach cancer, gastric cancer, esophageal cancer, oral squamous cell cancer, or head/neck cancer.

The present invention includes ELISA assays (enzyme-linked immunosorbent assay) incorporating the use of an antibody or antigen-binding fragment thereof disclosed herein. For example, such a method, for determining if a sample contains ILT4 or a fragment thereof, comprises the following steps:
(a) coating a substrate (e.g., surface of a microtiter plate well, e.g., a plastic plate) with anti-ILT4 antibody (e.g., fully human antibodies such as antagonist fully human antibodies) or antigen-binding fragment thereof;
(b) applying a sample to be tested for the presence of ILT4 to the substrate;
(c) washing the substrate, so that unbound material in the sample is removed;
(d) applying detectably labeled antibodies (e.g., enzyme-linked antibodies) which are also specific to the ILT4 antigen;
(e) washing the substrate, so that the unbound, labeled antibodies are removed;
(f) detecting the label on the antibodies; if the labeled antibodies are enzyme linked, apply a chemical which is converted by the enzyme into a detectable, e.g., fluorescent, signal; and
(g) detection of the label, e.g., associated with the substrate, indicates the presence of the ILT4 protein.

The present invention also includes Surface Plasmon Resonance (SPR) assays incorporating the use of an antibody or antigen-binding fragment thereof disclosed herein. In some aspects, the present disclosure includes evaluation of antibody binding activity, affinity, and cross reactivity and comprise the following steps:
(a) preparing one or more capture surfaces by immobilizing anti-Fc molecules on the one or more capture surfaces;
(b) flowing a solution comprising anti-ILT4 antibodies over the one or more capture surfaces to immobilize anti-ILT4 on the one or more surfaces;
(c) flowing an analyte over the capture surface following step (b) to measure association of the analyte to the anti-ILT4 antibodies; and
(d) flowing buffer over the capture surface following step (c) to measure dissociation of analyte from the anti-ILT4 antibodies;
(e) flowing a regeneration solution over the one or more capture surfaces in step (d) to remove any remaining analyte/anti-ILT4 complexes or unbound anti-ILT4 antibodies from the surface.

Binding affinity may be determined by dividing the rate of dissociation by the rate of association. In some embodiments, may include flowing one or more additional analytes over the capture surface.

The present invention also comprises a competitive binding assay between ILT4 antibodies and one or more analytes comprising the steps:
(a) preparing one or more capture surfaces by capturing a first ligand to the one or more capture surfaces;
(b) preparing a solution comprising anti-ILT4 antibodies and a second analyte;
(c) flowing the solution comprising the anti-ILT4 antibodies and the second analyte over the capture surface prepared in step (a); and
(d) measuring a response.

In some embodiments the capture surface may be activated, for example, with EDC/NHS (N-ethyl-N′-(3-dimethylaminopropyl) carbodiimide hydrochloride and N-hydroxysuccinimide (Biacore GE Healthcare) and deactivated, for example, using ethanolamine (Biacore GE Healthcare), according to manufacturer's instructions. In some embodiments capture surface is coated with, for example, avidin, streptavidin, neutravidin, or a derivatives thereof to immobilize a biotinylated agent.

In an embodiment of the invention, the labeled antibody or antigen-binding fragment thereof is labeled with peroxidase which reacts with ABTS (e.g., 2,2′-azino-bis(3-ethylbenzthiazoline-6-sulphonic acid)) or 3,3′,5,5′-Tetramethylbenzidine to produce a color change which is detectable. Alternatively, the labeled antibody or fragment is labeled with a detectable radioisotope (e.g., 3H) which can be detected by scintillation counter in the presence of a scintillant.

An anti-ILT4 antibody (e.g., fully human antibodies such as antagonist fully human antibodies) or antigen-binding fragment thereof of the invention may be used in a Western blot or immune-protein blot procedure. Such a procedure forms part of the present invention and includes e.g.: (1) providing a membrane or other solid substrate comprising a sample to be tested for the presence of ILT4 or fragment thereof, e.g., optionally including the step of transferring proteins from a sample to be tested for the presence of ILT4 (e.g., from a PAGE or SDS-PAGE electrophoretic separation of the proteins in the sample) onto a membrane or other solid substrate (e.g., using methods known in the art (e.g., semi-dry blotting or tank blotting)); and contacting the membrane or other solid substrate to be tested for the presence of bound ILT4 or a fragment thereof with an anti-ILT4 antibody or antigen-binding fragment thereof of the invention; (2) washing the membrane one or more times to remove unbound anti-ILT4 antibody or fragment and other unbound substances; and (3) detecting the bound anti-ILT4 antibody or fragment.

Such a membrane may take the form, for example, of a nitrocellulose or vinyl-based (e.g., polyvinylidene fluoride (PVDF)) membrane to which the proteins to be tested for the presence of ILT4 in a non-denaturing PAGE (polyacrylamide gel electrophoresis) gel or SDS-PAGE (sodium dodecyl sulfate polyacrylamide gel electrophoresis) gel have been transferred (e.g., following electrophoretic separation in the gel). Before contacting the membrane with the anti-ILT4 antibody or fragment, the membrane is optionally blocked, e.g., with non-fat dry milk or the like so as to bind non-specific protein binding sites on the membrane.

Detection of the bound anti-ILT4 antibody or fragment indicates that the ILT4 protein is present on the membrane or substrate and in the sample. Detection of the bound antibody or fragment may be by binding the antibody or fragment with a secondary antibody (an anti-immunoglobulin antibody) which is detectably labeled and, then, detecting the presence of the secondary antibody label.

The anti-ILT4 antibodies (e.g., fully human antibodies such as antagonist fully human antibodies) and antigen-binding fragments thereof disclosed herein may also be used for immunohistochemistry. Such a method forms part of the present invention and comprises, e.g., (1) contacting cells (e.g., myeloid lineage cells such as monocytes, macrophages or dendtritic cells) to be tested for the presence of ILT4 protein with an anti-ILT4 antibody or antigen-binding fragment thereof of the invention; and (2) detecting the antibody or fragment on or in the cells.

If the antibody or fragment itself is detectably labeled, it can be detected directly. Alternatively, the antibody or fragment may be bound by a detectably labeled secondary antibody wherein the label is then detected.

### EXAMPLES

The following examples are intended to exemplify the present disclosures and are not limitations of the claimed invention. All molecules, compositions, methods, assays, and results disclosed in the examples form part of the present invention.

### Example 1

Creation and Selection of Mouse anti-human Immunoglobulin-Like Transcript 4 (ILT4) antibodies: Mice were immunized with human ILT4 and screened for high titers of anti-ILT4 antibodies. A primary screen was conducted for hybridomas producing antibodies that bind to ILT4 and that block the ILT4/HLA-G interaction using flow cytometry. Additional screening was conducted to confirm binding to ILT4 and characterize ILT4 binding specificity. Antibodies selected following the secondary screen were purified and further tested for affinity to ILT4 and potency against the ILT4/HLA-G binding. Antibodies from the secondary screen with the highest ILT4 affinity and ILT4/HLA-G blocking activity were selected as leads for further characterization and humanization. Antibodies selected from the secondary screen were confirmed to have the ability to block ILT4 binding to both HLA-G and HLA-A by flow cytometry and SPR.

### Example 2

Antibody Humanization: Design of humanized VH and VL amino acid sequences was carried out as follows according to the procedure described in Tsurushita et al. (Tsurushita et al. 2005. Design of humanized antibodies: From anti-Tac to Zenapax. Methods 36:69-83), which is incorporated by reference herein in its entirety. First, a three-dimensional molecular model of the mouse monoclonal antibody (mouse-mAb) variable regions was constructed. Second, the framework amino acid residues important for the formation of the CDR structure or necessary for antigen binding were predicted using the molecular model. In parallel, cDNA-derived human VH and VL amino acid sequences with high homology to the mouse-mAb VH and VL amino acid sequences, respectively, were selected. Lastly, CDR sequences together with framework amino acid residues important for the formation of the antigen binding site were grafted from the mouse-mAb VH and VL into the corresponding selected human framework sequences.

Human VH sequences homologous to the mouse-mAb VH frameworks were searched for within the GenBank database and the VH sequence encoded with no somatic hypermutations in the frameworks was chosen as an acceptor for humanization. The CDR sequences of mouse-mAb VH were first transferred to the corresponding positions of the VH from the GenBank sequence. Next, at the framework positions where the three-dimensional model of the mouse-mAb variable regions indicated significant contact with the CDRs, amino acid residues of the VH from the GenBank sequence were replaced with the corresponding residues of mouse-mAb VH.

Human VL region sequences homologous to the mouse-mAb VL framework sequences were next searched within the GenBank database and the VL sequence encoded with no somatic hypermutations in the frameworks was chosen as an acceptor for humanization. CDR sequences of mouse-mAb VL were first transferred to the corresponding positions of the GenBank VL. Next, framework positions where the three-dimensional model of the mouse-mAb variable regions indicated significant contact with the CDRs, amino acid residues of the GenBank VL were replaced with the corresponding residues of the mouse-mAb VL as with the VH regions described above.

### Example 3

Macrophage repolarization by mouse anti-human Immunoglobulin-Like Transcript 4 (ILT4) antibodies: Known ILT4 antibodies were tested in a macrophage repolarization assay developed to confirm activity of known ILT4 antibodies as a control. Human monocytes were differentiated toward M2 macrophages for 6 days, then stimulated with lipopolysaccharide (LPS) and the ILT4 antibody to be assayed using ELISA.

Mouse anti-human Immunoglobulin-Like Transcript 4 (ILT4) antibodies (L45S4, L77S2, L156S3, L161S and L180S4) and control antibodies (anti-KLH IgG4 negative control; and the control antibody J19 h1 as a positive control) were used in a human macrophage repolarization assay. Macrophage repolarization was measured by Meso Scale Discovery (MSD) assay of secreted cytokines using a MSD Plate Reader (MESO Sector S 600 from Meso Scale Diagnostics LLC) and flow cytometry analysis of cell surface proteins using a Flow Cytometer (Attune NXT from ThermoFisher). Human monocyte-derived macrophages of the suppressive M2 phenotype were made by derivation in the presence of M-CSF. These cells were stimulated with LPS and the phenotype evaluated by MSD analysis and flow cytometry.

Generation of Monocyte-Derived Macrophages: PBMCs were isolated from fresh buffy coats from two different blood donors using standard density centrifugation. Cells were re-suspend with buffer and counted. Cell density was then adjusted to 5×10⁷ cells/ml in buffer. Isolated monocytes from PBMCs were centrifuged and resuspended in culture medium. Density was adjusted to 0.5×10⁶ cells/ml, at 30 ml differentiation medium and cells were plated. After three days, half-volume of additional differentiation medium was added. Cells were cultured for an additional three days.

Macrophage Stimulation and Treatment With Antibody: Cells were plated using growth medium. Serial dilutions of test antibodies were prepared in duplicate wells to plates for a final concentration ranging from 30 μg/ml to 0.03 ng/ml. Subsequently, LPS was was added to a final concentration of 100 ng/ml.

Macrophage Phenotype Analysis: Human macrophages of the suppressive M2 phenotype were made, then stimulated with LPS in the presence of the ILT4 antibodies or control antibodies. After 2 days LPS stimulation in the presence of the ILT4 antibodies or controls, plates were centrifuged and supernatants collected for quantitation of the proinflammatory cytokines TNF-α and IL-6 by MSD analysis.

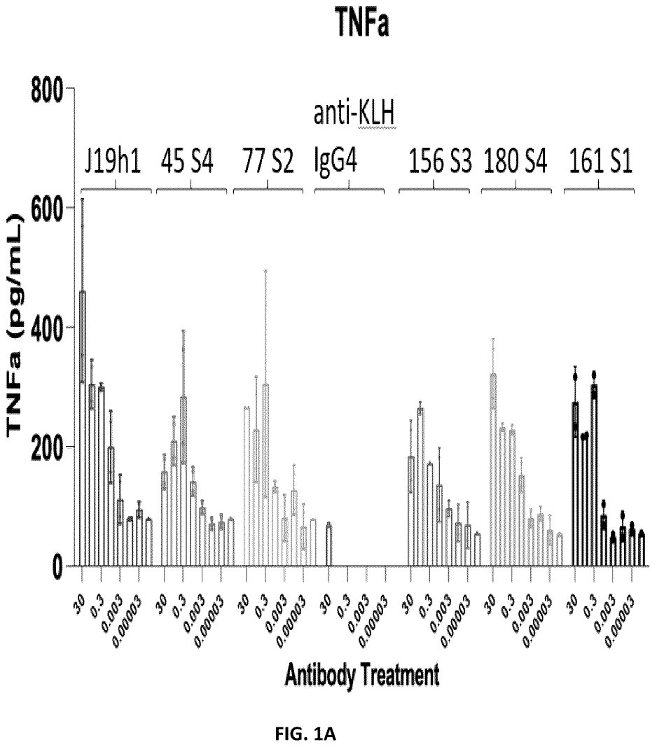

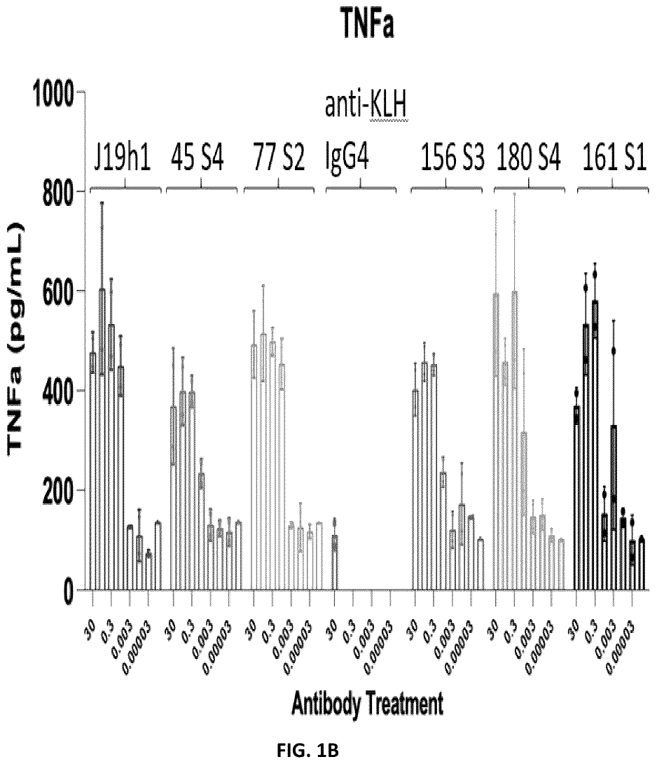

Mesoscale Discovery Assay of Cytokine Secretion: To determine the effect of mouse anti-human ILT4 antibodies on macrophage polarity, supernatants were analyzed by MSD. For both blood donors, all of the ILT4 antibodies increased TNF-α secretion over the control (FIGS. 1A and 1B). The maximum effect ranged from a 4- to 5-fold increase over the anti-KLH IgG4 negative control. The control antibody increased TNF-α secretion 8-fold over the negative control.

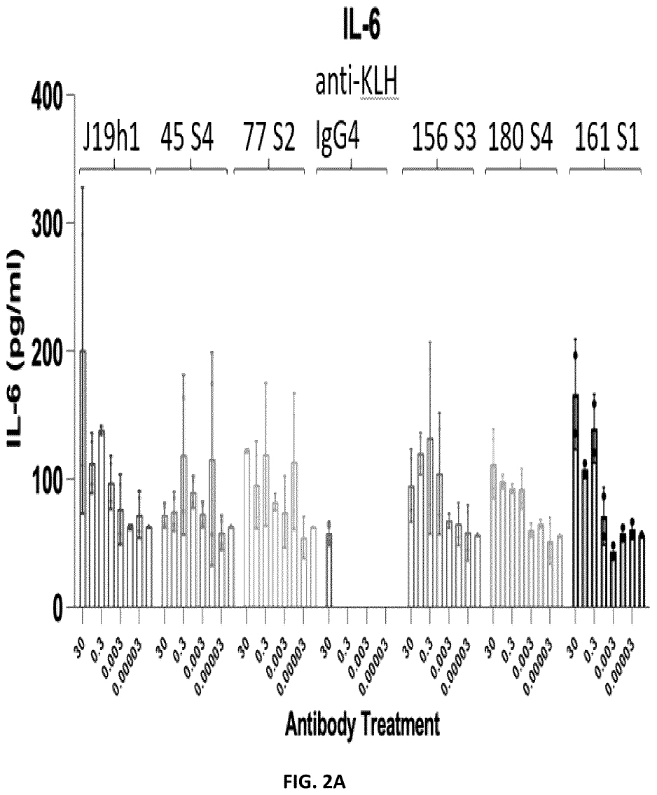

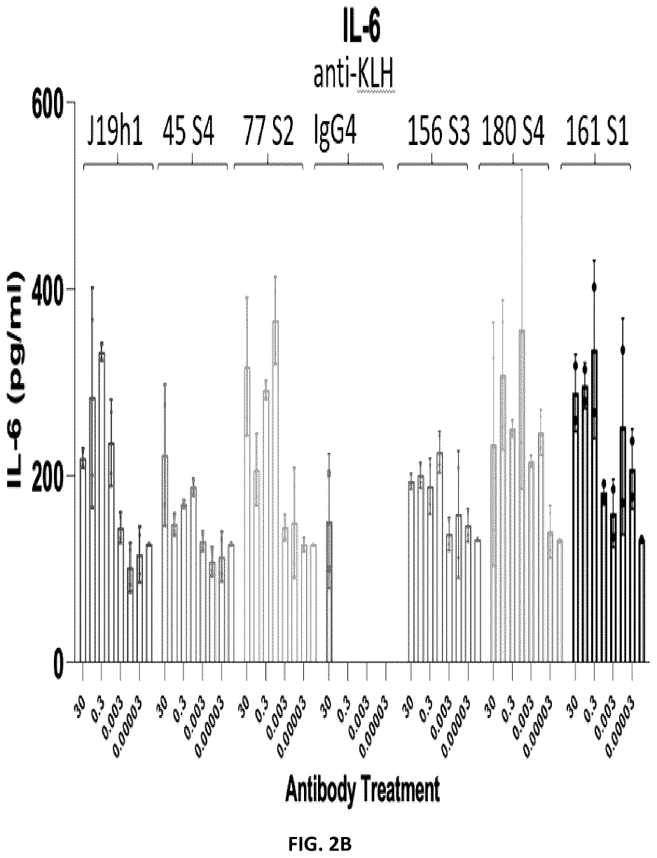

For both blood donors, all of the ILT4 antibodies tested also increased IL-6 secretion by two-fold over the control at the highest concentration (FIGS. 2A and 2B). The control antibody increased IL-6 secretion by 3-fold for Donor A and 2-fold for Donor B.

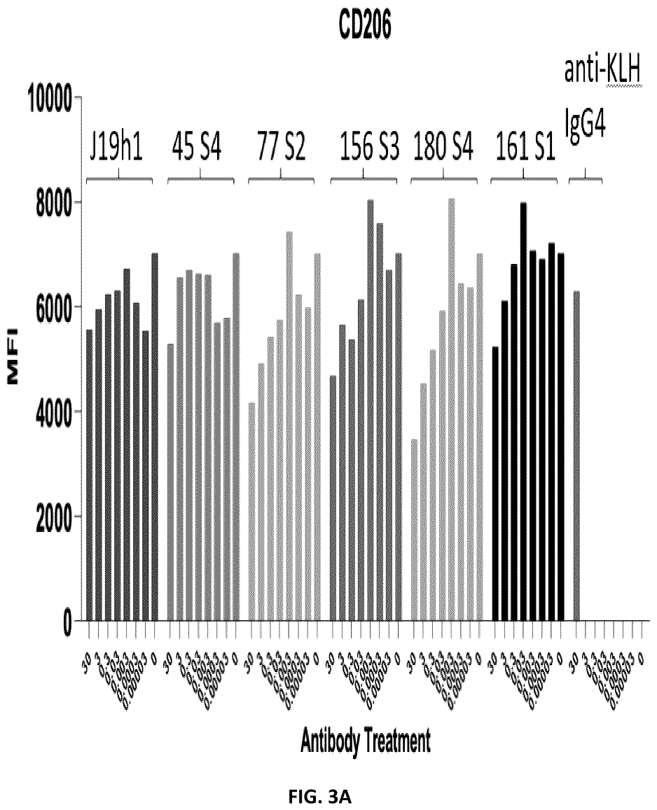

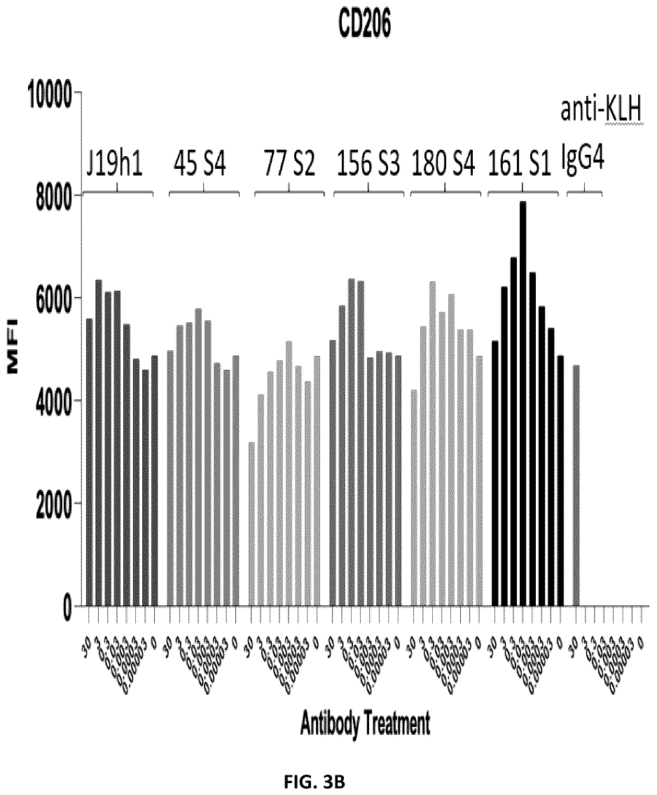

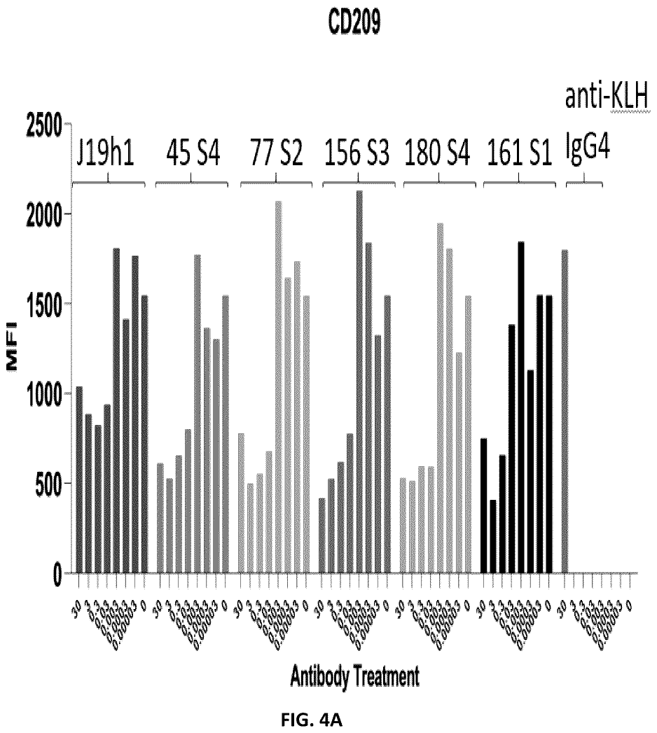

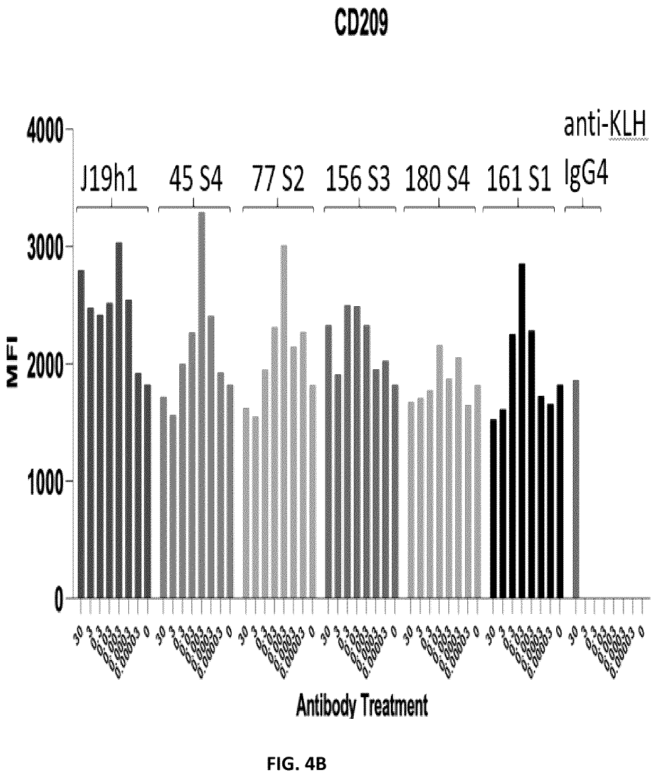

Flow Cytometry Analysis of Surface Marker Expression by Macrophages: Expression of polarity-indicating cell surface proteins was assessed by flow cytometry. Mean fluorescence intensity (MFI) of staining for CD206 (FIGS. 3A and 3B), and CD209 (FIGS. 4A and 4B) were determined. ILT4 antibodies did not induce a change in macrophage surface protein expression from M1-type to M2-type proteins. This was observed for both the control antibody and the ILT4 antibodies.

The ILT4 antibodies increased production of the proinflammatory cytokines TNF-α and IL-6 in a dose-responsive manner, similarly to the control antibody. The results of the macrophage repolarization assay confirm that the ILT4 antibodies induce the repolarization of suppressive M2-type macrophages toward the proinflammatory M1 phenotype.

### Example 4

Potency of mouse anti-human Immunoglobulin-Like Transcript 4 (ILT4) antibodies for blockade of the interaction between human ILT4 and HLA-G: Mouse anti-human Immunoglobulin-Like Transcript 4 (ILT4) antibodies were tested for the ability to block the interaction between human ILT4 and HLA-G. A commercially available non-HLA-G blocking antibody was included as a negative control. Control antibodies were included as positive controls. Blocking activity was measured in a flow cytometry assay using soluble fluorescence-conjugated HLA-G and CHO-S/ILT4 cell lines engineered to express human ILT4 on the surface. Parental CHO-S cells that does not express human ILT4 were used as control.

The ability of control and candidate mouse anti-human ILT4 antibodies to block the interaction between human ILT4 and HLA-G was evaluated using soluble fluorescence-conjugated HLA-G and CHO-S/ILT4 cells. The cells, which express human ILT4, were preincubated with ILT4 antibodies then incubated with HLA-G. Flow cytometry was then used to quantify HLA-G binding to cells. The resultant cell-associated fluorescence was quantitated and used to calculate potency of ILT-4 blockade.

CHO-S and CHO-S/ILT4 Culture: CHO-S cells were cultured in CD OptiCHO medium and CHO-S/ILT4 cells were cultured in OptiCHO medium with 20 μg/ml puromycin in 5 percent carbon dioxide at 37 degrees C. with shaking at 150 revolutions per minute (RPM).

HLA-G Titration in HLA-G Binding Assay: CHO-S parental cells and CHO-S/ILT4 cells were pelleted by centrifugation and washed in FACS buffer (PBS+0.5% bovine serum albumin (BSA)) at 200,000 cells per ml. Cells were resuspended in Fluorescence-Activated Cell Sorting (FACS) buffer at 1,000,000 cells per ml, transferred into 96 well round bottom 96 well plate at 50,000 cells per well, and put on ice. All subsequent manipulations were performed on ice.

A primary tetramer dilution at 50 μg/ml was made in FACS buffer. In a separate 96 well plate, 1:2 serial dilutions of tetramer were prepared from 50 μg/ml to 0.05 μg/ml. From each dilution, 50 μl was added to cells in duplicate. Wells were mixed and incubated for 30 minutes at 4 degrees C. Cells were washed twice in FACS buffer and resuspended in 250 μl FACS buffer with 0.1 μg/ml propidium iodide.

Cells were acquired on an Attune flow cytometer, with 5000 cells collected per sample, and Mean Fluorescence Intensity (MFI) was recorded. MFI at different antibody concentrations was plotted with Prism software (Graphpad).

Antibody Titration in HLA-G Blocking Assay: CHO-S/ILT4 cells were pelleted by centrifugation and washed in FACS buffer (PBS+0.5% BSA) at 200,000 cells per ml. Cells were resuspended in FACS buffer at 1,000,000 cells per ml, transferred into a 96 well round bottom 96 well plate at 50,000 cells per well, and put on ice. All subsequent manipulations were performed on ice.

In the first blocking experiment, the negative control non-HLA-G blocking antibody 287219R (Lot: CMIF0221051 from R&D Systems, cat #MAB2078R) and the positive control HLA-G blocking control antibodies J19 h1 and 1E1 were evaluated. Primary antibody dilutions were made by diluting antibody to 30 μg/ml in FACS buffer. Ten threefold serial dilutions were then made in FACS buffer from 30 μg/ml to 0.001 μg/ml and 50 μl from each dilution was added to cells in duplicate. Control wells received FACS buffer with no antibody. Wells were mixed and incubated for 30 minutes at 4 degrees C. Cells were washed twice in FACS buffer.

In the second blocking experiment, the primary antibody dilutions were made by diluting antibody to 15 μg/ml in FACS buffer. Three independent sets of eleven 2.5-fold serial dilutions were then made in FACS buffer from 15 μg/ml to 0.002 μg/ml. Each dilution set was handled independently, and 50 μl from each was added to cells in duplicate. Control wells received FACS buffer with no antibody. Wells were mixed and incubated for 30 minutes at 4 degrees C. Cells were washed twice in FACS buffer.

In both blocking experiments, phycoerythrin (PE)-conjugated HLA-G tetramer was diluted in FACS buffer to 5 μg/ml and 100 μl per well was added to cells. Cells were incubated at 4 degrees C. for 30 minutes, washed twice in FACS buffer, and resuspended in 250 μl FACS buffer with 0.1 μg/ml propidium iodide.

Cells were acquired on an Attune flow cytometer, with 5000 propidium iodide-negative cells collected per sample, and MFI was recorded. Relative blocking activity was calculated with the following formula: 100−(Sample MFI−Background MFI)/(Top MFI−Background MFI)*100. Prism software (Graphpad) was used to plot MFI and relative blocking activity for each sample. The blocking potency for each antibody, represented as IC₅₀, was calculated with Prism software.

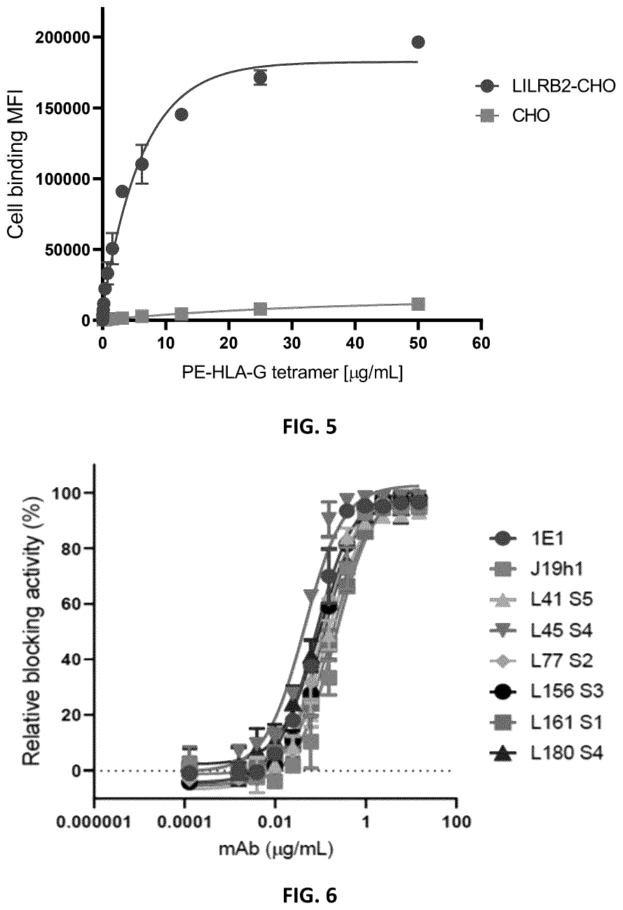

HLA-G Titration: Binding of PE-conjugated HLA-G to CHO-S parental cells and CHO-S/ILT4 cells was quantitated by flow cytometry in the PE channel. HLA-G bound CHO-S/ILT4 cells, with a maximum MFI that was 783-fold greater than untreated cells. Binding to parental CHO-S cells was lower, with a maximum MFI that was 43-fold greater than untreated cells (FIG. 5).

Based upon these plots, 5 μg/ml phycoerythrin-conjugated HLA-G was selected as the concentration at which to evaluate candidate antibodies for their ability to block HLA-G binding to CHO-S/ILT4 cells.

In the first experiment, preincubation with J19 h1 and 1E1 decreased PE-HLA-G MFI by up to 22- and 30-fold, respectively, from cells treated only with PE-HLA-G. Preincubation with the control antibody 287219R did not decrease the PE-HLA-G MFI IC₅₀ values for HLA-G blockade by J19 h1 and 1E1 were both 0.13 μg/ml. An IC₅₀ value could not be calculated for the control antibody 287219R (Table 1).

TABLE 1
Experiment 1 - IC₅₀ Values for Blockade of HLA-G
Binding by Control Anti-Human ILT4 Antibodies

| Antibody | IC50, μg/ml |
|---|---|
| 287219R | nd |
| J19h1 | 0.13 |
| 1E1 | 0.13 |

In the second experiment, the ILT4 antibodies blocked HLA-G binding to CHO-S/ILT4 cells as shown by decreased PE-HLA-G MFI on CHO-S/ILT4 cells. The results are shown as blocking activity EC₅₀ (μg/ml) in FIG. 6. The extent of the decrease depended upon the antibody, with L41S5 resulting in an up to 14-fold decrease and L45S4 resulting in an up to 69-fold decrease. IC₅₀ values for ILT4 antibodies ranged from 0.044 to 0.216 μg/ml (Table 2).

TABLE 2
Experiment 2 - IC₅₀ Values for Blockade of HLA-G Binding
by Candidate Mouse Anti-Human ILT4 Antibodies

| Antibody | IC₅₀, μg/ml |
|---|---|
| 1E1 | 0.075 |
| J19h1 | 0.216 |
| L41S5 | 0.134 |
| L45S4 | 0.044 |
| L77S2 | 0.103 |
| L156S3 | 0.113 |
| L161S1 | 0.182 |
| L180S4 | 0.095 |

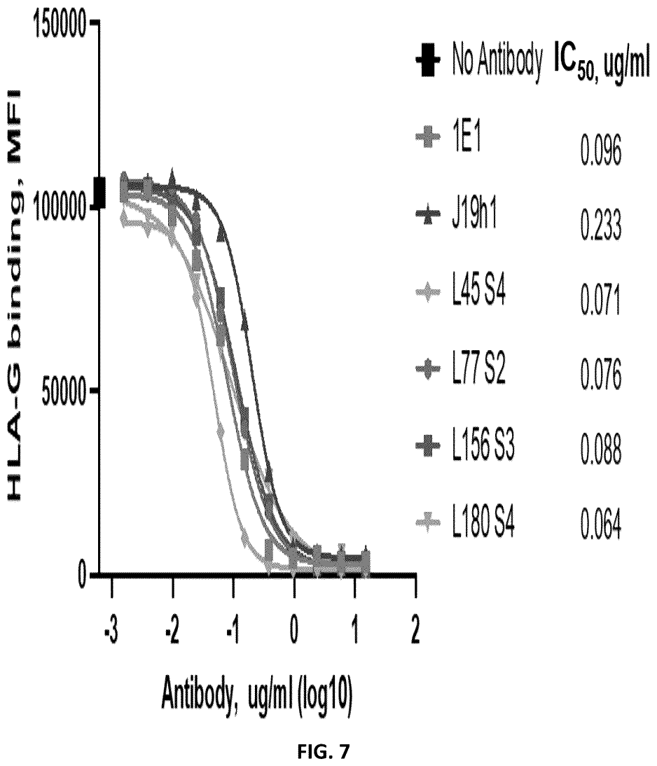

The results demonstrate the ability of mouse anti-human ILT4 antibodies to block the interaction between soluble HLA-G and cell-expressed ILT4 in a flow cytometry assay. The non-blocking antibody used as a negative control did not affect the HLA-G MFI on CHO-S/ILT4 cells, indicating there was no blockade of the HLA-G/ILT4 interaction. The two control antibodies used as positive controls caused a decrease in HLA-G MFI on CHO-S/ILT4 cells, demonstrating the assay detects blockade of the HLA-G/ILT4 interaction. The mouse anti-human ILT4 antibodies also blocked the HLA-G/ILT4 interaction. The effect on HLA-G MFI ranged from 14- to 69-fold decreases in MFI. Potency of the mouse anti-human ILT4 antibodies ranged from 0.065 to 0.171 μg/ml. FIG. 7 shows IC₅₀ curves from an exemplary ILT4 blocking of HLA-G assay using flow cytometry. Control antibodies, 1E1 and J19 h1, were included for comparison.

### Example 5

Surface plasmon resonance (SPR) analysis of mouse, chimeric, and humanized anti-ILT4 antibodies binding to ILTR4 (LILRB2): Mouse, chimeric, and humanized anti-ILT4 antibodies, as described above, were characterized for binding affinity to ILT4 (LILRB2) and blocking using SPR.

Label-free SPR analysis on a Biacore T200 instrument was utilized to measure antibody affinity to ILT4. CM4 sensor chips from Cytiva Inc. were utilized for all medium resolution analyses. Either anti-mouse Fc specific or anti-human Fc specific capture surfaces were prepared using standard amine coupling chemistry. Mouse, Chimera or Humanized mAbs were captured to their respective surfaces and ILT4 was injected across the surfaces from 101 nM to 0.79 nM in a 2-fold serial dilution series at a flow rate of 100 μL/min. The association phase was followed for 90 sec. while the dissociation phase was followed for 1200 sec. due to the medium resolution analysis implemented here. The sensorgrams were double-referenced and fit globally to a 1:1 interaction model with a term included for mass transport, and the equilibrium dissociation constant was calculated as K_{D}=k_{off}/kₒₙ. The slowest k_{off} that can be measured with a 5% decrease in the dissociation phase over 1200 seconds is 4.27×10⁻⁵ s⁻¹. Hence, if a k_{off} slower than 4.27×10⁻⁵ s⁻¹ was initially calculated from the global nonlinear fit of the data, the k_{off} was then held constant at 4.27×10⁻⁵ s⁻¹ during refitting in a subsequent global analysis. All kinetic experiments were performed at 25° C. in a buffer consisting of Hepes-buffered saline, pH 7.4, with 0.05% polysorbate 20 (HBS-P+) and 100 μg/ml Bovine Serum Albumin (BSA). Table 3 shows the results of all the binding analyses conducted.

TABLE 3

| mAb | kₒₙ × 10⁵ (M⁻¹s⁻¹) | k_{off} × 10⁻⁵ (s⁻¹) | K_{D} (pM) |
|---|---|---|---|
| Murine 45 | 4.01 | 8.4 | 210 |
| Chimeric 45 | 4.89 | 10.9 | 223 |
| Humanized 45_01 | 3.30 | 41.8 | 1270 |
| Murine 156 | 2.69 | 4.27 | 160 |
| Chimeric 156 | 3.73 | 4.27 | 114 |
| Humanized 156_03 | 4.97 | 4.27 | 86 |
| Murine 77 | 3.87 | 4.27 | 111 |
| Chimeric 77 | 7.28 | 4.27 | 59 |
| Humanized 77_01 | 8.35 | 4.27 | 52 |
| Murine 180 | 4.31 | 4.27 | 99 |
| Chimeric 180 | 8.74 | 4.27 | 49 |
| Humanized 180_04 | 8.12 | 4.27 | 53 |

Values emphasized with bold and underline signify that k_{off} was held constant at its theoretical limit of 4.27×10⁻⁵ s⁻¹ for 20 minutes of dissociation.

High resolution binding kinetics for ILT4 binding using humanized anti-ILT4-LALA antibodies were measured in triplicate and the results are summarized as follows (the numbers in parentheses are the 95% confidence intervals):

| | kₒₙ × 10⁶ | k_{off} × 10⁻⁵ | | K_{D} (pM) | | SEQ ID |
|---|---|---|---|---|---|---|
| mAb | (M⁻¹s⁻¹) | (s⁻¹) | | LALA | | NOs: |
| Humanized | 3.22 (0.20) | 1.83 | (0.35) | 5.7 | (1.4) | 128 and |
| 156_03 | | | | | | 129 |
| Humanized | 4.78 (0.47) | 4.24 | (0.41) | 8.9 | (1.5) | 126 and |
| 77_01 | | | | | | 127 |
| Humanized | 3.95 (1.03) | 0.63 | (0.30) | 1.6 | (1.1) | 130 and |
| 180_04 | | | | | | 131 |
| Humanized | 3.45 (0.39) | 53.1 | (0.28) | 154 | (9) | 124 and |
| 45_01 | | | | | | 125 |
| 1E1 | 1.62 (0.15) | 895 | (53) | 5500 | (200) | — |
| J19h1 | 5.11 (0.75) | 433 | (24) | 850 | (80) | — |

Anti-ILT4 antibodies Hz45 and Hz156 were assessed in a series of in vitro assays that evaluated 1) binding to recombinant human and Cynomolgus ILT4; 2) binding to ILT4 related molecules from LILRA and LILRB families; 3) the impact of binding to ILT4 on the inhibition of HLA-A and HLA-G binding to recombinant human ILT4.

Both antibodies bind ILT4 tightly with Hz45 having a K_{D}=154 PM and Hz156 a K_{D}=5.7 pM. The on-rates of both antibodies are similar at ˜3.3×10⁶ M⁻¹s⁻¹. Hz45 has a 29-fold faster off-rate, 53.1×10⁻⁵ s⁻¹, than Hz156 for ILT4, hence the weaker affinity for Hz45 mostly lies in the faster off-rate observed as compared to that of Hz156.

Both antibody molecules showed no binding to recombinant Cynomolgus monkey ILT4 nor to recombinant human LILRA1-6 and LILRB1-4 family members except for LILRB2 (ILT4). Antibodies Hz45 or Hz156 pre-complexed with recombinant human ILT4 blocks ILT4 binding to HLA-A and HLA-G.

All experiments described herein were performed using a Biacore T200 instrument at 25° C. with a running buffer of Hepes-Buffered Saline, 0.05% polysorbate 20, pH 7.4 (HBS-P+) with 100 μg/mL Bovine Serum Albumin (BSA) added. The running buffer used for immobilizing antibody to the surface was HBS-P+ with no BSA added.

Surface plasmon resonance spectroscopy was used to assess binding between ILT4 and antibodies Hz45 and Hz156. The binding affinity of each antibody for ILT4 was measured and both interactions exhibited a fast association rate constant, but Hz156 had a 29-fold slower dissociation rate constant than Hz45. Both antibodies Hz45 and Hz156 exhibited strong affinities for ILT4 of 154 pM and 5.7 pM, respectively. Both molecules showed no binding to recombinant Cynomolgus monkey ILT4 nor to recombinant human LILRA1-6 and LILRB1-4 family members except for LILRB2 (ILT4). Hz45 or Hz156 pre-complexed with recombinant human ILT4 blocks ILT4 binding to HLA-A and HLA-G.

### Kinetic Exclusion Assay

Kinetic Exclusion Assay (KinExA®) was performed to measure the equilibrium binding affinity and kinetics between human ILT4 and four humanized LALA-Fc modified antibodies in solution. For affinity analysis, the equilibrium dissociation constant, K_{D}, was experimentally determined and reflects the strength of the binding interaction. The rate of association, kₒₙ, was also experimentally determined, while the rate of dissociation, k_{off}, is calculated based on the following equation: k_{off}=K_{D}×kₒₙ

A K_{D} analysis requires immobilization of one interaction partner (the titrated binding partner) to a solid phase which is then used as a probe to capture the other interaction partner, the constant binding partner (CBP). For each experiment, one of the binding partners is titrated in a background of the CBP and allowed to reach equilibrium. The solutions are then briefly exposed to the solid phase and a portion of free CBP is captured. The captured CBP is then labeled with a fluorescent secondary molecule. The short contact time with the solid phase is less than the time needed for dissociation of the pre-formed complex in solution, thus competition between the solution and the solid phase titrated binding partner is “kinetically excluded.” Since the solid phase is only used as a probe for the free CBP in each sample, the solution equilibrium is not altered during KinExA measurements.

The signals generated from the captured CBP, which are directly proportional to the concentration of free CBP in the equilibrated samples, were used to determine the K_{D} value. The KinExA Pro software performs a least squares analysis on the measured data to fit an optimal solution for the K_{D} of a 1:1 reversible bi-molecular interaction.

Determination of binding kinetics is completed in a similar format as the equilibrium analysis, except measurements are collected “pre-equilibrium” and the binding signals are a function of time. The kinetics experiment is referred to as the direct method. This method holds the titrated and CBP concentrations constant and the solution is probed over time. The amount of the free CBP in the solution will decrease as the sample moves toward equilibrium.

The binding affinity (K_{D}), association (kₒₙ), and dissociation rate constants (k_{off}) were measured for 4 different Humanized LALA IgG antibodies to ILT4 using KinExA. An n-curve analysis of at least two different curves, run at two different CBP concentrations, was used to determine the K_{D} of the interactions The ILT4 was used as the concentration reference for all curves. All of the binding systems have low error and well defined 95% confidence intervals giving confidence to the measurements:

| | Hu IgG | Hu IgG | Hu IgG | Hu IgG |
|---|---|---|---|---|
| | 45_01 | 180_04 | 156_03 | 77_01 |
| | LALA | LALA | LALA | LALA |
| K_{D} | 85.1 pM | 759 fM | 9.16 pM | 5.46 pM |
| 95% Confidence | 67.4 pM- | 470 fM- | 5.51 pM- | 4.22 pM- |
| Interval | 106 pM | 1.11 pM | 13.9 pM | 6.91 pM |

Competitive binding studies of ILT4 between HLA-A and HLA-G and anti-ILT4 antibodies as probed by SPR: SPR spectroscopy on a Biacore T200 instrument was used to probe whether four chimeric and humanized anti-ILT4 mAbs could block LILRB2 from binding to HLA-A (A*02:01; Immunitrack, Denmark) and HLA-G (monomeric HLA-G was prepared at the Fred Hutchinson Cancer Center in Seattle, WA). Neutravidin was immobilized to all four flows of a CM4 chip using standard amine coupling chemistry, and biotinylated HLA-G was captured on flow cell 2 and biotinylated HLA-A was captured on flow cell 4 with both flow cells 1 and 3 serving as references, respectively. A “pre-mix” format was used to perform these competitive experiments wherein 514 nM ILT4 was mixed with ˜600 nM mAb binding site. At these concentrations, ˜0.1% of the 514 nM ILT4 in solution would be free at equilibrium for most of the antibodies studied. The ILT4/mAb complex was injected for 60 sec. across all four sensor surfaces at 30 μL/min in HBS-P+ buffer containing 100 μg/mL BSA. The dissociation phase was followed for 60 seconds and all sensorgrams were double-referenced. If a binding signal for any given ILT4/mAb complex was observed that mAb was designated a non-competitive binder for ILT4 binding. Conversely, if no significant binding signal was observed for binding of a particular ILT4/mAb complex to captured HLA-A or HLA-G, then that mAb was designated a competitive binder for ILT4. All four chimeric and humanized mAbs investigated were competitive binders of ILT4 with HLA-A and HLA-G.

SPR and flow based characterization of blocking activity for humanized (Hz) versions of L45S4, L77S2, L156S3, and L180S4 between ILT4 and HLA-G. Table 4, below, shows the characterization of the humanized ILT4 antibodies (the numbers in parentheses are the 95% confidence intervals).

TABLE 4

| | Hz 180 04 | Hz 156 03 | Hz 45 01 | Hz 77 01 |
|---|---|---|---|---|
| Affinity (K_{D} | 1.6 (1.1) | 5.7 (1.4) | 154 (9) | 8.9 (1.5) |
| (pM)) | | | | |
| IC₅₀ in HLA- | 0.051 | 0.212 | 0.092 | 0.176 |
| G blocking | | | | |
| (μg/ml) | | | | |

SPR Cross-reactivity study of Four Humanized anti-ILT4 antibodies with nine LILRA and LILRb homologues: A number of LILRA and LILRB family members share a high amount of homology with LILRB2 (ILT4) as shown in Table 5. Hence, determining the specificity and cross-reactivity profiles of the humanized anti-ILT4 antibodies to other family members becomes of paramount importance in characterizing the binding and functional properties of the humanized anti-ILT4 mAbs.

SPR was used to determine the specificity of four humanized anti-ILT4 mAbs for binding to LILRB2 and to determine any cross-reactivity with nine other LILRA and LILRB family members. LILR proteins were purchased from Acro Biosystems or R&D Systems, with sequence provided below. The studies were performed with a Biacore 8K instrument using a CM4 sensor chip with a running buffer of HBS-P+, 100 μg/mL BSA. Anti-human Fc specific capture surfaces were prepared using standard amine coupling chemistry. MAb was captured to flow cell 2 with flow cell 1 serving as the reference flow cell. Each of the nine family members and LILRB2 were injected at 100 nM at 30 μL/min for 90 sec with 240 sec of dissociation. Cross-reactivity was determined by examination of the sensorgrams that possessed a significant binding signal in addition to displaying a shape for a true molecular binding interaction. All sensorgrams were doubled-referenced during processing of the data. Table 5 shows the results from the cross-reactivity study. MAbs 45_01, 77-01, and 156_03 show no cross-reactivity to any family members and only bind to LILRB2. MAb 180_04 shows some cross-reactivity to family members LILRA1, LILRA3, and LILRB1. The results indicate that greater than 79% homology with LILRB2 was required for cross-reactivity with only mAb 180_04. The strongest cross-reactive binding interaction observed with mAb 180-04 was with LILRA3 and appears to have a K_{D} in the range of 600-700 nM.

The methods described above were performed on the Biacore T200 to determine if any of these four mAbs cross-reacted with Cynomolgus LILRB2. No cross-reactivity was observed for any of these mAbs (Table 5).

TABLE 5

| | | | | | | | | | | | Cyno |
|---|---|---|---|---|---|---|---|---|---|---|---|
| | | | | | | | | | | | LILRB2 |
| mAb | LILRA1 | LILRA2 | LILRA3 | LILRA4 | LILRA5 | LILRA6 | LILRB1 | LILRB2 | LILRB3 | LILRB4 | (Putative) |
| 45_01 | − | − | − | − | − | − | − | +++ | − | − | − |
| 77_01 | − | − | − | − | − | − | − | ++++ | − | − | − |
| 156_03 | − | − | − | − | − | − | − | ++++ | − | − | − |
| 180_04 | + | − | + | − | − | − | +/− | ++++ | − | − | − |
| % Homology | 85 | 79 | 81 | 66 | 54 | 69 | 79 | 100 | 70 | 49 | 80 |
| of ECD | | | | | | | | | | | |
| relative | | | | | | | | | | | |
| to LILRB2 | | | | | | | | | | | |

− no binding
+/− extremely weak binding (~mM K_{D})
+ weak binding (~600-700 nM K_{D})
+++ binding (1-2 nM K_{D})
++++ tight binding (<100 pM K_{D})

### Example 6-Blocking Activities of Humanized Anti-ILT4 Antibodies

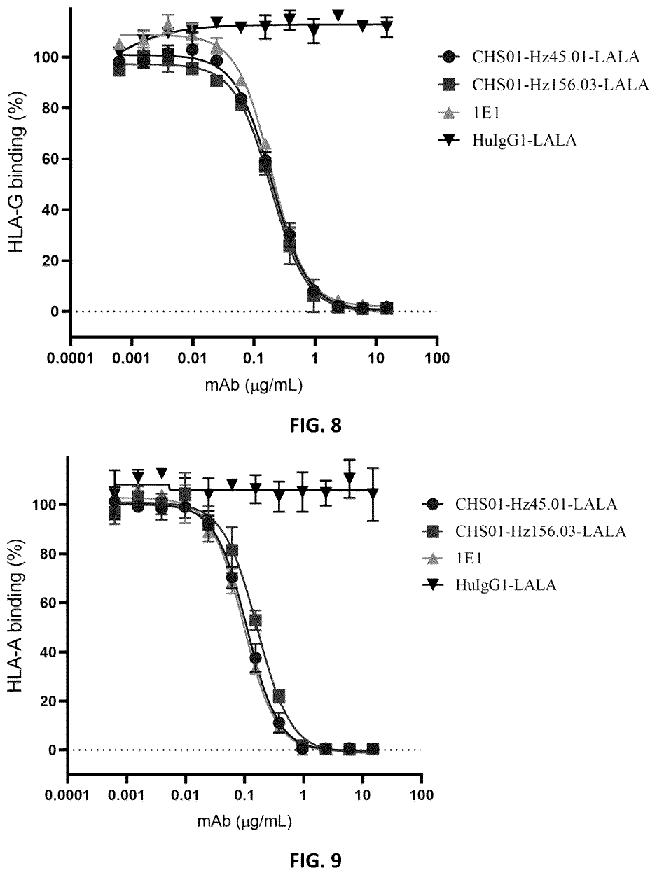

To demonstrate the anti-ILT4 ability to block ILT4 binding to its ligands: HLA-A and HLA-G, the humanized antibodies Hz45.01-LALA, Hz156.03-LALA, and 1E1, and human IgG1-LALA control were tested with serial titrations (0-15 μg/mL) in triplicates in functional blocking assay blocking interaction between R-phycoerythrin (PE)-HLA-G tetramer and ILT4-CHO cell (FIG. 8), or PE-HLA-A tetramer at 5 ug/ml (FIG. 9). The data are summarized in Tables 6 and 7:

TABLE 6
IC₅₀ value of anti-ILT4 blocking HLA-G binding to ILT4

| | CHS01- | CHS01- | |
|---|---|---|---|
| IC₅₀ (ug/ml) | Hz45.01-LALA | Hz156.03-LALA | 1E1 |
| Experiment 1 | 0.1982 | 0.1904 | 0.1937 |
| Experiment 2 | 0.1195 | 0.1397 | 0.162 |
| Experiment 3 | 0.1846 | 0.2326 | 0.151 |

TABLE 7
IC₅₀ value of anti-ILT4 blocking HLA-G binding to ILT4

| | CHS01- | CHS01- | |
|---|---|---|---|
| IC₅₀ (ug/ml) | Hz45.01-LALA | Hz156.03-LALA | 1E1 |
| Experiment 1 | 0.1058 | 0.1493 | 0.1016 |
| Experiment 2 | 0.1221 | 0.1516 | 0.1071 |
| Experiment 3 | 0.1092 | 0.1635 | 0.09561 |

Anti-ILT4 antibodies blocks both HLA-G and HLA-A efficiently with similar IC₅₀ range.

### Example 7: Macrophage Polarization Study

The ability of the anti-ILT4 constructs (final format, humanized Ab with LALA mutation on the Fc receptor) to polarize M2 macrophages to M1 macrophages to produce proinflammatory cytokines TNF-α and IL-6 on in vitro differentiated macrophages was evaluated.

### Human Monocyte Isolation and Macrophage Differentiation:

PBMCs were isolated from 4 healthy human donors by Ficoll-Paque gradient centrifugation. Untouched human monocytes were then purified from PBMCs using the Dynabeads Untouched Human Mononocyte Kit per manufacturer's instructions (ThermoFisher Scientific). Monocytes were cultured in RPMI with 10% FBS and 50 ng/ML M-CSF for 7 days to generate macrophages.

### Treatment of Macrophages and Determination of Cytokine Production

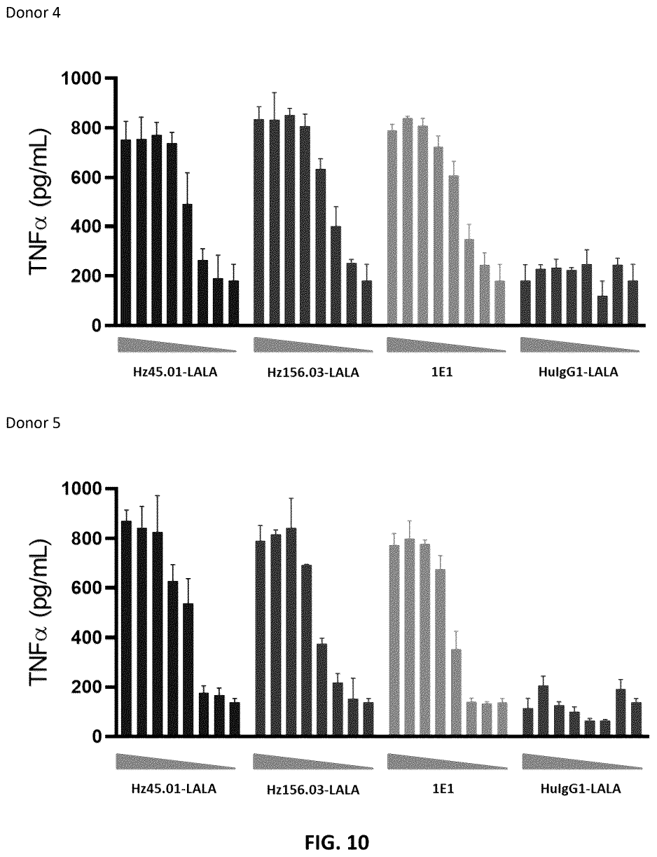

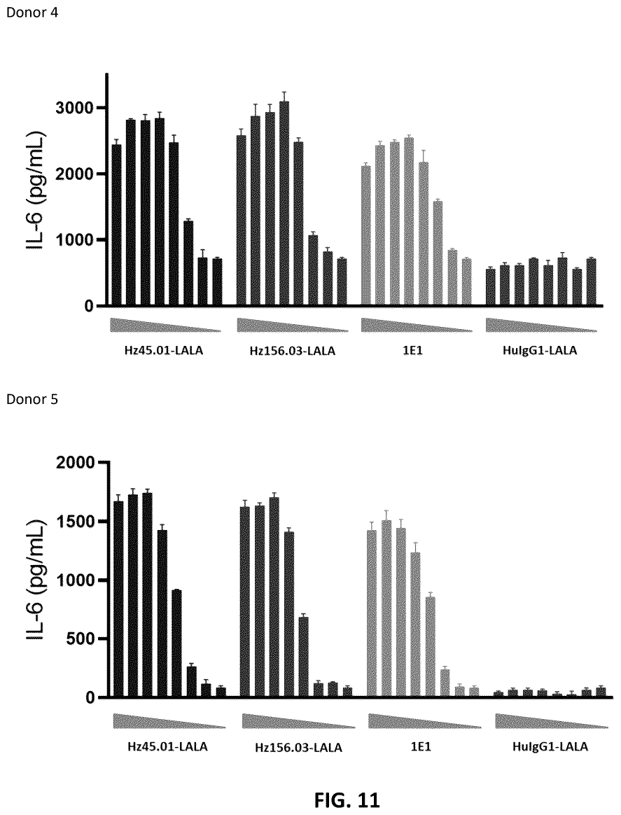

Macrophages were harvested and plated at 1×10⁵ cells per well in a 96-well plate. Macrophages were incubated with humanized LALA anti-ILT4 antibodies or human IgG1-LALA control in a serial titration from 4 μg/ml with 1:4 dilution in the presence of 10 ng/ml of LPS for 24 h. All samples were tested in triplicate. Conditioned media were then collected for analysis of IL-6 and TNFa production using the ELISA assay kits per manufacturer's instructions (R&D Systems) as shown in FIGS. 10 and 11. The ELISA data were analyzed and the EC₅₀ values were determined using the nonlinear regression formula with the Prism GraphPad software as shown in Tables 8 and 9.

TABLE 8
EC₅₀ value of TNFa production and R square value

| EC₅₀ (ng/mL) | Hz45.01 | Hz156.03 | 1E1 |
|---|---|---|---|
| donor 4 | 13.75 | 7.24 | 8.77 |
| donor 5 | 18.48 | 23.57 | 23.49 |
| R² | Hz45.01 | Hz77.01 | Hz156.03 |
| donor 4 | 0.9479 | 0.9639 | 0.9736 |
| donor 5 | 0.9375 | 0.9687 | 0.9837 |

TABLE 9
EC₅₀ value of IL-6 production and R square value

| EC₅₀ (ng/mL) | Hz45.01 | Hz156.03 | 1E1 |
|---|---|---|---|
| donor 4 | 6.03 | 8.20 | 3.74 |
| donor 5 | 15.92 | 21.3 | 13.92 |
| R² | Hz45.01 | Hz77.01 | Hz156.03 |
| donor 4 | 0.9775 | 0.9719 | 0.9609 |
| donor 5 | 0.9949 | 0.9959 | 0.9901 |

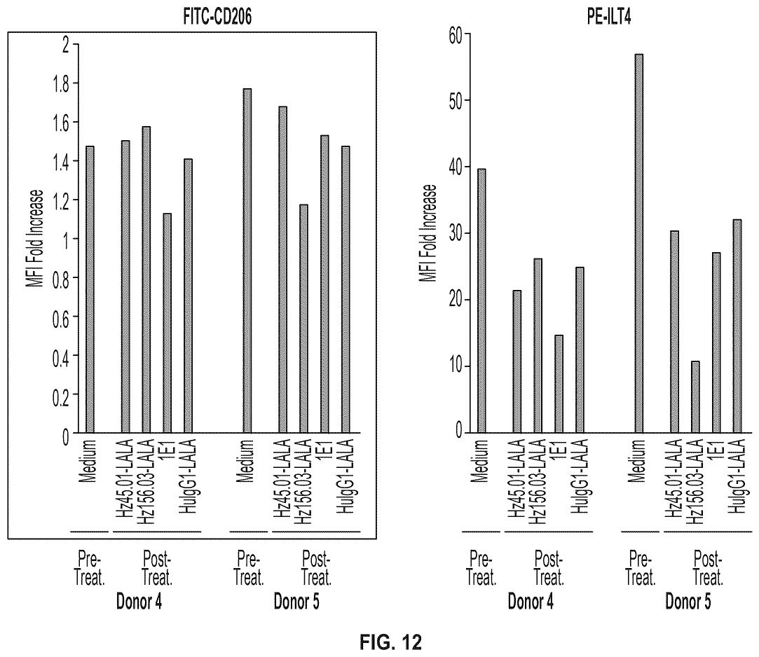

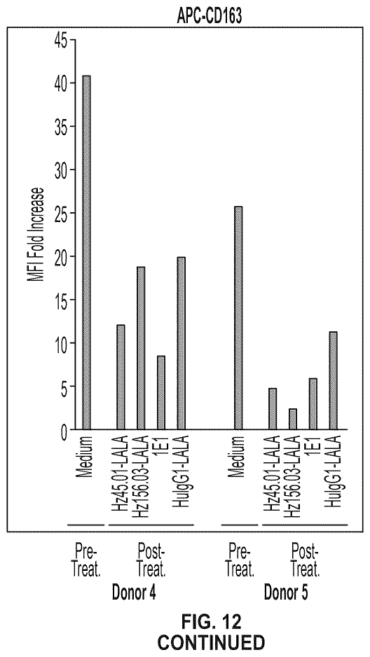

Cell surface markers (ILT4, CD163, CD206) on macrophages before and after treatment were analyzed by flow cytometry as shown in FIG. 12.

Macrophages from two different donors treated with anti-ILT4 antibodies produced TNF-α and IL-6 proinflammatory cytokine with EC₅₀ values in the range of ng/ml. Macrophages before and after antibody treatment expressed ILT4. Although anti-ILT4 antibodies had minimal effect on reducing CD206 expression, CD163 surface expression was decreased on macrophages from both donors. CD163 is considered a M2 marker. Together, these data show that anti-ILT4 antibody treatment reprogrammed M2 to M1 macrophages by reducing CD163 expression and induce TNF-α and IL-6 proinflammatory cytokine production.

### Example 8: Macrophage Mixed Leuokcyte Reaction (MLR) Assay

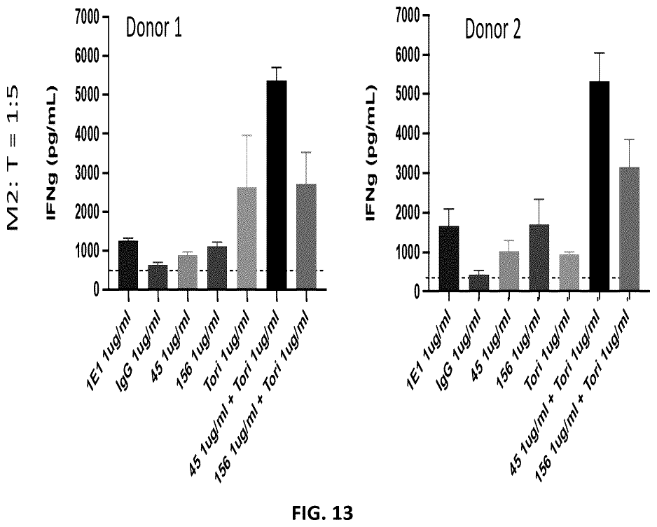

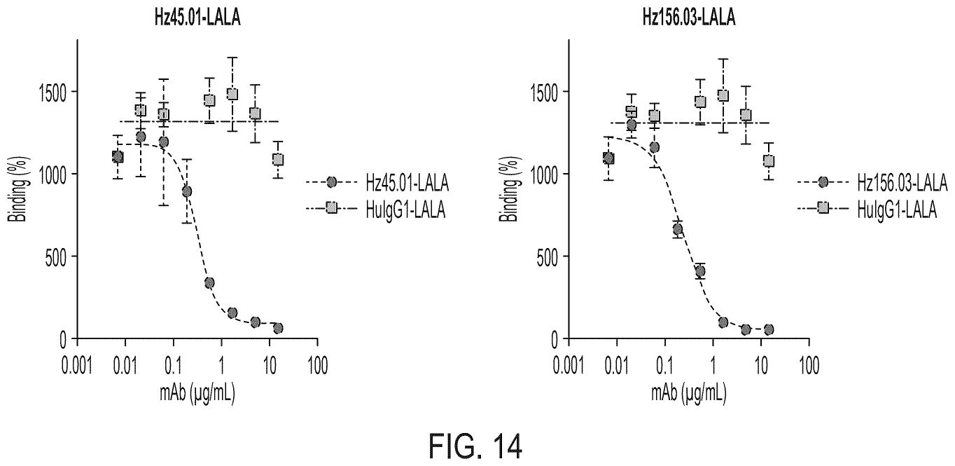

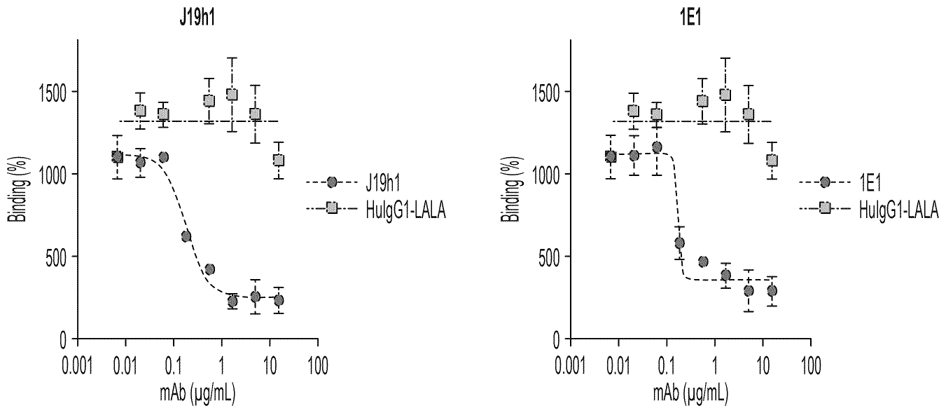

To assess the effect of humanized LALA anti-ILT4 antibody treatment on macrophage to enhance CD4 T cell activation as mono or combination treatment with anti-PD1 antibody as shown in FIG. 13.

### Human Monocyte Isolation and Macrophage Differentiation:

PBMCs were isolated from healthy human donors by Ficoll-Paque gradient centrifugation. Human monocytes were then purified from PBMCs using EasySep Human Monocyte isolation kit (StemCell) per manufacturer's instructions.

### Human Monocyte Differentiation and Culture Assay

The cell density was adjusted to 0.5×10e6 cells/ml in fresh differentiation medium (STEMCELL) and plate 10 ml/dish of monocytes into five 10 cm dishes+50 ng/ml M-CSF (add 5 ul of stemcell 100 μg/ml human M-CSF to 10 ml ImmunoCult™-SF Macrophage Medium) per donor. After 4 days, half additional differentiation medium was added (5 ml medium for each dish). After 2 additional days, cells were detached using 5 ml Versene Solution to each dish, spun down and the cells were resuspended in RPMI+10% FBS+1% P/S.

The cells were counted and prepared in RPMI+10% FBS+1% P/S medium at the density of 0.5×10e6 cells/ml. 100 ul of the cell suspension was added to the designed wells of two 96-well U-bottom plates following the plate map. Anti-ILT4 antibodies (humanized LALA) were added at a final concentration of 1 ug/ml to the designed wells (96-well plate).

CD4+ T cells were isolated from a different donor and prepare the CD4+ T cells at the density of 5×10e6 cells/ml and add the cell suspension to the designed wells. The plates were incubated at 37° C. 5% CO₂ for 5 days. At day 6, pipette up and down and transfer all solution to V-bottom plate. Spin down and collect the supernatant 100 ul from the 96-well plate for IFN-g cytokine analysis.

Using INF-g as a readout for T cell activation, observed IFN-g production as single agent when compared to isotype control. Observed increased IFN-g production when combined with anti-PD1 (Toripalimab) as shown in FIG. 13.

### Example 9: Completion of Anti-ILT4 mAbs Binding to ILT4-CHO

Competition binding assays were performed. The assays involved testing competition of Hz54.01-LALA (SEQ ID NO: 124 (HC), 125 (LC)), Hz156.03-LALA (SEQ ID NO: 128 (HC), 129 (LC)), 1E1, J19 h1, R&D Systems MAB2078R, and human IgG1-LALA with serial titrations (0-15 μg/mL) in a competition binding assays with Alexa Fluor 647 (AF647)-Hz45.01-LALA or AF647-Hz156.03-LALA for binding to ILT4-CHO cells. Binding activities were measured by flow cytometry.

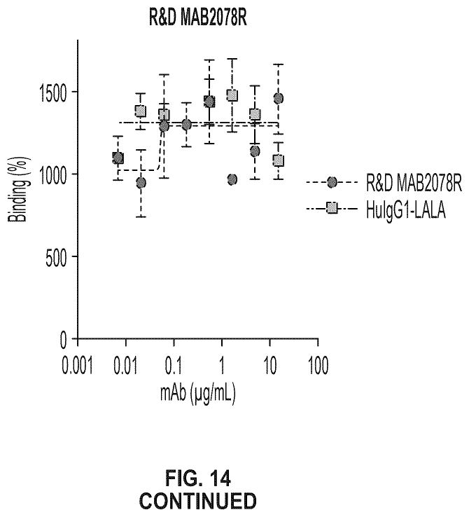

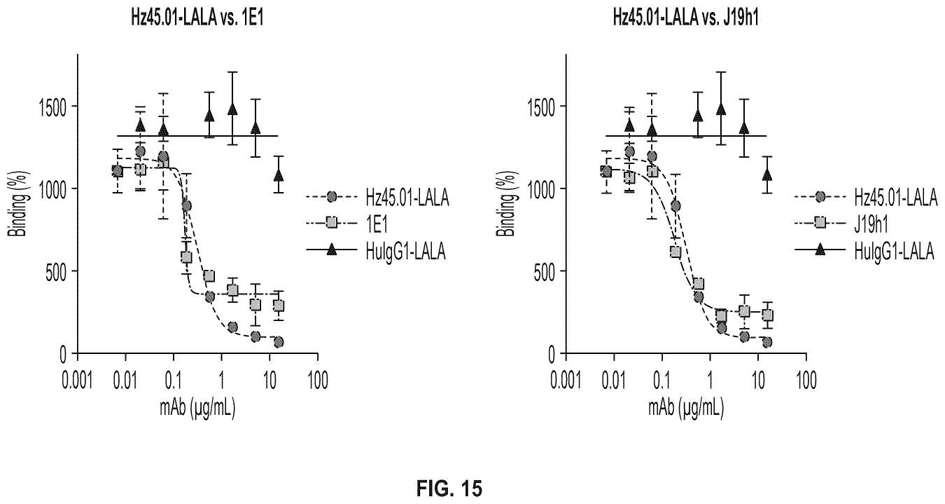

Anti-human ILT4 mAbs at various concentrations (0-15 μg/ml) were incubated with ILT4-CHO cells. Cells were washed and stained with AF647-Hz45.01-LALA. Cells were analyzed by flow cytometry. FIG. 14 shows competition of Anti-ILT4 mAbs binding to ILT4-CHO cells in competition with AF647-Hz45.01-LALA. FIG. 15 shows the competition binding of Hz45.01-LALA vs. 1E1 and HuIgG1-LALA (as a control) and Hz45.01-LALA vs. J19 h1 and HuIgG1-LALA (as a control).

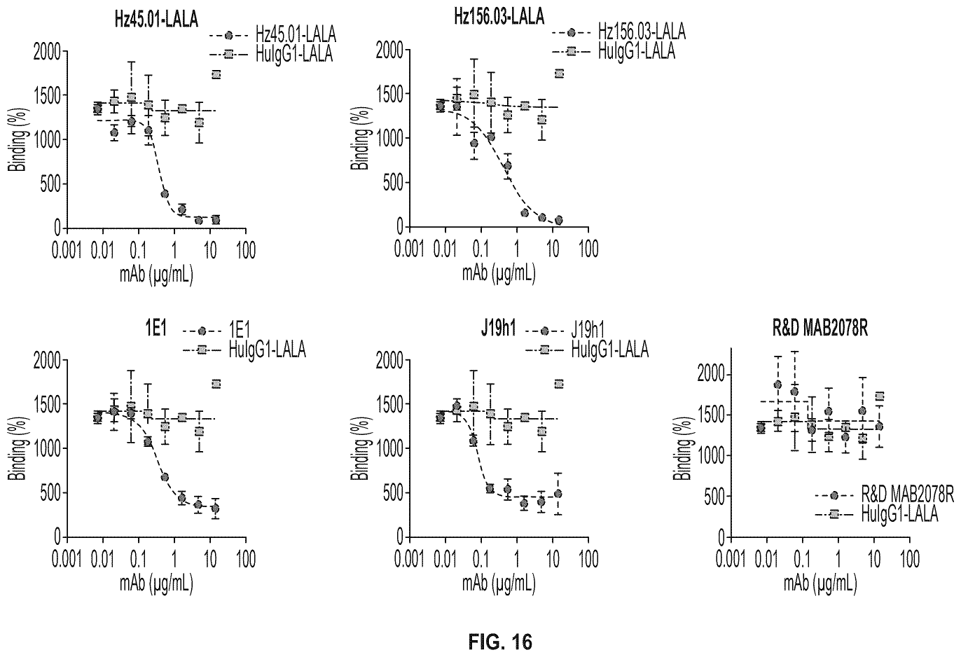

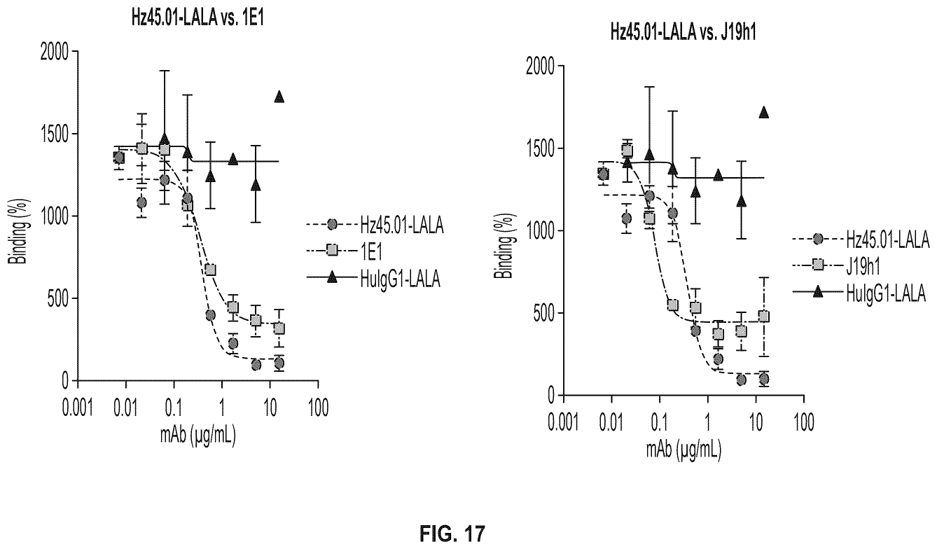

Anti-human ILT4 mAbs at various concentrations (0-15 μg/ml) were incubated with ILT4-CHO cells. Cells were washed and stained with AF647-Hz156.03-LALA. Cells were analyzed by flow cytometry. FIG. 16 shows competition of Anti-ILT4 mAbs binding to ILT4-CHO cells in competition with AF647-Hz156.03-LALA. FIG. 17 shows the competition binding of Hz45.01-LALA vs. 1E1 and HuIgG1-LALA (as a control) and Hz45.01-LALA vs. J19 h1 and HuIgG1-LALA (as a control).

All tested antibodies showed dose-dependent blocking of binding to ILT4-CHO cells and IC₅₀ values are summarized in the following table:

| | Hz45.01- | Hz156.03- | | |
|---|---|---|---|---|
| IC₅₀ (μg/mL) | LALA | LALA | 1E1 | J19h1 |
| Competition for Hz45.01- | 0.3097 | 0.2454 | 0.1705 | 0.182 |
| LALA | | | | |
| Competition for Hz156.03- | 0.3809 | 0.4219 | 0.3362 | 0.0796 |
| LALA | | | | |

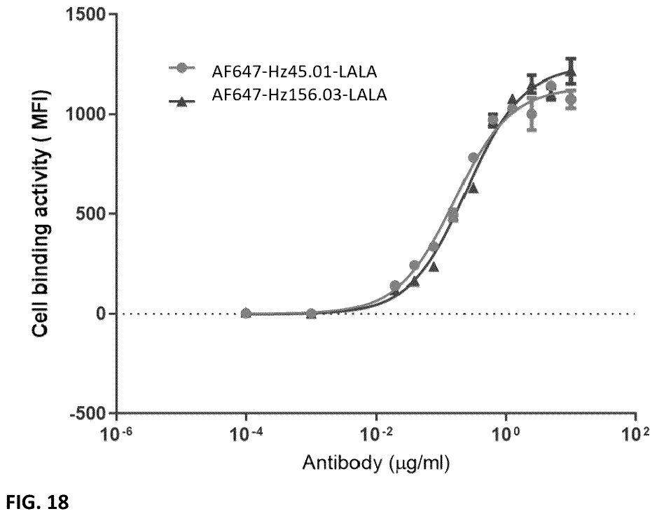

The binding of AF647-Anti-ILT4 mAbs to ILT4-CHO cells was measured as shown in FIG. 18. AF647-Hz45.01-LALA and AF647-Hz156.03-LALA at various concentrations (0-10 μg/ml) were incubated with ILT4-CHO cells and analyzed by flow cytometry. The tested antibodies showed dose-dependent binding to ILT4-CHO cells and EC₅₀ and EC₉₀ values are summarized in the following table:

| | Hz45.01-LALA | Hz156.03-LALA |
|---|---|---|
| EC₅₀ (mg/mL) | 0.1619 | 0.2477 |
| EC₉₀ (mg/mL) | 1.106 | 1.489 |

Hz45.01-LALA, Hz156.03-LALA, 1E1, and J19 h1 competed with binding of AF647-Hz45.01 to ILT4-CHO cells;
Hz45.01-LALA, Hz156.03-LALA, 1E1, and J19 h1 also competed with binding of AF647-Hz156.03-LALA to ILT4-CHO cells;
R&D MAB2078R and HuIgG1-LALA did not compete with binding of AF647-Hz45.01-LALA or AF647-Hz156.03-LALA to ILT4-CHO cells;
Hz45.01-LALA and Hz156.03-LALA completely competed binding of AF647-Hz45.01-LALA and AF647-Hz156.03-LALA;
Some levels of AF647-Hz45.01-LALA and AF647-Hz156.03-LALA binding activities remained with high doses of 1E1 and J19 h1 competition. The data indicate that Hz45.01-LALA and Hz156.03-LALA have more similar binding properties to each other and somewhat different binding properties compared to 1E1 and J19 h1.

| Description | SEQ ID NO | Sequence |
|---|---|---|
| L45S4 Heavy | SEQ ID NO: 1 | DYYMN |
| Chain CDR | SEQ ID NO: 2 | DINPNNGGTSYNQKFKG |
| | SEQ ID NO: 3 | GGAELTGTYWYFDV |
| L45S4 Light | SEQ ID NO: 4 | RASENIYSNLA |
| Chain CDR | SEQ ID NO: 5 | GATNLAD |
| | SEQ ID NO: 6 | QHFWDSPFT |
| L45S4 Heavy | SEQ ID NO: 7 | EVQLQQSGPELVKPGASVKISCKASGYTFTDY |
| Chain Variable | | YMNWVKQSHGKSLEWIGDINPNNGGTSYNQK |
| Region | | FKGKATLTVDKSSSTAYMELRSLTSEDSAVYY |
| (mIgG2a) | | CARGGAELTGTYWYFDVWGTGTTVTVSS |
| L45S4 Light | SEQ ID NO: 8 | DIQMTQSPASLSISVGETVTITCRASENIYSNLA |
| Chain Variable | | WYQQKQGKSPQVLVYGATNLADGVPSRFSGS |
| Region (kappa) | | GSGTQYSLKIKSLQSEDFGSYYCQHFWDSPFT |
| | | FGSGTKLEIK |
| L77S2 Heavy | SEQ ID NO: 9 | GYSVN |
| Chain CDR | SEQ ID NO: 10 | RINPYNGDIFNNQKFKG |
| | SEQ ID NO: 11 | GTTVGGAWFAY |
| L77S2 Light | SEQ ID NO: 12 | RASESVDSYGYSFLH |
| Chain CDR | SEQ ID NO: 13 | LASNLES |
| | SEQ ID NO: 14 | QQSNEDLMYT |
| L77S2 Heavy | SEQ ID NO: 15 | DVQLQQSGPELVKPGNSVKISCKAAGYSFTGY |
| Chain Variable | | SVNWVKERHGKSLEWIGRINPYNGDIFNNQKF |
| Region | | KGKATLTVDKSSSTAHMELRSLTSEDSAVYYC |
| (mIgG1) | | ARGTTVGGAWFAYWGQGTLVTVSA |
| L77S2 Light | SEQ ID NO: 16 | VIVLTQSPASLAVSLGQRAAISCRASESVDSYG |
| Chain Variable | | YSFLHWYQQKPGQPPKLLIYLASNLESGIPARF |
| Region (kappa) | | SGSGSGTDFTLTINPVEADDVATYYCQQSNED |
| | | LMYTFGGGTKLEIK |
| L156S3 Heavy | SEQ ID NO: 1 | DYYMN |
| Chain CDR | SEQ ID NO: 17 | RIYPGVYRTHYNEKFKD |
| | SEQ ID NO: 18 | SGYYGGTYEEDAMDY |
| L156S3 Light | SEQ ID NO: 4 | RASENIYSNLA |
| Chain CDR | SEQ ID NO: 19 | AATSLAD |
| | SEQ ID NO: 20 | QNFWDTPYT |
| L156S3 Heavy | SEQ ID NO: 21 | QVQLKQSGAELVRPGASVKLSCRASGYTFTD |
| Chain Variable | | YYMNWVKQRPGQGLEWIARIYPGVYRTHYN |
| Region | | EKFKDKATLTAEKSSSTAYMELSSLTSEDSAV |
| (mIgG2a) | | YFCARSGYYGGTYEEDAMDYWGQGTSVTVS |
| | | S |
| L156S3 Light | SEQ ID NO: 22 | DIQMTQSPASLSVSVGETVTITCRASENIYSNL |
| Chain Variable | | AWYQQKQGKSPQLLVYAATSLADGVPSRFRG |
| Region (kappa) | | SGSGTQYSLKISSLQSEDFGNYYCQNFWDTPY |
| | | TFGGGTKLEIK |
| L161S1 Heavy | SEQ ID NO: 23 | DSYMN |
| Chain CDR | SEQ ID NO: 24 | YINPDNGVTRYNQKFKG |
| | SEQ ID NO: 25 | EGTITTDLSWFAY |
| L161S1 Light | SEQ ID NO: 4 | RASENIYSNLA |
| Chain CDR | SEQ ID NO: 26 | ASTNLAD |
| | SEQ ID NO: 27 | QHFWDTPYT |
| L161S1 Heavy | SEQ ID NO: 28 | EVQLQQSGPELVIPGASVKISCKASGYTFTDSY |
| Chain Variable | | MNWVKQSHGKSLEWIAYINPDNGVTRYNQKF |
| Region | | KGKATLTVHKSSSTAYMELRSLTSEDSAVYYC |
| (mIgG2a) | | AREGTITTDLSWFAYWGQGTLVTVSA |
| L161S1 Light | SEQ ID NO: 29 | DIQMTQSPASLSVSVGETVTITCRASENIYSNL |
| Chain Variable | | AWYQQKQGKSPQLLVYASTNLADGAPATFSG |
| Region (kappa) | | SGSGTQYSLKINSLQSVDFGSYYCQHFWDTPY |
| | | TFGGGTKLEIK |
| L180S4 Heavy | SEQ ID NO: 30 | GYFMN |
| Chain CDR | SEQ ID NO: 31 | RINPYNGDIFYNQKFKG |
| | SEQ ID NO: 32 | GITVAAGSFDV |
| L180S4 Light | SEQ ID NO: 33 | RASESVDNYGNTFMH |
| Chain CDR | SEQ ID NO: 34 | RASNLES |
| | SEQ ID NO: 35 | QQSSDHPLT |
| L180S4 Heavy | SEQ ID NO: 36 | EVHLQQSGPELVKPGASVKISCKASGYSFIGYF |
| Chain Variable | | MNWMKQSHGKSLEWIGRINPYNGDIFYNQKF |
| Region | | KGKATLTVDKSSTTAHMDLLSLTSEDFAVYY |
| (mIgG1) | | CARGITVAAGSFDVWGTGTTVTVSS |
| L180S4 Light | SEQ ID NO: 37 | DIVLTQSPASLAVSLGQRATISCRASESVDNYG |
| Chain Variable | | NTFMHWYQQKPGQPPKLLIYRASNLESGIPAR |
| Region (kappa) | | FSGSGSKTDFTLTINPVEADDVATYYCQQSSD |
| | | HPLTFGAGTKLELS |
| L41S5 Heavy | SEQ ID NO: 38 | SYWMN |
| Chain CDR | SEQ ID NO: 39 | QIYPGHGDTNYNGKFKG |
| | SEQ ID NO: 40 | EGSELGRLFAY |
| L41S5 Light | SEQ ID NO: 41 | SASSSVSFMY |
| Chain CDR | SEQ ID NO: 42 | LTSNLAS |
| | SEQ ID NO: 43 | QQWSSNPPT |
| L41S5 Heavy | SEQ ID NO: 44 | QVQLQQSGAELVKPGASVKISCKASGYAFSSY |
| Chain Variable | | WMNWVKQRPGKGLEWIGQIYPGHGDTNYNG |
| Region | | KFKGKATLTADKSSSTAYMQLSSLTSEDSAVY |
| (mIgG2a) | | FCAKEGSELGRLFAYWGQGTLVTVSA |
| L41S5 Light | SEQ ID NO: 45 | QIVLTQSPALMSASPGEKVTMTCSASSSVSFM |
| Chain Variable | | YWYQQKPRSSPKPWIYLTSNLASGVPARFSGS |
| Region (kappa) | | GSGTSYSLTISSMEAEDAATYYCQQWSSNPPT |
| | | FGGGTKLEIK |
| L105S6 Heavy | SEQ ID NO: 46 | DYTIH |
| Chain CDR | SEQ ID NO: 47 | WFYPGTVSIKYNEKFKD |
| | SEQ ID NO: 48 | HEHPHYYGDSYDAMGY |
| L105S6 Light | SEQ ID NO: 4 | RASENIYSNLA |
| Chain CDR | SEQ ID NO: 5 | GATNLAD |
| | SEQ ID NO: 49 | QHFWGTPYT |
| L105S6 Heavy | SEQ ID NO: 50 | QVQLQQSGTELVKPGASVKLSCKASGYIFTDY |
| Chain Variable | | TIHWVKQRSGQGLEWIGWFYPGTVSIKYNEKF |
| Region | | KDKATLTADRSSSIVYMELSRLTSEDSGVYFC |
| (mIgG2a) | | ARHEHPHYYGDSYDAMGYWGQGTSVTVSS |
| L105S6 Light | SEQ ID NO: 51 | DIQMTQSPASLSVSVGETVTITCRASENIYSNL |
| Chain Variable | | AWYQQKQGKSPQLLVYGATNLADGVPSRFG |
| Region (kappa) | | GSGSGTQYSLKINSLQPEDFGSYYCQHFWGTP |
| | | YTFGGGTKLEIT |
| L178S4 Heavy | SEQ ID NO: 52 | DNYLQ |
| Chain CDR | SEQ ID NO: 53 | PGSGNTYYSDNFTG |
| | SEQ ID NO: 54 | STVVYFDV |
| L178S4 Light | SEQ ID NO: 55 | SNYAN |
| Chain CDR | SEQ ID NO: 56 | GTNNRAP |
| | SEQ ID NO: 57 | WYSNHWV |
| L178S4 Heavy | SEQ ID NO: 58 | QVQLQQSGPELVKPGASVKISCKASGYIFTDN |
| Chain Variable | | YLQWVKQRPGQGLEWIGWIFPGSGNTYYSDN |
| Region | | FTGKATLTVDKSSITAYMLLSSLTSEDSAVYFC |
| (mIgG2a) | | SRSTVVYFDVWGTGTTVTVSS |
| L178S4 Light | SEQ ID NO: 59 | QAVVTQESALTTSPGETVTLTCRSSTGTVTTSN |
| Chain Variable | | YANWVQEKPDHLFTGLIGGTNNRAPGVPARF |
| Region | | SGSLIGDQAALTITGAQTEDEAIYFCALWYSN |
| (lambda) | | HWVFGGGTKLTVL |
| L196S3 Heavy | SEQ ID NO: 60 | DYGMH |
| Chain CDR | SEQ ID NO: 61 | YISSDSSTIYYADTVKG |
| | SEQ ID NO: 62 | RAAQGYVMDY |
| L196S3 Light | SEQ ID NO: 63 | KASQSVSDDVA |
| Chain CDR | SEQ ID NO: 64 | ASNRYT |
| | SEQ ID NO: 65 | QQDYGSPT |
| L196S3 Heavy | SEQ ID NO: 66 | EVOLVESGGGLVKPGGSLKLSCAASGFTFSDY |
| Chain Variable | | GMHWVRQAPEKRLEWVAYISSDSSTIYYADT |
| Region | | VKGRFTISRDNAKNTLFLEMTSLRSEDTAMYY |
| (mIgG2a) | | CARRAAQGYVMDYWGQGTSVTVSS |
| L196S3 Light | SEQ ID NO: 67 | SIVMTQTPKFLLVSAGDRVTITCKASQSVSDD |
| Chain Variable | | VAWYQQKPGQSPKLLIYYASNRYTGVPDRFT |
| Region (kappa) | | GSGYGTDFTFTISTVQAEDLAVYFCQQDYGSP |
| | | TFGGGTKLEIK |
| L207S3 Heavy | SEQ ID NO: 68 | TYGMS |
| Chain CDR | SEQ ID NO: 69 | WINTYSGEPTYADEFKG |
| | SEQ ID NO: 70 | RGYDGYYYTMDY |
| L207S3 Light | SEQ ID NO: 4 | RASENIYSNLA |
| Chain CDR | SEQ ID NO: 71 | AATNLAD |
| | SEQ ID NO: 72 | QHFFGAPWT |
| L207S3 Heavy | SEQ ID NO: 73 | QIQLVQSGPELKKPGETVKISCKASGYTFTTYG |
| Chain Variable | | MSWVKQAPGKGLKWMAWINTYSGEPTYADE |
| Region | | FKGRFAFSLETSVSTAYLQINNLKNEDTATYFC |
| (mIgG1) | | ARRGYDGYYYTMDYWGQGTSVTVSS |
| L207S3 Light | SEQ ID NO: 74 | DIQMTQSPASLSASVGETVTITCRASENIYSNL |
| Chain Variable | | AWYQQKQGKSPQLLVSAATNLADGVPSRFSG |
| Region (kappa) | | SGSGTQFSLKINSLQPEDFGSYYCQHFFGAPW |
| | | TFGGGTKLEIK |
| CDR-H1 | | X₁X₂X₃X₄N |
| | | wherein X₁ is D or G, X₂ is Y or S, X₃ is Y, S, or F, |
| | | and X₄ is M or V. |
| CDR-H2 | SEQ ID NO: 76 | X₁IX₂PX₃X₄X₅X₆X₇X₈X₉NX₁₀KFKX₁₁ |
| | | wherein X₁ is R, D, or Y; X₂ is N or Y; X₃ is G, N, |
| | | D, or Y; X₄ is V or N; X₅ is G or Y; X₆ is R, G, V, |
| | | or D; X₇ is I or T; X₈ is H, S, R, of F; X₉ is Y or N; |
| | | X₁₀ is E or Q; X₁₁ is D or G. |
| CDR-H3 | | X₁X₂X₃X₄X₅X₆X₇X₈X₉X₁₀X₁₁X₁₂X₁₃X₁₄X₁₅ |
| | | wherein X₁ is G, S, or E; X₂ is G, T, or I; X₃ is A, T, |
| | | or Y; X₄ E, V, Y, or I; X₅ is L, G, T, or A; X₆ is T, G, |
| | | or A; X₇ is G, A, T, or D; X₈ is T, W, Y, L, or S; X₉ |
| | | is Y, F, E, or S; X₁₀ is W, A, E, or D; X₁₁ is Y, D, F, |
| | | or V; X₁₂ is F, A, or absent; X₁₃ is D, M, Y, or |
| | | absent; X₁₄ is V, D, or absent; and X₁₅ is Y or |
| | | absent. |
| CDR-L1 | SEQ ID NO: 78 | RASEX₁X₂X₃X₄X₅X₆X₇X₈X₉ X₁₀ |
| | | wherein X₁ is N or S; X₂ I or V; X₃ is Y or D; X₄ is |
| | | S or N; X₅ is N or Y; X₆ is L or G; X₇ is A, Y, or N; |
| | | X₈ is S, T, or absent; X₉ is F or absent; X₁₀ is L, M, |
| | | or absent. |
| CDR-L2 | | X₁X₂X₃X₄LX₅X₆ |
| | | wherein X₁ is L, R, G, or A; X₂ is A or S; X₃ is S or |
| | | T; X₄ is N or S; X₅ is E or A; and X₆ is S or D. |
| CDR-L3 | | X₁QX₂X₃X₄DX₅X₆X₇T |
| | | wherein X₁ is absent or Q; X₂ is S, H, N, or Q; X₃ is |
| | | N, F, or S; X₄ E, W, or S; X₅ is L, S, T, or H; X₆ s M |
| | | or P; and X₇ is Y, F, or L. |
| Human IgG1 | SEQ ID NO: 81 | ASTKGPSVFPLAPSSKSTSGGTAALGCLVKDY |
| | | FPEPVTVSWNSGALTSGVHTFPAVLQSSGLYS |
| | | LSSVVTVPSSSLGTQTYICNVNHKPSNTKVDK |
| | | KVEPKSCDKTHTCPPCPAPELLGGPSVFLFPPK |
| | | PKDTLMISRTPEVTCVVVDVSHEDPEVKFNWY |
| | | VDGVEVHNAKTKPREEQYNSTYRVVSVLTVL |
| | | HQDWLNGKEYKCKVSNKALPAPIEKTISKAK |
| | | GQPREPQVYTLPPSRDELTKNQVSLTCLVKGF |
| | | YPSDIAVEWESNGQPENNYKTTPPVLDSDGSF |
| | | FLYSKLTVDKSRWQQGNVFSCSVMHEALHNH |
| | | YTQKSLSLSPGK |
| LILRA1 | SEQ ID NO: 82 | MTPIVTVLICLRLSLGPRTHVQAGTLPKPTL |
| (#O75019) | | WAEPGSVITQGSPVTLWCQGILETQEYRLY |
| *Does not | | REKKTAPWITRIPQEIVKKGQFPIPSITWEH |
| include C- | | TGRYRCFYGSHTAGWSEPSDPLELVVTGAY |
| terminus 6x His | | IKPTLSALPSPVVTSGGNVTLHCVSQVAFGS |
| tag | | FILCKEGEDEHPQCLNSQPRTHGWSRAIFSV |
| | | GPVSPSRRWSYRCYAYDSNSPHVWSLPSDL |
| | | LELLVLGVSKKPSLSVQPGPIVAPGESLTLQ |
| | | CVSDVSYDRFVLYKEGERDFLQLPGPQPQA |
| | | GLSQANFTLGPVSRSYGGQYRCSGAYNLSS |
| | | EWSAPSDPLDILIAGQFRGRPFISVHPGPTVA |
| | | SGENVTLLCQSWGPFHTFLLTKAGAADAPL |
| | | RLRSIHEYPKYQAEFPMSPVTSAHSGTYRCY |
| | | GSLSSNPYLLSHPSDSLELMVSGAAETLSPP |
| | | QNKSDSKAGAANTLSPSQNKTASHPQDYTV |
| | | ENLIRMGIAGLVLVVLGILLFEAQHSQRSL |
| LILRA2 | SEQ ID NO: 83 | MTPILTVLICLGLSLGPRTHVQAGHLPKPTLW |
| (#NP_001124389) | | AEPGSVIIQGSPVTLRCQGSLQAEEYHLYRE |
| *Does not | | NKSASWVRRIQEPGKNGQFPIPSITWEHAG |
| include C- | | RYHCQYYSHNHSSEYSDPLELVVTGAYSKP |
| terminus 6x His | | TLSALPSPVVTSGGNVTLQCVSQVAFDGFIL |
| tag | | CKEGEDEHPQRLNSHSHARGWSWAIFSVGP |
| | | VSPSRRWSYRCYAYDSNSPYVWSLPSDLLEL |
| | | LVPGVSKKPSLSVQPGPMVAPGESLTLQCV |
| | | SDVGYDRFVLYKEGERDFLQRPGWQPQAG |
| | | LSQANFTLGPVSPSHGGQYRCYSAHNLSSE |
| | | WSAPSDPLDILITGQFYDRPSLSVQPVPTVAP |
| | | GKNVTLLCQSRGQFHTFLLTKEGAGHPPLH |
| | | LRSEHQAQQNQAEFRMGPVTSAHVGTYRC |
| | | YSSLSSNPYLLSLPSDPLELVVSEAAETLSPS |
| | | QNKTDSTTTSLGQHPQDYTVENLIRMGVAG |
| | | LVLVVLGILLFEAQHSQRSLQDAAGR |
| LILRA3 | SEQ ID NO: 84 | MTSILTVLICLGLSLDPRTHVQAGPLPKPTLW |
| (#AAH28208.1) | | AEPGSVITQGSPVTLRCQGSLETQEYHLYRE |
| *Does not | | KKTALWITRIPQELVKKGQFPILSITWEHAG |
| include C- | | RYCCIYGSHTVGLSESSDPLELVVTGAYSKP |
| terminus 6x His | | TLSALPSPVVTSGGNVTIQCDSQVAFDGFIL |
| tag and linker | | CKEGEDEHPQCLNSHSHARGSSRAIFSVGPV |
| | | SPSRRWSYRCYGYDSRAPYVWSLPSDLLGL |
| | | LVPGVSKKPSLSVQPGPVVAPGEKLTFQCG |
| | | SDAGYDRFVLYKEWGRDFLQRPGRQPQAG |
| | | LSQANFTLGPVSRSYGGQYTCSGAYNLSSE |
| | | WSAPSDPLDILITGQIRARPFLSVRPGPTVAS |
| | | GENVTLLCQSQGGMHTFLLTKEGAADSPL |
| | | RLKSKRQSHKYQAEFPMSPVTSAHAGTYRC |
| | | YGSLSSNPYLLTHPSDPLELVVSGAAETLSPP |
| | | QNKSDSKAGE |
| LILRA4 | SEQ ID NO: 85 | MTLILTSLLFFGLSLGPRTRVQAENLPKPILW |
| (#P59901) | | AEPGPVITWHNPVTIWCQGTLEAQGYRLDK |
| *Does not | | EGNSMSRHILKTLESENKVKLSIPSMMWEH |
| include C- | | AGRYHCYYQSPAGWSEPSDPLELVVTAYSR |
| terminus 6x | | PTLSALPSPVVTSGVNVTLRCASRLGLGRFT |
| His tag | | LIEEGDHRLSWTLNSHQHNHGKFQALFPM |
| | | GPLTFSNRGTFRCYGYENNTPYVWSEPSDP |
| | | LQLLVSGVSRKPSLLTLQGPVVTPGENLTL |
| | | QCGSDVGYIRYTLYKEGADGLPQRPGRQPQ |
| | | AGLSQANFTLSPVSRSYGGQYRCYGAHNVS |
| | | SEWSAPSDPLDILIAGQISDRPSLSVQPGPTV |
| | | TSGEKVTLLCQSWDPMFTFLLTKEGAAHPP |
| | | LRLRSMYGAHKYQAEFPMSPVTSAHAGTY |
| | | RCYGSRSSNPYLLSHPSEPLELVVSGATETL |
| | | NPAQKKSDSKTAPHLQDYTVENLIRMGVAG |
| | | LVLLFLGILLFEAQHSQRSPPRCSQEANSRKDN |
| | | APFRVVEPWEQI |
| LILRA5 | SEQ ID NO: 86 | MAPWSHPSAQLQPVGGDAVSPALMVLLCLGL |
| (#A6NI73) | | SLGPRTHVQAGNLSKATLWAEPGSVISRGNS |
| *Does not | | VTIRCQGTLEAQEYRLVKEGSPEPWDTQNP |
| include C- | | LEPKNKARFSIPSMTEHHAGRYRCYYYSPA |
| terminus 6x His | | GWSEPSDPLELVVTGFYNKPTLSALPSPVVT |
| tag | | SGENVTLQCGSRLRFDRFILTEEGDHKLSW |
| | | TLDSQLTPSGQFQALFPVGPVTPSHRWMLR |
| | | CYGSRRHILQVWSEPSDLLEIPVSGAADNLS |
| | | PSQNKSDSGTASHLQDYAVENLIRMGMAGLI |
| | | LVVLGILIFQDWHSQRSPQAAAGR |
| LILRA6 | SEQ ID NO: 87 | MTPALTALLCLGLSLGPRTRVQAGPFPKPTL |
| (#Q6PI73) | | WAEPGSVISWGSPVTIWCQGSLEAQEYQLD |
| *Does not | | KEGSPEPLDRNNPLEPKNKARFSIPSMTQHH |
| include C- | | AGRYRCHYYSSAGWSEPSDPLELVMTGFYN |
| terminus 6x His | | KPTLSALPSPVVASGGNMTLRCGSQKGYHH |
| tag | | FVLMKEGEHQLPRTLDSQQLHSGGFQALFP |
| | | VGPVTPSHRWRFTCYYYYTNTPRVWSHPSD |
| | | PLEILPSGVSRKPSLLTLQGPVLAPGQSLTL |
| | | QCGSDVGYDRFVLYKEGERDFLQRPGQQP |
| | | QAGLSQANFTLGPVSPSHGGQYRCYGAHNL |
| | | SSEWSAPSDPLNILMAGQIYDTVSLSAQPGP |
| | | TVASGENVTLLCQSRGYFDTFLLTKEGAAH |
| | | PPLRLRSMYGAHKYQAEFPMSPVTSAHAGT |
| | | YRCYGSYSSNPHLLSFPSEPLELMVSGHSGG |
| | | SSLPPTGPPSTPASHAKDYTVENLIRMGMAG |
| | | LVLVFLGILLFEAQHSQRNPQDAAGR |
| LILRB1 | SEQ ID NO: 88 | MTPILTVLICLGLSLGPRTHVQAGHLPKPTLW |
| (#Q8NHL6) | | AEPGSVITQGSPVTLRCQGGQETQEYRLYR |
| *Does not | | EKKTALWITRIPQELVKKGQFPIPSITWEHA |
| include C- | | GRYRCYYGSDTAGRSESSDPLELVVTGAYI |
| terminus 6x His | | KPTLSAQPSPVVNSGGNVILQCDSQVAFDGF |
| tag | | SLCKEGEDEHPQCLNSQPHARGSSRAIFSVG |
| | | PVSPSRRWWYRCYAYDSNSPYEWSLPSDLL |
| | | ELLVLGVSKKPSLSVQPGPIVAPEETLTLQC |
| | | GSDAGYNRFVLYKDGERDFLQLAGAQPQA |
| | | GLSQANFTLGPVSRSYGGQYRCYGAHNLSS |
| | | EWSAPSDPLDILIAGQFYDRVSLSVQPGPTV |
| | | ASGENVTLLCQSQGWMQTFLLTKEGAADD |
| | | PWRLRSTYQSQKYQAEFPMGPVTSAHAGT |
| | | YRCYGSQSSKPYLLTHPSDPLELVVSGPSGG |
| | | PSSPTTGPTSTSGPEDQPLTPTGSDPQSGLG |
| | | RHLGVVIGILVAVILLLLLLLLLFLILRHRRQG |
| | | KHWTSTQRKADFQHPAGAVGPEPTDRGLQW |
| | | RSSPAADAQEENLYAAVKHTQPEDGVEMDTR |
| | | SPHDEDPQAVTYAEVKHSRPRREMASPPSPLS |
| | | GEFLDTKDRQAEEDRQMDTEAAASEAPQDVT |
| | | YAQLHSLTLRREATEPPPSQEGPSPAVPSIYAT |
| | | LAIH |
| LILRB2 | SEQ ID NO: 89 | MTPIVTVLICLGLSLGPRTHVQTGTIPKPTLW |
| (#AAH36827) | | AEPDSVITQGSPVTLSCQGSLEAQEYRLYRE |
| *Does not | | KKSASWITRIRPELVKNGQFHIPSITWEHTG |
| include C- | | RYGCQYYSRARWSELSDPLVLVMTGAYPK |
| terminus 6x His | | PTLSAQPSPVVTSGGRVTLQCESQVAFGGFI |
| tag | | LCKEGEDEHPQCLNSQPHARGSSRAIFSVGP |
| | | VSPNRRWSHRCYGYDLNSPYVWSSPSDLLE |
| | | LLVPGVSKKPSLSVQPGPVVAPGESLTLQCV |
| | | SDVGYDRFVLYKEGERDLRQLPGRQPQAG |
| | | LSQANFTLGPVSRSYGGQYRCYGAYNLSSE |
| | | WSAPSDPLDILITGQIHGTPFISVQPGPTVAS |
| | | GENVTLLCQSWRQFHTFLLTKAGAADAPL |
| | | RLRSIHEYPKYQAEFPMSPVTSAHAGTYRC |
| | | YGSLNSDPYLLSHPSEPLELVVSGPSMGSSPP |
| | | PTGPISTPAGPEDQPLTPTGSDPQSGLGRHL |
| | | GVVIGILVAVVLLLLLLLLLFLILRHRRQGKH |
| | | WTSTQRKADFQHPAGAVGPEPTDRGLQWRSS |
| | | PAADAQEENLYAAVKDTQPEDGVEMDTRAA |
| | | ASEAPQDVTYAQLHSLTLRRKATEPPPSQEGE |
| | | PPAEPSIYATLAIH |
| LILRB3 | SEQ ID NO: 90 | MTPALTALLCLGLSLGPRTRVQAGPFPKPTL |
| (#AAB68668) | | WAEPGSVISWGSPVTIWCQGSLEAQEYRLD |
| *Does not | | KEGSPEPLDRNNPLEPKNKARFSIPSMTEHH |
| include C- | | AGRYRCHYYSSAGWSEPSDPLELVMTGFYN |
| terminus 6x His | | KPTLSALPSPVVASGGNMTLRCGSQKGYHH |
| tag | | FVLMKEGEHQLPRTLDSQQLHSGGFQALFP |
| | | VGPVNPSHRWRFTCYYYYMNTPQVWSHPS |
| | | DPLEILPSGVSRKPSLLTLQGPVLAPGQSLT |
| | | LQCGSDVGYDRFVLYKEGERDFLQRPGQQ |
| | | PQAGLSQANFTLGPVSPSHGGQYRCYGAHN |
| | | LSSEWSAPSDPLNILMAGQIYDTVSLSAQPG |
| | | PTVASGENVTLLCQSWWQFDTFLLTKEGA |
| | | AHPPLRLRSMYGAHKYQAEFPMSPVTSAHA |
| | | GTYRCYGSYSSNPHLLSFPSEPLELMVSGHS |
| | | GGSSLPPTGPPSTPGLGRYLEVLIGVSVAFVL |
| | | LLFLLLFLLLRRQRHSKHRTSDQRKTDFQRPA |
| | | GAAETEPKDRGLLRRSSPAADVQEENLYAAV |
| | | KDTQSEDRVELDSQSPHDEDPQAVTYAPVKHS |
| | | SPRREMASPPSSLSGEFLDTKDRQVEEDRQMD |
| | | TEAAASEASQDVTYAQLHSLTLRRKATEPPPS |
| | | QEGEPPAEPSIYATLAIH |
| LILRB4 | SEQ ID NO: 91 | MIPTFTALLCLGLSLGPRTDMQAGPLPKPTL |
| (#AAH26309) | | WAEPGSVISWGNSVTIWCQGTLEAREYRLD |
| *Does not | | KEESPAPWDRQNPLEPKNKARFSIPSMTED |
| include C- | | YAGRYRCYYRSPVGWSQPSDPLELVMTGA |
| terminus 6x His | | YSKPTLSALPSPLVTSGKSVTLLCQSRSPMD |
| tag | | TFLLIKERAAHPLLHLRSEHGAQQHQAEFP |
| | | MSPVTSVHGGTYRCFSSHGFSHYLLSHPSDP |
| | | LELIVSGSLEGPRPSPTRSVSTAAGPEDQPL |
| | | MPTGSVPHSGLRRHWEVLIGVLVVSILLLSLL |
| | | LFLLLQHWRQGKHRTLAQRQADFQRPPGAAE |
| | | PEPKDGGLQRRSSPAADVQGENFCAAVKNTQ |
| | | PEDGVEMDTRQSPHDEDPQAVTYAKVKHSRP |
| | | RREMASPPSPLSGEFLDTKDRQAEEDRQMDTE |
| | | AAASEAPQDVTYARLHSFTLRQKATEPPPSQE |
| | | GASPAEPSVYATLAIH |
| Predicted | SEQ ID NO: 92 | MTPILMVLICLGLSLGPRTHVQAGTLPKPML |
| Cynomolgous | | WAEPGSMISEGSPVTLRCQGSLQVQEYRLY |
| LILRB2 | | KGKKPASWVRRIQQELVKKGYFAIGFITWE |
| (#BAE87348.1) | | HTGQYRCQYYSHSWWSEPSDPLELVVTGA |
| *Does not | | YSKPTLSALPSPVVASGGNVSLQCDSQVTFD |
| include C- | | GFVLCKEGEDEHPQRLNSQSHARGWSWAV |
| terminus 6x his | | FSVVPVSPSRRWSYRCYGYDSHSPYVWSLPS |
| tag | | DLLELLVPGVSKKPSLSVQPGPVVAPGESLT |
| | | LQCGSDVGYDRFALYKEGERDFLQRPSRQP |
| | | QAGLSQANFLLGPVSRSHGGQYRCSGAHNL |
| | | SSEWSAPSDPLDILISGQIRGTPFLSVQPGPT |
| | | VVSGENVTLLCQSSWQFHAFLLTQAGAADA |
| | | HLHLRSMYKYPKYQAELSMSPVTSAHAGT |
| | | YRCYGSLSSNPYLLSVPSDPLELVVSGAVDT |
| | | LGLSQNNSETKTASHSQDYTVENLIRMGMA |
| | | GLILVVLGILLFKPQYSQRSLQDAARR |
| Human IgG1 | SEQ ID NO: 93 | ASTKGPSVFPLAPSSKSTSGGTAALGCLVKDY |
| L234A/L235A | | FPEPVTVSWNSGALTSGVHTFPAVLQSSGLYS |
| | | LSSVVTVPSSSLGTQTYICNVNHKPSNTKVDK |
| | | KVEPKSCDKTHTCPPCPAPEAAGGPSVFLFPPK |
| | | PKDTLMISRTPEVTCVVVDVSHEDPEVKFNWY |
| | | VDGVEVHNAKTKPREEQYNSTYRVVSVLTVL |
| | | HQDWLNGKEYKCKVSNKALPAPIEKTISKAK |
| | | GQPREPQVYTLPPSRDELTKNQVSLTCLVKGF |
| | | YPSDIAVEWESNGQPENNYKTTPPVLDSDGSF |
| | | FLYSKLTVDKSRWQQGNVFSCSVMHEALHNH |
| | | YTQKSLSLSPGK |
| Human IgG1 | SEQ ID NO: 94 | ASTKGPSVFPLAPSSKSTSGGTAALGCLVKDY |
| L234A/L235A/ | | FPEPVTVSWNSGALTSGVHTFPAVLQSSGLYS |
| P329G | | LSSVVTVPSSSLGTQTYICNVNHKPSNTKVDK |
| | | KVEPKSCDKTHTCPPCPAPEAAGGPSVFLFPPK |
| | | PKDTLMISRTPEVTCVVVDVSHEDPEVKFNWY |
| | | VDGVEVHNAKTKPREEQYNSTYRVVSVLTVL |
| | | HQDWLNGKEYKCKVSNKALGAPIEKTISKAK |
| | | GQPREPQVYTLPPSRDELTKNQVSLTCLVKGF |
| | | YPSDIAVEWESNGQPENNYKTTPPVLDSDGSF |
| | | FLYSKLTVDKSRWQQGNVFSCSVMHEALHNH |
| | | YTQKSLSLSPGK |
| HuL45 | SEQ ID NO: 95 | QVQLVQSGA EVKKPGASVK VSCKASGYTF |
| HCVR | | TDYYMNWVRQ APGQRLEWIG |
| | | DINPNNGGTSY NQKFKGRATI TVDTSASTAY |
| | | MELSSLRSEDTAV YYCARGGAEL |
| | | TGTYWYFDVWGQGTTV TVSS |
| HuL45 | SEQ ID NO: 96 | QVQLVQSGA EVKKPGASVK VSCKASGYTF |
| HCVR | | TDYYMNWVRQ APGORLEWIG |
| | | DINPNNGGTSY NQKFKGRATI TVDKSASTAY |
| | | MELSSLRSEDTAV YYCARGGAEL |
| | | TGTYWYFDVWGQGTTV TVSS |
| HuL45 | SEQ ID NO: 97 | QVQLVQSGA EVKKPGASVK VSCKASGYTF |
| HCVR | | TDYYMNWVRQ APGQSLEWIG |
| | | DINPNNGGTSY NQKFKGRATI TVDTSASTAY |
| | | MELSSLRSEDTAV YYCARGGAEL |
| | | TGTYWYFDVWGQGTTV TVSS |
| HuL45 | SEQ ID NO: 98 | DIQMTQSPS SLSASVGDRV TITCRASENI |
| LCVR | | YSNLAWYQQK PGKAPKVLVY |
| | | GATNLADGVP SRFSGSGSGT EYTLTISSLQ |
| | | PEDFATYYCQ HFWDSPFTFG GGTKVEIK |
| HuL45 | SEQ ID NO: 99 | DIQMTQSPS SLSASVGDRV TITCRASENI |
| LCVR | | YSNLAWYQQK PGKAPKVLVY |
| | | GATNLADGVP SRFSGSGSGT EFTLTISSLQ |
| | | PEDFATYYCQ HFWDSPFTFG GGTKVEIK |
| HuL77 | SEQ ID NO: 100 | QVQLVQSGA EVKKPGSSVK VSCKASGYSF |
| HCVR | | TGYSVNWVRQ APGQGLEWIG RINPYNGDIFN |
| | | NQKFKGRATI TVDKSTSTAY |
| | | MELSSLRSEDTAV YYCARGTTVG |
| | | GAWFAYWGQGTLV TVSS |
| HuL77 | SEQ ID NO: 101 | QVQLVQSGA EVKKPGSSVK VSCKASGYSF |
| HCVR | | TGYSVNWVKQ APGQGLEWIG RINPYNGDIFN |
| | | NQKFKGRATI TVDKSTSTAY |
| | | MELSSLRSEDTAV YYCARGTTVG |
| | | GAWFAYWGQGTLV TVSS |
| HuL77 | SEQ ID NO: 102 | QVQLVQSGA EVKKPGSSVK VSCKASGYSF |
| HCVR | | TGYSVNWVRQ RPGQGLEWIG RINPYNGDIFN |
| | | NQKFKGRATI TVDKSTSTAY |
| | | MELSSLRSEDTAV YYCARGTTVG |
| | | GAWFAYWGQGTLV TVSS |
| HuL77 | SEQ ID NO: 103 | DIVMTQSPD SLAVSLGERA |
| LCVR | | TINCRASESVDSYG YSFLHWYQQK |
| | | PGQPPKLLIY LASNLESGIP DRFSGSGSGT |
| | | DFTLTISSLQ AEDVAVYYCQ QSNEDLMYTFG |
| | | GGTK VEIK |
| HuL77 | SEQ ID NO: 104 | VIVMTQSPD SLAVSLGERA |
| LCVR | | TINCRASESVDSYG YSFLHWYQQK |
| | | PGQPPKLLIY LASNLESGIP DRFSGSGSGT |
| | | DFTLTISSLQ AEDVAVYYCQ QSNEDLMYTFG |
| | | GGTK VEIK |
| HuL156 | SEQ ID NO: 105 | QVQLVQSGA EVKKPGSSVK VSCKASGYTF |
| HCVR | | TDYYMNWVRQ APGOGLEWIA |
| | | RIYPGVYRTHY NEKFKDRATL TADKSTSTAY |
| | | MELSSLRSEDTAV YYCARSGYYG |
| | | GTYEEDAMDYWGQGTTV TVSS |
| HuL156 | SEQ ID NO: 106 | QVQLVQSGA EVKKPGSSVK VSCKASGYTF |
| HCVR | | TDYYMNWVRQ APGQGLEWIA |
| | | RIYPGVYRTHY NEKFKDRVTL TADKSTSTAY |
| | | MELSSLRSEDTAV YYCARSGYYG |
| | | GTYEEDAMDYWGQGTTV TVSS |
| HuL156 | SEQ ID NO: 107 | DIQMTQSPS SLSASVGDRV TITCRASENI |
| LCVR | | YSNLAWYQQK PGKAPKLLVY AATSLADGVP |
| | | SRFSGSGSGT DYTLTISSLQ PEDFATYYCQ |
| | | NFWDTPYTFG QGTKVEIK |
| HuL156 | SEQ ID NO: 108 | DIQMTQSPS SLSASVGDRV TITCRASENI |
| LCVR | | YSNLAWYQQK PGKAPKLLVY AATSLADGVP |
| | | SRFRGSGSGT DYTLTISSLQ PEDFATYYCQ |
| | | NFWDTPYTFG QGTKVEIK |
| HuL180 | SEQ ID NO: 109 | QVQLVQSGA EVKKPGSSVK VSCKASGYSF |
| HCVR | | IGYFMNWMRQ APGQGLEWIG RINPYNGDIFY |
| | | NQKFKGRATI TVDKSTSTAY |
| | | MELSSLRSEDTAV YYCARGITVA |
| | | AGSFDVWGQGTTV TVSS |
| HuL180 | SEQ ID NO: 110 | QVQLVQSGA EVKKPGSSVK VSCKASGYSF |
| HCVR | | IGYFMNWMRQ APGQGLEWIG RINPYNGDIFY |
| | | NQKFKGRVTI TVDKSTSTAY |
| | | MELSSLRSEDTAV YYCARGITVA |
| | | AGSFDVWGQGTTV TVSS |
| HuL 180 | SEQ ID NO: 111 | DIVMTQSPD SLAVSLGERA |
| LCVR | | TINCRASESVDNYG NTFMHWYQQK |
| | | PGQPPKLLIY RASNLESGIP DRFSGSGSKT |
| | | DFTLTISSLQ AEDVAVYYCQ QSSDHPLTFG |
| | | GGTK VEIK |
| HuL 180 | SEQ ID NO: 112 | DIVMTQSPD SLAVSLGERA |
| LCVR | | TINCRASESVDNYG NTFMHWYQQK |
| | | PGQPPKLLIY RASNLESGIP DRFSGSGSGT |
| | | DFTLTISSLQ AEDVAVYYCQ QSSDHPLTFG |
| | | GGTKVEIK |
| HC CDR1 | SEQ ID NO: 1 | DYYMN |
| HC CDR2 | SEQ ID NO: 113 | IYPGVYRT |
| HC CDR3 | SEQ ID NO: 18 | SGYYGGTYEEDAMDY |
| LC CDR1 | SEQ ID NO: 114 | ENIYSN |
| LC CDR2 | | AAT |
| LC CDR3 | SEQ ID NO: 20 | QNFWDTPYT |
| HC CDR1 | SEQ ID NO: 1 | DYYMN |
| HC CDR2 | SEQ ID NO: 116 | INPNNGGT |
| HC CDR3 | SEQ ID NO: 3 | GGAELTGTYWYFDV |
| LC CDR1 | SEQ ID NO: 117 | ENIYS |
| LC CDR2 | | GAT |
| LC CDR3 | SEQ ID NO: 6 | QHFWDSPFT |
| HC CDR1 | SEQ ID NO: 9 | GYSVN |
| HC CDR2 | SEQ ID NO: 119 | INPYNGDI |
| HC CDR3 | SEQ ID NO: 11 | GTTVGGAWFAY |
| LC CDR1 | SEQ ID NO: 120 | ESVDSYGYSF |
| LC CDR2 | | LAS |
| LC CDR3 | SEQ ID NO: 14 | QQSNEDLMYT |
| HC CDR1 | SEQ ID NO: 30 | GYFMN |
| HC CDR2 | SEQ ID NO: 119 | INPYNGDI |
| HC CDR3 | SEQ ID NO: 32 | GITVAAGSFDV |
| LC CDR1 | SEQ ID NO: 122 | ESVDNYGNTF |
| LC CDR2 | | RAS |
| LC CDR3 | SEQ ID NO: 35 | QQSSDHPLT |
| Hz45.01-LALA | SEQ ID NO: 124 | QVQLVQSGAEVKKPGASVKVSCKASGYTFTD |
| Antibody | | YYMNWVRQAPGQRLEWIGDINPNNGGTSYN |
| Heavy Chain | | QKFKGRATITVDTSASTAYMELSSLRSEDTAV |
| | | YYCARGGAELTGTYWYFDVWGQGTTVTVSS |
| | | ASTKGPSVFPLAPSSKSTSGGTAALGCLVKDY |
| | | FPEPVTVSWNSGALTSGVHTFPAVLQSSGLYS |
| | | LSSVVTVPSSSLGTQTYICNVNHKPSNTKVDK |
| | | KVEPKSCDKTHTCPPCPAPEAAGGPSVFLFPPK |
| | | PKDTLMISRTPEVTCVVVDVSHEDPEVKFNWY |
| | | VDGVEVHNAKTKPREEQYNSTYRVVSVLTVL |
| | | HQDWLNGKEYKCKVSNKALPAPIEKTISKAK |
| | | GQPREPQVYTLPPSRDELTKNQVSLTCLVKGF |
| | | YPSDIAVEWESNGQPENNYKTTPPVLDSDGSF |
| | | FLYSKLTVDKSRWQQGNVFSCSVMHEALHNH |
| | | YTQKSLSLSPGK |
| Hz45.01-LALA | SEQ ID NO: 125 | DIQMTQSPSSLSASVGDRVTITCRASENIYSNL |
| Antibody Light | | AWYQQKPGKAPKVLVYGATNLADGVPSRFSG |
| Chain | | SGSGTEYTLTISSLQPEDFATYYCQHFWDSPFT |
| | | FGGGTKVEIKRTVAAPSVFIFPPSDEQLKSGTA |
| | | SVVCLLNNFYPREAKVQWKVDNALQSGNSQE |
| | | SVTEQDSKDSTYSLSSTLTLSKADYEKHKVYA |
| | | CEVTHQGLSSPVTKSFNRGEC |
| Hz77-LALA | SEQ ID NO: 126 | QVQLVQSGAEVKKPGSSVKVSCKASGYSFTG |
| Antibody | | YSVNWVRQAPGQGLEWIGRINPYNGDIFNNQ |
| Heavy Chain | | KFKGRATITVDKSTSTAYMELSSLRSEDTAVY |
| | | YCARGTTVGGAWFAYWGQGTLVTVSSASTK |
| | | GPSVFPLAPSSKSTSGGTAALGCLVKDYFPEPV |
| | | TVSWNSGALTSGVHTFPAVLQSSGLYSLSSVV |
| | | TVPSSSLGTQTYICNVNHKPSNTKVDKKVEPK |
| | | SCDKTHTCPPCPAPEAAGGPSVFLFPPKPKDTL |
| | | MISRTPEVTCVVVDVSHEDPEVKFNWYVDGV |
| | | EVHNAKTKPREEQYNSTYRVVSVLTVLHQDW |
| | | LNGKEYKCKVSNKALPAPIEKTISKAKGQPRE |
| | | PQVYTLPPSRDELTKNQVSLTCLVKGFYPSDIA |
| | | VEWESNGQPENNYKTTPPVLDSDGSFFLYSKL |
| | | TVDKSRWQQGNVFSCSVMHEALHNHYTQKS |
| | | LSLSPGK |
| Hz77-LALA | SEQ ID NO: 127 | DIVMTQSPDSLAVSLGERATINCRASESVDSYG |
| Antibody Light | | YSFLHWYQQKPGQPPKLLIYLASNLESGIPDRF |
| Chain | | SGSGSGTDFTLTISSLQAEDVAVYYCQQSNED |
| | | LMYTFGGGTKVEIKRTVAAPSVFIFPPSDEQLK |
| | | SGTASVVCLLNNFYPREAKVQWKVDNALQSG |
| | | NSQESVTEQDSKDSTYSLSSTLTLSKADYEKH |
| | | KVYACEVTHQGLSSPVTKSFNRGEC |
| Hz156.03- | SEQ ID NO: 128 | QVQLVQSGAEVKKPGSSVKVSCKASGYTFTD |
| LALA | | YYMNWVRQAPGQGLEWIARIYPGVYRTHYN |
| Antibody | | EKFKDRVTLTADKSTSTAYMELSSLRSEDTAV |
| Heavy Chain | | YYCARSGYYGGTYEEDAMDYWGQGTTVTVS |
| | | SASTKGPSVFPLAPSSKSTSGGTAALGCLVKD |
| | | YFPEPVTVSWNSGALTSGVHTFPAVLQSSGLY |
| | | SLSSVVTVPSSSLGTQTYICNVNHKPSNTKVD |
| | | KKVEPKSCDKTHTCPPCPAPEAAGGPSVFLFPP |
| | | KPKDTLMISRTPEVTCVVVDVSHEDPEVKFNW |
| | | YVDGVEVHNAKTKPREEQYNSTYRVVSVLTV |
| | | LHQDWLNGKEYKCKVSNKALPAPIEKTISKAK |
| | | GQPREPQVYTLPPSRDELTKNQVSLTCLVKGF |
| | | YPSDIAVEWESNGQPENNYKTTPPVLDSDGSF |
| | | FLYSKLTVDKSRWQQGNVFSCSVMHEALHNH |
| | | YTQKSLSLSPGK |
| Hz156.03- | SEQ ID NO: 129 | DIQMTQSPSSLSASVGDRVTITCRASENIYSNL |
| LALA | | AWYQQKPGKAPKLLVYAATSLADGVPSRFSG |
| Antibody Light | | SGSGTDYTLTISSLQPEDFATYYCQNFWDTPY |
| Chain | | TFGQGTKVEIKRTVAAPSVFIFPPSDEQLKSGT |
| | | ASVVCLLNNFYPREAKVQWKVDNALQSGNSQ |
| | | ESVTEQDSKDSTYSLSSTLTLSKADYEKHKVY |
| | | ACEVTHQGLSSPVTKSFNRGEC |
| Hz180-LALA | SEQ ID NO: 130 | QVQLVQSGAEVKKPGSSVKVSCKASGYSFIGY |
| Antibody | | FMNWMRQAPGQGLEWIGRINPYNGDIFYNQK |
| Heavy Chain | | FKGRVTITVDKSTSTAYMELSSLRSEDTAVYY |
| | | CARGITVAAGSFDVWGQGTTVTVSSASTKGPS |
| | | VFPLAPSSKSTSGGTAALGCLVKDYFPEPVTVS |
| | | WNSGALTSGVHTFPAVLQSSGLYSLSSVVTVP |
| | | SSSLGTQTYICNVNHKPSNTKVDKKVEPKSCD |
| | | KTHTCPPCPAPEAAGGPSVFLFPPKPKDTLMIS |
| | | RTPEVTCVVVDVSHEDPEVKFNWYVDGVEVH |
| | | NAKTKPREEQYNSTYRVVSVLTVLHQDWLNG |
| | | KEYKCKVSNKALPAPIEKTISKAKGQPREPQV |
| | | YTLPPSRDELTKNQVSLTCLVKGFYPSDIAVE |
| | | WESNGQPENNYKTTPPVLDSDGSFFLYSKLTV |
| | | DKSRWQQGNVFSCSVMHEALHNHYTQKSLSL |
| | | SPGK |
| Hz180-LALA | SEQ ID NO: 131 | DIVMTQSPDSLAVSLGERATINCRASESVDNY |
| Antibody Light | | GNTFMHWYQQKPGQPPKLLIYRASNLESGIPD |
| Chain | | RFSGSGSGTDFTLTISSLQAEDVAVYYCQQSSD |
| | | HPLTFGGGTKVEIKRTVAAPSVFIFPPSDEQLK |
| | | SGTASVVCLLNNFYPREAKVQWKVDNALQSG |
| | | NSQESVTEQDSKDSTYSLSSTLTLSKADYEKH |
| | | KVYACEVTHQGLSSPVTKSFNRGEC |

Bolded amino acids represent the extracellular domains.

Any of the above protocols or similar variants thereof can be described in various documentation associated with a pharmaceutical product. This documentation can include, without limitation, protocols, statistical analysis plans, investigator brochures, clinical guidelines, medication guides, risk evaluation and mediation programs, prescribing information and other documentation that may be associated with a pharmaceutical product. It is specifically contemplated that such documentation may be physically packaged with an pharmaceutical product according to the present disclosure as a kit, as may be beneficial or as set forth by regulatory authorities.

While the subject matter of this disclosure has been described and shown in considerable detail with reference to certain illustrative embodiments, including various combinations and sub-combinations of features, those skilled in the art will readily appreciate other embodiments and variations and modifications thereof as encompassed within the scope of the present disclosure. Moreover, the descriptions of such embodiments, combinations, and sub-combinations is not intended to convey that the claimed subject matter requires features or combinations of features other than those expressly recited in the claims. Accordingly, the scope of this disclosure is intended to include all modifications and variations encompassed within the spirit and scope of the following appended claims.

1. An antibody or antigen-binding fragment thereof that binds ILT4, comprising:
a heavy chain variable domain comprising:
a CDR-H1 comprising an amino acid sequence selected from the group consisting of:
(SEQ ID NO: 1)
DYYMN,
(SEQ ID NO: 9)
GYSVN,
(SEQ ID NO: 23)
DSYMN,
(SEQ ID NO: 30)
GYFMN,
(SEQ ID NO: 38)
SYWMN,
(SEQ ID NO: 46)
DYTIH,
(SEQ ID NO: 52)
DNYLQ,
(SEQ ID NO: 60)
DYGMH,
and
(SEQ ID NO: 68)
TYGMS,
a CDR-H2 comprising an amino acid sequence selected from the group consisting of:
(SEQ ID NO: 2)
DINPNNGGTSYNQKFKG,
(SEQ ID NO: 10)
RINPYNGDIFNNQKFKG,
(SEQ ID NO: 17)
RIYPGVYRTHYNEKFKD,
(SEQ ID NO: 24)
YINPDNGVTRYNQKFKG,
(SEQ ID NO: 31)
RINPYNGDIFYNQKFKG,
(SEQ ID NO: 39)
QIYPGHGDTNYNGKFKG,
(SEQ ID NO: 47)
WFYPGTVSIKYNEKFKD,
(SEQ ID NO: 53)
PGSGNTYYSDNFTG,
(SEQ ID NO: 61)
YISSDSSTIYYADTVKG,
and
(SEQ ID NO: 69)
WINTYSGEPTYADEFKG,
and
a CDR-H3 comprising an amino acid sequence selected from the group consisting of:
(SEQ ID NO: 3)
GGAELTGTYWYFDV,
(SEQ ID NO: 11)
GTTVGGAWFAY,
(SEQ ID NO: 18)
SGYYGGTYEEDAMDY,
(SEQ ID NO: 25)
EGTITTDLSWFAY,
(SEQ ID NO: 32)
GITVAAGSFDV,
(SEQ ID NO: 40)
EGSELGRLFAY,
(SEQ ID NO: 48)
HEHPHYYGDSYDAMGY,
(SEQ ID NO: 54)
STVVYFDV,
(SEQ ID NO: 62)
RAAQGYVMDY,
and
(SEQ ID NO: 70)
RGYDGYYYTMDY;
and
a light chain variable domain comprising:
a CDR-L1 comprising an amino acid sequence selected from the group consisting of:
(SEQ ID NO: 4)
RASENIYSNLA,
(SEQ ID NO: 12)
RASESVDSYGYSFLH,
(SEQ ID NO: 33)
RASESVDNYGNTFMH,
(SEQ ID NO: 41)
SASSSVSFMY,
(SEQ ID NO: 55)
SNYAN,
and
(SEQ ID NO: 63)
KASQSVSDDVA,
a CDR-L2 comprising an amino acid sequence selected from the group consisting of:
(SEQ ID NO: 5)
GATNLAD,
(SEQ ID NO: 13)
LASNLES,
(SEQ ID NO: 19)
AATSLAD,
(SEQ ID NO: 26)
ASTNLAD,
(SEQ ID NO: 34)
RASNLES,
(SEQ ID NO: 42)
LTSNLAS,
(SEQ ID NO: 53)
,
(SEQ ID NO: 56)
GTNNRAP,
(SEQ ID NO: 64)
ASNRYT,
and
(SEQ ID NO: 71)
AATNLAD,
and a CDR-L3 comprising an amino acid sequence selected from the group consisting of:
(SEQ ID NO: 6)
QHFWDSPFT,
(SEQ ID NO: 14)
QQSNEDLMYT,
(SEQ ID NO: 20)
QNFWDTPYT,
(SEQ ID NO: 27)
QHFWDTPYT,
(SEQ ID NO: 35)
QQSSDHPLT,
(SEQ ID NO: 43)
QQWSSNPPT,
(SEQ ID NO: 49)
QHFWGTPYT,
(SEQ ID NO: 57)
WYSNHWV,
(SEQ ID NO: 65)
QQDYGSPT,
and
(SEQ ID NO: 72)
QHFFGAPWT.

2. The antibody or antigen-binding fragment thereof of claim 1, wherein
the CDR-H1 has an amino acid sequence selected from the group consisting of: DYYMN (SEQ ID NO: 1), GYSVN (SEQ ID NO: 9), DSYMN (SEQ ID NO: 23), and GYFMN (SEQ ID NO: 30),
the CDR-H2 has an amino acid sequence selected from the group consisting of:
(SEQ ID NO: 2)
DINPNNGGTSYNQKFKG,
(SEQ ID NO: 10)
RINPYNGDIFNNQKFKG,
(SEQ ID NO: 17)
RIYPGVYRTHYNEKFKD,
(SEQ ID NO: 24)
YINPDNGVTRYNQKFKG,
and
(SEQ ID NO: 31)
RINPYNGDIFYNQKFKG,
and
the CDR-H3 has an amino acid sequence selected from the group consisting of:
(SEQ ID NO: 3)
GGAELTGTYWYFDV,
(SEQ ID NO: 11)
GTTVGGAWFAY,
(SEQ ID NO: 18)
SGYYGGTYEEDAMDY,
(SEQ ID NO: 25)
EGTITTDLSWFAY,
and
(SEQ ID NO: 32)
GITVAAGSFDV;
and
the CDR-L1 has an amino acid sequence selected from the group consisting of: RASENIYSNLA (SEQ ID NO: 4), RASESVDSYGYSFLH (SEQ ID NO: 12), and RASESVDNYGNTFMH (SEQ ID NO: 33),
the CDR-L2 has an amino acid sequence selected from the group consisting of:
(SEQ ID NO: 5)
GATNLAD,
(SEQ ID NO: 13)
LASNLES,
(SEQ ID NO: 19)
AATSLAD,
(SEQ ID NO: 26)
ASTNLAD,
and
(SEQ ID NO: 34)
RASNLES,
and
the CDR-L3 has an amino acid sequence selected from the group consisting of:
(SEQ ID NO: 6)
QHFWDSPFT,
(SEQ ID NO: 14)
QQSNEDLMYT,
(SEQ ID NO: 20)
QNFWDTPYT,
(SEQ ID NO: 27)
QHFWDTPYT,
(SEQ ID NO: 35)
QQSSDHPLT
and

3. The antibody or antigen-binding fragment thereof of claim 1, comprising heavy chain variable domain CDRs comprising DYYMN (SEQ ID NO: 1), DINPNNGGTSYNQKFKG (SEQ ID NO: 2), and GGAELTGTYWYFDV (SEQ ID NO: 3); and
light chain variable domain CDRs comprising RASENIYSNLA (SEQ ID NO: 4), GATNLAD (SEQ ID NO: 5), and QHFWDSPFT (SEQ ID NO: 6).

4. The antibody or antigen-binding fragment thereof of claim 1, comprising heavy chain variable domain CDRs comprising GYSVN (SEQ ID NO: 9), RINPYNGDIFNNQKFKG (SEQ ID NO: 10), and GTTVGGAWFAY (SEQ ID NO: 11); and
light chain variable domain CDRs comprising RASESVDSYGYSFLH (SEQ ID NO: 12), LASNLES (SEQ ID NO: 13), and QQSNEDLMYT (SEQ ID NO: 14).

5. The antibody or antigen-binding fragment thereof of claim 1, comprising heavy chain variable domain comprises CDRs comprising DYYMN (SEQ ID NO: 1), RIYPGVYRTHYNEKFKD (SEQ ID NO: 17), and SGYYGGTYEEDAMDY (SEQ ID NO: 18); and
light chain variable domain CDRs comprising RASENIYSNLA (SEQ ID NO: 4), AATSLAD (SEQ ID NO: 19), and QNFWDTPYT (SEQ ID NO: 20).

6. The antibody or antigen-binding fragment thereof of claim 1, comprising heavy chain variable domain CDRs comprising DSYMN (SEQ ID NO: 23), YINPDNGVTRYNQKFKG (SEQ ID NO: 24), and EGTITTDLSWFAY (SEQ ID NO: 25); and
light chain variable domain CDRs comprising RASENIYSNLA (SEQ ID NO: 4), ASTNLAD (SEQ ID NO: 26), and QHFWDTPYT (SEQ ID NO: 27).

7. The antibody or antigen-binding fragment thereof of claim 1, comprising heavy chain variable domain CDRs comprising GYFMN (SEQ ID NO: 30), RINPYNGDIFYNQKFKG (SEQ ID NO: 31), and GITVAAGSFDV (SEQ ID NO: 32); and
light chain variable domain CDRs comprising RASESVDNYGNTFMH (SEQ ID NO: 33), RASNLES (SEQ ID NO: 34), and QQSSDHPLT (SEQ ID NO: 35).

8. The antibody or antigen-binding fragment thereof of claim 1, wherein the light chain variable domain comprises an amino acid sequence that is at least 80% identical to the amino acid sequence:
(SEQ ID NO: 8)
DIQMTQSPASLSISVGETVTITCRASENIYSNLAWYQQKQGKSPQVLVY
GATNLADGVPSRFSGSGSGTQYSLKIKSLQSEDFGSYYCQHFWDSPFTF
GSGTKLEIK,
(SEQ ID NO: 16)
VIVLTQSPASLAVSLGQRAAISCRASESVDSYGYSFLHWYQQKPGQPPK
LLIYLASNLESGIPARFSGSGSGTDFTLTINPVEADDVATYYCQQSNED
LMYTFGGGTKLEIK,
(SEQ ID NO: 22)
DIQMTQSPASLSVSVGETVTITCRASENIYSNLAWYQQKQGKSPQLLVY
AATSLADGVPSRFRGSGSGTQYSLKISSLQSEDFGNYYCQNFWDTPYTF
GGGTKLEIK,
(SEQ ID NO: 29)
DIQMTQSPASLSVSVGETVTITCRASENIYSNLAWYQQKQGKSPQLLVY
ASTNLADGAPATFSGSGSGTQYSLKINSLQSVDFGSYYCQHFWDTPYTF
GGGTKLEIK,
(SEQ ID NO: 37)
DIVLTQSPASLAVSLGQRATISCRASESVDNYGNTFMHWYQQKPGQPPK
LLIYRASNLESGIPARFSGSGSKTDFTLTINPVEADDVATYYCQQSSDH
PLTFGAGTKLELS,
(SEQ ID NO: 45)
QIVLTQSPALMSASPGEKVTMTCSASSSVSFMYWYQQKPRSSPKPWIYL
TSNLASGVPARFSGSGSGTSYSLTISSMEAEDAATYYCQQWSSNPPTFG
GGTKLEIK,
(SEQ ID NO: 51)
DIQMTQSPASLSVSVGETVTITCRASENIYSNLAWYQQKQGKSPQLLVY
GATNLADGVPSRFGGSGSGTQYSLKINSLQPEDFGSYYCQHFWGTPYTF
GGGTKLEIT,
(SEQ ID NO: 59)
QAVVTQESALTTSPGETVTLTCRSSTGTVTTSNYANWVQEKPDHLFTGL
IGGTNNRAPGVPARFSGSLIGDQAALTITGAQTEDEAIYFCALWYSNHW
VFGGGTKLTVL,
(SEQ ID NO: 67)
SIVMTQTPKFLLVSAGDRVTITCKASQSVSDDVAWYQQKPGQSPKLLIY
YASNRYTGVPDRFTGSGYGTDFTFTISTVQAEDLAVYFCQQDYGSPTFG
GGTKLEIK,
or
(SEQ ID NO: 74)
DIQMTQSPASLSASVGETVTITCRASENIYSNLAWYQQKQGKSPQLLVS
AATNLADGVPSRFSGSGSGTQFSLKINSLQPEDFGSYYCQHFFGAPWTF
GGGTKLEIK,
or
(SEQ ID NO: 125)
DIQMTQSPSSLSASVGDRVTITCRASENIYSNLAWYQQKPGKAPKVLVY
GATNLADGVPSRFSGSGSGTEYTLTISSLQPEDFATYYCQHFWDSPFTF
GGGTKVEIKRTVAAPSVFIFPPSDEQLKSGTASVVCLLNNFYPREAKVQ
WKVDNALQSGNSQESVTEQDSKDSTYSLSSTLTLSKADYEKHKVYACEV
THQGLSSPVTKSFNRGEC;
and
wherein the heavy chain variable domain comprises an amino acid sequence that is at least 80% identical to the amino acid sequence:
(SEQ ID NO: 7)
EVQLQQSGPELVKPGASVKISCKASGYTFTDYYMNWVKQSHGKSLEWIG
DINPNNGGTSYNQKFKGKATLTVDKSSSTAYMELRSLTSEDSAVYYCAR
GGAELTGTYWYFDVWGTGTTVTVSS,
(SEQ ID NO: 15)
DVQLQQSGPELVKPGNSVKISCKAAGYSFTGYSVNWVKERHGKSLEWIG
RINPYNGDIFNNQKFKGKATLTVDKSSSTAHMELRSLTSEDSAVYYCAR
GTTVGGAWFAYWGQGTLVTVSA,
(SEQ ID NO: 21)
QVQLKQSGAELVRPGASVKLSCRASGYTFTDYYMNWVKQRPGQGLEWIA
RIYPGVYRTHYNEKFKDKATLTAEKSSSTAYMELSSLTSEDSAVYFCAR
SGYYGGTYEEDAMDYWGQGTSVTVSS,
(SEQ ID NO: 28)
EVQLQQSGPELVIPGASVKISCKASGYTFTDSYMNWVKQSHGKSLEWIA
YINPDNGVTRYNQKFKGKATLTVHKSSSTAYMELRSLTSEDSAVYYCAR
EGTITTDLSWFAYWGQGTLVTVSA,
(SEQ ID NO: 36)
EVHLQQSGPELVKPGASVKISCKASGYSFIGYFMNWMKQSHGKSLEWIG
RINPYNGDIFYNQKFKGKATLTVDKSSTTAHMDLLSLTSEDFAVYYCAR
GITVAAGSFDVWGTGTTVTVSS,
(SEQ ID NO: 44)
QVQLQQSGAELVKPGASVKISCKASGYAFSSYWMNWVKQRPGKGLEWIG
QIYPGHGDTNYNGKFKGKATLTADKSSSTAYMQLSSLTSEDSAVYFCAK
EGSELGRLFAYWGQGTLVTVSA,
(SEQ ID NO: 50)
QVQLQQSGTELVKPGASVKLSCKASGYIFTDYTIHWVKQRSGQGLEWIG
WFYPGTVSIKYNEKFKDKATLTADRSSSIVYMELSRLTSEDSGVYFCAR
HEHPHYYGDSYDAMGYWGQGTSVTVSS,
(SEQ ID NO: 58)
QVQLQQSGPELVKPGASVKISCKASGYIFTDNYLQWVKQRPGQGLEWIG
WIFPGSGNTYYSDNFTGKATLTVDKSSITAYMLLSSLTSEDSAVYFCSR
STVVYFDVWGTGTTVTVSS,
(SEQ ID NO: 66)
EVQLVESGGGLVKPGGSLKLSCAASGFTFSDYGMHWVRQAPEKRLEWVA
YISSDSSTIYYADTVKGRFTISRDNAKNTLFLEMTSLRSEDTAMYYCAR
RAAQGYVMDYWGQGTSVTVSS,
or
(SEQ ID NO: 73)
QIQLVQSGPELKKPGETVKISCKASGYTFTTYGMSWVKQAPGKGLKWMA
WINTYSGEPTYADEFKGRFAFSLETSVSTAYLQINNLKNEDTATYFCAR
RGYDGYYYTMDYWGQGTSVTVSS;
or
(SEQ ID NO: 124)
QVQLVQSGAEVKKPGASVKVSCKASGYTFTDYYMNWVRQAPGQRLEWIG
DINPNNGGTSYNQKFKGRATITVDTSASTAYMELSSLRSEDTAVYYCAR
GGAELTGTYWYFDVWGQGTTVTVSSASTKGPSVFPLAPSSKSTSGGTAA
LGCLVKDYFPEPVTVSWNSGALTSGVHTFPAVLQSSGLYSLSSVVTVPS
SSLGTQTYICNVNHKPSNTKVDKKVEPKSCDKTHTCPPCPAPEAAGGPS
VFLFPPKPKDTLMISRTPEVTCVVVDVSHEDPEVKENWYVDGVEVHNAK
TKPREEQYNSTYRVVSVLTVLHQDWLNGKEYKCKVSNKALPAPIEKTIS
KAKGQPREPQVYTLPPSRDELTKNQVSLTCLVKGFYPSDIAVEWESNGQ
PENNYKTTPPVLDSDGSFFLYSKLTVDKSRWQQGNVFSCSVMHEALHNH
YTQKSLSLSPGK.

9. (canceled)

10. The antibody or antigen-binding fragment thereof of claim 1, wherein the light chain variable domain comprises an amino acid sequence that is at least 80% identical to the amino acid sequence:
(SEQ ID NO: 8)
DIQMTQSPASLSISVGETVTITCRASENIYSNLAWYQQKQGKSPQVLVY
GATNLADGVPSRFSGSGSGTQYSLKIKSLQSEDFGSYYCQHFWDSPFTF
GSGTKLEIK,
(SEQ ID NO: 16)
VIVLTQSPASLAVSLGQRAAISCRASESVDSYGYSFLHWYQQKPGQPPK
LLIYLASNLESGIPARFSGSGSGTDFTLTINPVEADDVATYYCQQSNED
LMYTFGGGTKLEIK,
(SEQ ID NO: 22)
DIQMTQSPASLSVSVGETVTITCRASENIYSNLAWYQQKQGKSPQLLVY
AATSLADGVPSRFRGSGSGTQYSLKISSLQSEDFGNYYCQNFWDTPYTF
GGGTKLEIK,
(SEQ ID NO: 29)
DIQMTQSPASLSVSVGETVTITCRASENIYSNLAWYQQKQGKSPQLLVY
ASTNLADGAPATFSGSGSGTQYSLKINSLQSVDFGSYYCQHFWDTPYTF
GGGTKLEIK,
or
(SEQ ID NO: 37)
DIVLTQSPASLAVSLGQRATISCRASESVDNYGNTFMHWYQQKPGQPPK
LLIYRASNLESGIPARFSGSGSKTDFTLTINPVEADDVATYYCQQSSDH
PLTFGAGTKLELS.

11. The antibody or antigen-binding fragment thereof of claim 1, wherein the heavy chain variable domain comprises an amino acid sequence that is at least 80% identical to the amino acid sequence:
(SEQ ID NO: 7)
EVQLQQSGPELVKPGASVKISCKASGYTFTDYYMNWVKQSHGKSLEWIG
DINPNNGGTSYNQKFKGKATLTVDKSSSTAYMELRSLTSEDSAVYYCAR
GGAELTGTYWYFDVWGTGTTVTVSS,
(SEQ ID NO: 15)
DVQLQQSGPELVKPGNSVKISCKAAGYSFTGYSVNWVKERHGKSLEWIG
RINPYNGDIFNNQKFKGKATLTVDKSSSTAHMELRSLTSEDSAVYYCAR
GTTVGGAWFAYWGQGTLVTVSA,
(SEQ ID NO: 21)
QVQLKQSGAELVRPGASVKLSCRASGYTFTDYYMNWVKQRPGQGLEWIA
RIYPGVYRTHYNEKFKDKATLTAEKSSSTAYMELSSLTSEDSAVYFCAR
SGYYGGTYEEDAMDYWGQGTSVTVSS,
(SEQ ID NO: 28)
EVQLQQSGPELVIPGASVKISCKASGYTFTDSYMNWVKQSHGKSLEWIA
YINPDNGVTRYNQKFKGKATLTVHKSSSTAYMELRSLTSEDSAVYYCAR
EGTITTDLSWFAYWGQGTLVTVSA,
or
(SEQ ID NO: 36)
EVHLQQSGPELVKPGASVKISCKASGYSFIGYFMNWMKQSHGKSLEWIG
RINPYNGDIFYNQKFKGKATLTVDKSSTTAHMDLLSLTSEDFAVYYCAR
GITVAAGSFDVWGTGTTVTVSS.

12. The antibody or antigen-binding fragment thereof of claim 1, wherein the antibody or antigen-binding fragment thereof antagonizes the interaction between ILT4 and HLA-G or HLA-A.

13. The antibody or antigen-binding fragment thereof of claim 1, wherein the antibody or antigen-binding fragment thereof selectively binds to ILT4 relative to other leukocyte immunoglobulin-like receptor family members.

14. The antibody or antigen-binding fragment thereof of claim 1, wherein the antibody or antigen-binding fragment thereof comprises: a) a heavy chain constant region selected from the group consisting of IgG1, IgG2, or IgG4; b) a light chain constant region selected from the group consisting of kappa and lambda; or c) both a) and b).

15. (canceled)

16. The antibody or antigen-binding fragment thereof of claim 1, comprising a variant Fc region of IgG1, IgG2, or IgG4,
wherein the variant Fc region of IgG1 comprises:
a) an amino acid substitution at position Leu234 with alanine;
b) an amino acid substitution at position Leu235 with alanine;
c) an amino acid substitution at position Pro329 with glycine or arginine;
d) an amino acid substitution at position Asn297 with alanine;
e) an amino acid substitution at position Asn297 with Glutamine;
f) an amino acid substitution at position Asn297 with Glycine;
g) any combination of (a) to (f);
wherein the variant Fc region of IgG2 comprises:
h) an amino acid substitution at position Ser228 with proline;
i) an amino acid substitution at position Pro329 with glycine or arginine; or
j) both (h) and (i); and
wherein the variant Fc region of IgG4 comprises:
k) an amino acid substitution at position Ser228 with proline;
l) an amino acid substitution at position Leu235 with alanine or glutamate;
m) an amino acid substitution at position Pro329 with glycine or arginine; or
n) any combination of (k) to (m),
wherein the residues are numbered according to the EU index of Kabat.

17. The antibody or antigen-binding fragment thereof of claim 1, wherein the antibody or antigen-binding fragment thereof a) specifically binds to human ILT4 with at least 100× greater affinity than the antibody or antigen-binding fragment thereof binds to each of human ILT3 and human ILT2; b) binds to ILT4 with an equilibrium disassociation constant (K_{D}) of less than or equal to 1 nM, less than or equal to 0.9 nM, less than or equal to 0.8 nM, less than or equal to 0.7 nM, less than or equal to 0.6 nM, less than or equal to 0.5 nM, less than or equal to 0.4 nM, less than or equal to 0.3 nM, less than or equal to 0.2 nM, less than or equal to 0.1 nM, less than or equal to 0.09 nM, or less than or equal to 0.08 nM; or c) both a) and b).

18. (canceled)

19. The antibody or antigen-binding fragment thereof of claim 1, wherein the antibody or antigen-binding fragment thereof: a) binds to ILT4 with an equilibrium disassociation constant (K_{D}) of about 0.5 to about 10 pM, about 1 to about 9 pM, about 2 to about 8 pM, about 3 to about 7 pM, or about 4 to about 6 pM; b) has an IC₅₀ value of blocking HLA-G binding to ILT4 of about 0.1 to about 0.25 μg/mL; c) has an IC₅₀ value of blocking HLA-A binding to ILT4 of about 0.1 to about 0.2 μg/mL; d) has an EC₅₀ value of TNF-alpha production of about 7 ng/ml to about 25 ng/ml or about 12 ng/ml to about 20 ng/ml; e) has an EC₅₀ value of IL-6 production of about 5 ng/ml to about 25 ng/ml, about 6 ng/mL to about 22 ng/ml, or about 8 ng/ml to about 21 ng/mL; or f) any combination of (a)-(e).

20. (canceled)

21. (canceled)

22. (canceled)

23. (canceled)

24. An immunoconjugate comprising the antibody or antigen-binding fragment thereof of claim 1 coupled to a chemotherapeutic agent, a cytotoxin, a detectable moiety, a diagnostic agent, or a combination thereof.

25. A method of treating a subject having an autoimmune, neoplastic, or inflammatory disorder comprising administering the antibody or antigen-binding fragment thereof of claim 1 to the subject.

26. The method of claim 25, wherein the autoimmune disease is Guillain-Barre syndrome, Chronic inflammatory demyelinating polyneuropathy, ankylosing spondylitis, psoriatic arthritis, enteropathic arthritis, reactive arthritis, undifferentiated spondyloarthropathy, juvenile spondyloarthropathy, Behcet's disease, enthesitis, ulcerative colitis, Crohn's disease, irritable bowel syndrome, inflammatory bowel disease, fibromyalgia, chronic fatigue syndrome, pain conditions associated with systemic inflammatory disease, systemic lupus erythematosus, Sjogren's syndrome, rheumatoid arthritis, juvenile rheumatoid arthritis, juvenile onset diabetes mellitus (also known as Type I diabetes mellitus), Wegener's granulomatosis, polymyositis, dermatomyositis, inclusion body myositis, multiple endocrine failure, Schmidt's syndrome, autoimmune uveitis, Addison's disease, Grave's Disease, Hashimoto's thyroiditis, autoimmune thyroid disease, pernicious anemia, gastric atrophy, chronic hepatitis, lupoid hepatitis, atherosclerosis, multiple sclerosis, amyotrophic lateral sclerosis, hypoparathyroidism, Dressler's syndrome, myasthenia gravis, Eaton-Lambert syndrome, autoimmune thrombocytopenia, idiopathic thrombocytopenia purpura, hemolytic anemia, pemphigus vulgaris, pemphigus, dermatitis herpetiformis, alopecia, scleroderma, progressive systemic sclerosis, CREST syndrome (calcinosis, Raynaud's phenomenon, esophageal dysmotility, sclerodactyly, and telangtasia), adult onset diabetes mellitus (also known as Type II diabetes mellitus), mixed connective tissue disease, polyarteritis nodosa, systemic necrotizing vasculitis, glomerulonephritis, atopic dermatitis, atopic rhinitis, Goodpasture's syndrome, Chagas' disease, sarcoidosis, rheumatic fever, asthma, anti-phospholipidsyndrome, erythema multiforme, Cushing's syndrome, autoimmune chronic active hepatitis, allergic disease, allergic encephalomyelitis, transfusion reaction, leprosy, malaria, leshmaniasis, trypanosomiasis, Takayasu's arteritis, polymyalgia rheumatica, temporal arteritis, shistosomiasis, giant cell arteritis, eczema, lymphomatoid granulomatosis, Kawasaki's disease, endophthalmitis, psoriasis, erythroblastosis fetalis, eosinophilic faciitis, Shulman's syndrome, Felty's syndrome, Fuch's cyclitis, IgA nephropathy, Henoch-Schonlein purpura, graft versus host disease, transplantation rejection, tularemia, periodic fever syndromes, pyogenic arthritis, Familial Mediterranean Fever, TNF-receptor associated periodic syndrome (TRAPS), Muckle-Wells syndrome, or hyper-IgD syndrome.

27. A method of delivering the antibody or antigen-binding fragment thereof of claim 1 to a subject comprising administering the antibody or antigen-binding fragment thereof to the subject via a route of administration selected from parenteral, subcutaneous, intramuscular, intravenous, intrarticular, intrabronchial, intraabdominal, intracapsular, intracartilaginous, intracavitary, intracelial, intracerebellar, intracerebroventricular, intracolic, intracervical, intragastric, intrahepatic, intramyocardial, intraosteal, intrapelvic, intrapericardiac, intraperitoneal, intrapleural, intraprostatic, intrapulmonary, intrarectal, intrarenal, intraretinal, intraspinal, intrathecal, intra-Ommaya, intravitreous, intraocular, intrasynovial, intrathoracic, intrauterine, intravesical, bolus, vaginal, rectal, buccal, sublingual, intranasal, or transdermal.

28. An expression system comprising at least one expression vector,
wherein the expression system comprises one or more nucleic acid sequences encoding a protein comprising the antibody or antigen-binding fragment thereof of claim 1.

29. A host cell comprising the expression system of claim 28.

30. The host cell of claim 29, wherein the host cell is a mammalian cell.

31. The host cell of claim 29, wherein the host cell is a Chinese Hamster Ovary cell.

32. A method of manufacturing an antibody or antigen-binding fragment thereof comprising:
culturing the host cell of claim 29 under conditions for expressing the antibody or antigen-binding fragment thereof, and recovering the antibody or antigen-binding fragment thereof.

33. The method of claim 32, further comprising purifying the recovered antibody or antigen-binding fragment thereof.

34. A pharmaceutical composition comprising the antibody or antigen-binding fragment thereof of claim 1, and a pharmaceutically acceptable carrier.

35. The pharmaceutical composition of claim 34, further comprising an additional therapeutic agent.

36. The pharmaceutical composition of claim 35, wherein the additional therapeutic agent is an antagonist of PD-1/PD-L1, TIGIT, or CTLA4.

37. The pharmaceutical composition of claim 36, wherein the antagonist of PD-L/PD-1 is selected from the group consisting of nivolumab, pembrolizumab, atezolizumab, durvalumab, lambrolizumab, avelumab, and toripalimab.

38. The pharmaceutical composition of claim 37, wherein the antagonist of PD-L/PD-1 is toripalimab.

39. A method of treating a subject having a cancer, an autoimmune disease, or an inflammatory disease, comprising administering the pharmaceutical composition of claim 34 to the subject, optionally further comprising administering an additional therapeutic agent that is an antagonist of PD-1/PD-L1, TIGIT, or CTLA4.

40. The method of claim 39, wherein the cancer is a solid cancer or a liquid cancer.

41. The method of claim 39, wherein the cancer is a carcinoma or a sarcoma.

42. The method of claim 39, wherein the cancer is squamous cell carcinoma, small-cell lung cancer, non-small cell lung cancer, squamous non-small cell lung cancer (NSCLC), nonsquamous NSCLC, glioma, gastrointestinal cancer, renal cancer (e.g., clear cell carcinoma), ovarian cancer, liver cancer, colorectal cancer, endometrial cancer, kidney cancer (e.g., renal cell carcinoma (RCC)), prostate cancer (e.g., hormone refractory prostate adenocarcinoma), thyroid cancer, neuroblastoma, pancreatic cancer, glioblastoma (glioblastoma multiforme), cervical cancer, stomach cancer, bladder cancer, hepatoma, breast cancer, colon carcinoma, head and neck cancer (or carcinoma), gastric cancer, germ cell tumor, pediatric sarcoma, sinonasal natural killer, melanoma (e.g., metastatic malignant melanoma, such as cutaneous or intraocular malignant melanoma), bone cancer, skin cancer, uterine cancer, cancer of the anal region, testicular cancer, carcinoma of the fallopian tubes, carcinoma of the endometrium, carcinoma of the cervix, carcinoma of the vagina, carcinoma of the vulva, cancer of the esophagus, cancer of the small intestine, cancer of the endocrine system, cancer of the parathyroid gland, cancer of the adrenal gland, sarcoma of soft tissue, cancer of the urethra, cancer of the penis, solid tumors of childhood, cancer of the ureter, carcinoma of the renal pelvis, neoplasm of the central nervous system (CNS), primary CNS lymphoma, tumor angiogenesis, spinal axis tumor, brain cancer, brain stem glioma, pituitary adenoma, Ewing's sarcoma, Kaposi's sarcoma, epidermoid cancer, squamous cell cancer, T-cell lymphoma, mesothelioma, meningioma, chondrosarcoma, pituitary adenoma, vestibular schwannoma, primitive neuroectodermal tumor, medulloblastoma, astrocytoma, anaplastic astrocytoma, oligodendroglioma, ependymoma, choroid plexus papilloma, polycythemia vera, thrombocythemia, idiopathic myelfibrosis, soft tissue sarcoma, thyroid cancer, endometrial cancer, and carcinoid cancer, a leukemia, a lymphoma, a myeloma, Wilm's cancer, or a combination or metastasis thereof.

43. The method of claim 39, wherein the cancer is acute leukemia (ALL), acute myelogenous leukemia (AML), chronic lymphocytic leukemia (CLL), and chronic myelogenous leukemia (CML), undifferentiated AML (MO), myeloblastic leukemia (M1), myeloblastic leukemia (M2; with cell maturation), promyelocytic leukemia (M3 or M3 variant [M3V]), myelomonocytic leukemia (M4 or M4 variant with eosinophilia [M4E]), monocytic leukemia (M5), erythroleukemia (M6), megakaryoblastic leukemia (M7), isolated granulocytic sarcoma, and chloroma; lymphomas, such as Hodgkin's lymphoma (HL), non-Hodgkin's lymphoma (NHL), a B-cell lymphoma, a T-cell lymphoma, lymphoplasmacytoid lymphoma, monocytoid B-cell lymphoma, mucosa-associated lymphoid tissue (MALT) lymphoma, anaplastic (e.g., Ki 1+) large-cell lymphoma, adult T-cell lymphoma/leukemia, mantle cell lymphoma, angio immunoblastic T-cell lymphoma, angiocentric lymphoma, intestinal T-cell lymphoma, primary mediastinal B-cell lymphoma, precursor T-lymphoblastic lymphoma, T-lymphoblastic; and lymphoma/leukaemia (T-Lbly/T-ALL), peripheral T-cell lymphoma, lymphoblastic lymphoma, post-transplantation lymphoproliferative disorder, true histiocytic lymphoma, primary central nervous system lymphoma, primary effusion lymphoma, B cell lymphoma, lymphoblastic lymphoma (LBL), hematopoietic tumors of lymphoid lineage, acute lymphoblastic leukemia, diffuse large B-cell lymphoma, Burkitt's lymphoma, follicular lymphoma, diffuse histiocytic lymphoma (DHL), immunoblastic large cell lymphoma, precursor B-lymphoblastic lymphoma, cutaneous T-cell lymphoma (CTLC) (also called mycosis fungoides or Sezary syndrome), and lymphoplasmacytoid lymphoma (LPL) with Waldenstrom's macroglobulinemia; myelomas, such as IgG myeloma, light chain myeloma, nonsecretory myeloma, smoldering myeloma (also called indolent myeloma), solitary plasmocytoma, and multiple myelomas, chronic lymphocytic leukemia (CLL), hairy cell lymphoma; hematopoietic tumors of myeloid lineage, tumors of mesenchymal origin, including fibrosarcoma and rhabdomyoscarcoma; seminoma, teratocarcinoma, tumors of the central and peripheral nervous, including astrocytoma, schwannomas; tumors of mesenchymal origin, including fibrosarcoma, rhabdomyoscaroma, and osteosarcoma; and other tumors, including melanoma, xeroderma pigmentosum, keratoacanthoma, seminoma, thyroid follicular cancer and teratocarcinoma, hematopoietic tumors of lymphoid lineage, for example T-cell and B-cell tumors, including but not limited to T-cell disorders such as T-prolymphocytic leukemia (T-PLL), including of the small cell and cerebriform cell type; large granular lymphocyte leukemia (LGL) of the T-cell type; a/d T-NHL hepatosplenic lymphoma; peripheral/post-thymic T cell lymphoma (pleomorphic and immunoblastic subtypes); angiocentric (nasal) T-cell lymphoma; cancer of the head or neck, renal cancer, rectal cancer, cancer of the thyroid gland; acute myeloid lymphoma, or a combination or metastasis thereof.

44. The method of claim 39, wherein the cancer is a lung cancer, liver cancer, ovarian cancer, pancreatic cancer, cervical cancer, skin cancer, bladder cancer, colon cancer, breast cancer, glioma, glioblastoma, renal carcinoma, stomach cancer, gastric cancer, esophageal cancer, oral squamous cell cancer, or head/neck cancer.

45. The method of claim 39, wherein the cancer is a NSCLC.

46. A method of inducing apoptosis of cancer cells or cancer stem cells comprising contacting the cancer cells or cancer stem cells with an antibody or antigen-binding fragment thereof of claim 1.

47. An antibody or antigen-binding fragment thereof that binds ILT4, comprising a light chain variable domain comprising an amino acid sequence that is at least 90% identical to:
(SEQ ID NO: 8)
DIQMTQSPASLSISVGETVTITCRASENIYSNLAWYQQKQGKSPQVLVY
GATNLADGVPSRFSGSGSGTQYSLKIKSLQSEDFGSYYCQHFWDSPFTF
GSGTKLEIK,
(SEQ ID NO: 16)
VIVLTQSPASLAVSLGQRAAISCRASESVDSYGYSFLHWYQQKPGQPPK
LLIYLASNLESGIPARFSGSGSGTDFTLTINPVEADDVATYYCQQSNED
LMYTFGGGTKLEIK,
(SEQ ID NO: 22)
DIQMTQSPASLSVSVGETVTITCRASENIYSNLAWYQQKQGKSPQLLVY
AATSLADGVPSRFRGSGSGTQYSLKISSLQSEDFGNYYCQNFWDTPYTF
GGGTKLEIK,
(SEQ ID NO: 29)
DIQMTQSPASLSVSVGETVTITCRASENIYSNLAWYQQKQGKSPQLLVY
ASTNLADGAPATFSGSGSGTQYSLKINSLQSVDFGSYYCQHFWDTPYTF
GGGTKLEIK,
(SEQ ID NO: 37)
DIVLTQSPASLAVSLGQRATISCRASESVDNYGNTFMHWYQQKPGQPPK
LLIYRASNLESGIPARFSGSGSKTDFTLTINPVEADDVATYYCQQSSDH
PLTFGAGTKLELS,
(SEQ ID NO: 45)
QIVLTQSPALMSASPGEKVTMTCSASSSVSFMYWYQQKPRSSPKPWIYL
TSNLASGVPARFSGSGSGTSYSLTISSMEAEDAATYYCQQWSSNPPTFG
GGTKLEIK,
(SEQ ID NO: 51)
DIQMTQSPASLSVSVGETVTITCRASENIYSNLAWYQQKQGKSPQLLVY
GATNLADGVPSRFGGSGSGTQYSLKINSLQPEDFGSYYCQHFWGTPYTF
GGGTKLEIT,
(SEQ ID NO: 59)
QAVVTQESALTTSPGETVTLTCRSSTGTVTTSNYANWVQEKPDHLFTGL
IGGTNNRAPGVPARFSGSLIGDQAALTITGAQTEDEAIYFCALWYSNHW
VFGGGTKLTVL,
(SEQ ID NO: 67)
SIVMTQTPKFLLVSAGDRVTITCKASQSVSDDVAWYQQKPGQSPKLLIY
YASNRYTGVPDRFTGSGYGTDFTFTISTVQAEDLAVYFCQQDYGSPTFG
GGTKLEIK,
or
(SEQ ID NO: 74)
DIQMTQSPASLSASVGETVTITCRASENIYSNLAWYQQKQGKSPQLLVS
AATNLADGVPSRFSGSGSGTQFSLKINSLQPEDFGSYYCQHFFGAPWTF
GGGTKLEIK;
and
a heavy chain variable domain comprising an amino
acid sequence that is at least 90% identical to:
(SEQ ID NO: 7)
EVQLQQSGPELVKPGASVKISCKASGYTFTDYYMNWVKQSHGKSLEWIG
DINPNNGGTSYNQKFKGKATLTVDKSSSTAYMELRSLTSEDSAVYYCAR
GGAELTGTYWYFDVWGTGTTVTVSS,
(SEQ ID NO: 15)
DVQLQQSGPELVKPGNSVKISCKAAGYSFTGYSVNWVKERHGKSLEWIG
RINPYNGDIFNNQKFKGKATLTVDKSSSTAHMELRSLTSEDSAVYYCAR
GTTVGGAWFAYWGQGTLVTVSA,
(SEQ ID NO: 21)
QVQLKQSGAELVRPGASVKLSCRASGYTFTDYYMNWVKQRPGQGLEWIA
RIYPGVYRTHYNEKFKDKATLTAEKSSSTAYMELSSLTSEDSAVYFCAR
SGYYGGTYEEDAMDYWGQGTSVTVSS,
(SEQ ID NO: 28)
EVQLQQSGPELVIPGASVKISCKASGYTFTDSYMNWVKQSHGKSLEWIA
YINPDNGVTRYNQKFKGKATLTVHKSSSTAYMELRSLTSEDSAVYYCAR
EGTITTDLSWFAYWGQGTLVTVSA,
(SEQ ID NO: 36)
EVHLQQSGPELVKPGASVKISCKASGYSFIGYFMNWMKQSHGKSLEWIG
RINPYNGDIFYNQKFKGKATLTVDKSSTTAHMDLLSLTSEDFAVYYCAR
GITVAAGSFDVWGTGTTVTVSS,
(SEQ ID NO: 44)
QVQLQQSGAELVKPGASVKISCKASGYAFSSYWMNWVKQRPGKGLEWIG
QIYPGHGDTNYNGKFKGKATLTADKSSSTAYMQLSSLTSEDSAVYFCAK
EGSELGRLFAYWGQGTLVTVSA,
(SEQ ID NO: 50)
QVQLQQSGTELVKPGASVKLSCKASGYIFTDYTIHWVKQRSGQGLEWIG
WFYPGTVSIKYNEKFKDKATLTADRSSSIVYMELSRLTSEDSGVYFCAR
HEHPHYYGDSYDAMGYWGQGTSVTVSS,
(SEQ ID NO: 58)
QVQLQQSGPELVKPGASVKISCKASGYIFTDNYLQWVKQRPGQGLEWIG
WIFPGSGNTYYSDNFTGKATLTVDKSSITAYMLLSSLTSEDSAVYFCSR
STVVYFDVWGTGTTVTVSS,
(SEQ ID NO: 66)
EVQLVESGGGLVKPGGSLKLSCAASGFTFSDYGMHWVRQAPEKRLEWVA
YISSDSSTIYYADTVKGRFTISRDNAKNTLFLEMTSLRSEDTAMYYCAR
RAAQGYVMDYWGQGTSVTVSS,
or
(SEQ ID NO: 73)
QIQLVQSGPELKKPGETVKISCKASGYTFTTYGMSWVKQAPGKGLKWMA
WINTYSGEPTYADEFKGRFAFSLETSVSTAYLQINNLKNEDTATYFCAR
RGYDGYYYTMDYWGQGTSVTVSS.

48. The antibody or antigen-binding fragment thereof of claim 47, comprising a light chain variable domain comprising an amino acid sequence that is at least 91%, at least 92%, at least 93%, at least 94%, at least 95%, at least 96%, at least 97%, at least 98%, or at least 99% identical to the amino acid sequence set forth in SEQ ID NOs: 8, 16, 22, 29, 37, 45, 51, 59, 67, or 74; and a heavy chain variable domain comprising an amino acid sequence that is at least 91%, at least 92%, at least 93%, at least 94%, at least 95%, at least 96%, at least 97%, at least 98%, or at least 99% identical to the amino acid sequence set forth in SEQ ID NOS: 7, 15, 21, 28, 36, 44, 50, 58, 66, or 73.

49. The antibody or antigen-binding fragment thereof of claim 47, comprising a light chain variable domain comprising an amino acid sequence that is at least 91%, at least 92%, at least 93%, at least 94%, at least 95%, at least 96%, at least 97%, at least 98%, or at least 99% identical to the amino acid sequence set forth in SEQ ID NOs: 8, 16, 22, 29, or 37; and a heavy chain variable domain comprising an amino acid sequence that is at least 91%, at least 92%, at least 93%, at least 94%, at least 95%, at least 96%, at least 97%, at least 98%, or at least 99% identical to the amino acid sequence set forth in SEQ ID NOs: 7, 15, 21, 28, or 36.

50. The antibody or antigen-binding fragment thereof of claim 47, comprising a light chain variable domain comprising an amino acid sequence set forth in SEQ ID NOs: 8, 16, 22, 29, or 37; and a heavy chain variable domain comprising an amino acid sequence set forth in SEQ ID NOs: 7, 15, 21, 28, or 36.

51. The antibody or antigen-binding fragment thereof of claim 47, comprising:
a CDR-H1 comprising an amino acid sequence of X₁X₂X₃X₄N, wherein X₁ is D or G, X₂ is Y or S, X₃ is Y, S, or F, and X₄ is M or V,
a CDR-H2 comprising an amino acid sequence of X₁IX₂PX₃X₄X₅X₆X₇X₈X₉NX₁₀KFKX₁₁ (SEQ ID No: 76), wherein X₁ is R, D, or Y; X₂ is N or Y; X₃ is G, N, D, or Y; X₄ is V or N; X₅ is G or Y; X₆ is R, G, V, or D; X₇ is I or T; X₈ is H, S, R, of F; X₉ is Y or N; X₁₀ is E or Q; X₁₁ is D or G,
and a CDR-H3 comprising an amino acid sequence of X₁X₂X₃X₄X₅X₆X₇X₈X₉X₁₀X₁₁X₁₂X₁₃X₁₄X₁₅, wherein X₁ is G, S, or E; X₂ is G, T, or I; X₃ is A, T, or Y; X₄ E, V, Y, or I; X₅ is L, G, T, or A; X₆ is T, G, or A; X₇ is G, A, T, or D; X₈ is T, W, Y, L, or S; X₉ is Y, F, E, or S; X₁₀ is W, A, E, or D; X₁₁ is Y, D, F, or V; X₁₂ is F, A, or absent; X₁₃ is D, M, Y, or absent; X₁₄ is V, D, or absent; and X₁₅ is Y or absent; and
a light chain variable domain comprising:
a CDR-L1 comprising an amino acid sequence of RASEX₁X₂X₃X₄X₅X₆X₇X₈X₉ X₁₀ (SEQ ID NO: 78), wherein X₁ is N or S; X₂ I or V; X₃ is Y or D; X₄ is S or N; X₅ is N or Y; X₆ is L or G; X₇ or A, Y, or N; X₈ is S, T, or absent; X₉ is F or absent; X₁₀ is L, M, or absent,
a CDR-L2 comprising an amino acid sequence of X₁X₂X₃X₄LX5X₆, wherein X₁ is L, R, G, or A; X₂ is A or S; X₃ is S or T; X₄ is N or S; X₅ is E or A; and X₆ is S or D, and
a CDR-L3 comprising an amino acid sequence of X₁QX₂X₃X₄DX₅X₆X₇T, wherein X₁ is absent or Q; X₂ is S, H, N, or Q; X₃ is N, F, or S; X₄ E, W, or S; X₅ is L, S, T, or H; X₆ is M or P; and X₇ is Y, F, or L.

52. The antibody or antigen-binding fragment thereof of claim 47, wherein the antibody or antigen-binding fragment thereof antagonizes the interaction between ILT4 and HLA-G or HLA-A.

53. The antibody or antigen-binding fragment thereof of claim 47, wherein the antibody or antigen-binding fragment thereof selectively binds to ILT4 relative to other leukocyte immunoglobulin-like receptor family members.

54. The antibody or antigen-binding fragment thereof of claim 47, wherein the antibody comprises a heavy chain constant region selected from the group consisting of IgG1, IgG2, or IgG4.

55. The antibody or antigen-binding fragment thereof of claim 47, wherein the antibody comprises a light chain constant region selected from the group consisting of kappa and lambda.

56. The antibody or antigen-binding fragment thereof of claim 47, comprising a variant Fc region of IgG1, IgG2, or IgG4,
wherein the variant Fc region of IgG1 comprises:
a) an amino acid substitution at position Leu234 with alanine;
b) an amino acid substitution at position Leu235 with alanine;
c) an amino acid substitution at position Pro329 with glycine or arginine;
d) an amino acid substitution at position Asn297 with alanine;
e) an amino acid substitution at position Asn297 with Glutamine;
f) an amino acid substitution at position Asn297 with Glycine; or
g) any combination of (a) to (f);
wherein the variant Fc region of IgG2 comprises:
h) an amino acid substitution at position Ser228 with proline;
i) an amino acid substitution at position Pro329 with glycine or arginine; or
j) both (h) and (i); and
wherein the variant Fc region of IgG4 comprises:
k) an amino acid substitution at position Ser228 with proline;
l) an amino acid substitution at position Leu235 with alanine or glutamate;
m) an amino acid substitution at position Pro329 with glycine or arginine; or
n) any combination of (k) to (m),
wherein the residues are numbered according to the EU index of Kabat.

57. The antibody or antigen-binding fragment thereof of claim 47, wherein the antibody or antigen-binding fragment thereof specifically binds to human ILT4 with at least 100× greater affinity than the antibody or antigen-binding fragment thereof binds to each of human ILT3 and human ILT2.

58. The antibody or antigen-binding fragment thereof of claim 47, wherein the antibody or antigen-binding fragment thereof binds to ILT4 with an equilibrium disassociation constant (K_{D}) of less than or equal to 1 nM, less than or equal to 0.9 nM, less than or equal to 0.8 nM, less than or equal to 0.7 nM, less than or equal to 0.6 nM, less than or equal to 0.5 nM, less than or equal to 0.4 nM, less than or equal to 0.3 nM, less than or equal to 0.2 nM, less than or equal to 0.1 nM, less than or equal to 0.09 nM, or less than or equal to 0.08 nM.

59. (canceled)

60. A method of treating a subject having an autoimmune, neoplastic, or inflammatory disorder comprising administering the antibody or antigen-binding fragment thereof of claim 47 to the subject.

61. (canceled)

62. (canceled)

63. (canceled)

64. (canceled)

65. (canceled)

66. (canceled)

67. (canceled)

68. (canceled)

69. A pharmaceutical composition comprising the antibody or antigen-binding fragment thereof of claim 47, and a pharmaceutically acceptable carrier.

70. The pharmaceutical composition of claim 69, further comprising an additional therapeutic agent.

71. The pharmaceutical composition of claim 70, wherein the additional therapeutic agent is an antagonist of PD-1/PD-L1, TIGIT, or CTLA4.

72. The pharmaceutical composition of claim 71, wherein the antagonist of PD-L/PD-1 is selected from the group consisting of nivolumab, pembrolizumab, atezolizumab, durvalumab, lambrolizumab, avelumab, and toripalimab.

73. The pharmaceutical composition of claim 73, wherein the antagonist of PD-L/PD-1 is toripalimab.

74. A method of treating a subject having a cancer, comprising administering the pharmaceutical composition of claim 69 to the subject, optionally further comprising administering an additional therapeutic agent that is an antagonist of PD-1/PD-L1, TIGIT, or CTLA4.

75. (canceled)

76. (canceled)

77. (canceled)

78. (canceled)

79. (canceled)

80. The method of claim 74, wherein the cancer is a NSCLC.

81. (canceled)

82. An antibody or antigen-binding fragment thereof that binds ILT4, comprising heavy chain variable domain CDRs comprising DYYMN (SEQ ID NO: 1), IYPGVYRT (SEQ ID NO: 113), and SGYYGGTYEEDAMDY (SEQ ID NO: 18); and
light chain variable domain CDRs comprising ENIYSN (SEQ ID NO: 114), AAT, and QNFWDTPYT (SEQ ID NO: 20), or
comprising heavy chain variable domain CDRs comprising DYYMN (SEQ ID NO: 1), INPNNGGT (SEQ ID NO: 116), and GGAELTGTYWYFDV (SEQ ID NO: 3); and
light chain variable domain CDRs comprising ENIYS (SEQ ID NO: 117), GAT, and QHFWDSPFT (SEQ ID NO: 6), or
comprising heavy chain variable domain CDRs comprising GYSVN (SEQ ID NO: 9), INPYNGDI (SEQ ID NO: 119), and GTTVGGAWFAY (SEQ ID NO: 11); and
light chain variable domain CDRs comprising ESVDSYGYSF (SEQ ID NO: 120), LAS, and QQSNEDLMYT (SEQ ID NO: 14), or
comprising heavy chain variable domain CDRs comprising GYFMN (SEQ ID NO: 30), INPYNGDI (SEQ ID NO: 119), and GITVAAGSFDV (SEQ ID NO: 32); and
light chain variable domain CDRs comprising ESVDNYGNTF (SEQ ID NO: 122), RAS, and QQSSDHPLT (SEQ ID NO: 35).

83. (canceled)

84. (canceled)

85. (canceled)
